(19) United States
(12) Patent Application Publication
(10) Pub. No.: US 2022/0390421 A1
(43) Pub. Date: Dec. 8, 2022

(54) MATERIALS AND METHODS FOR MASS SPECTROMETRIC PROTEIN ANALYSIS

(71) Applicant: Janssen Biotech, Inc., Horsham, PA (US)

(72) Inventors: HARSHA GUNAWARDENA, Spring House, PA (US); HIRSH NANDA, Spring House, PA (US)

(21) Appl. No.: 17/767,263

(22) PCT Filed: Oct. 9, 2020
(86) PCT No.: PCT/US20/54949

§ 371 Date: Apr. 7, 2022

Related U.S. Application Data
(60) Provisional application No. 62/913,406, filed on Oct. 10, 2019.

(51) Int. Cl. G01N 30/72

(52) U.S. Cl. CPC G01N 30/72; G01N 2030/8831

(57) ABSTRACT

The present invention describes systems, processes, and methods of characterizing a protein or a polypeptide and/or identifying clipping sites on a protein or a polypeptide. In some embodiments, the said systems, processes, and methods comprise generation of reporter ions and subsequent tandem mass spectrometry that is triggered by the reporter ion.

### CROSS-REFERENCE TO RELATED APPLICATIONS

This application claims priority to and the benefit of U.S. Provisional Application No. 62/913,406, filed Oct. 10, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “206389_0027_00WO_SequenceListing_ST25.txt” and a creation date of Oct. 5, 2020 and having a size of 10 KB. The sequence listing submitted via EFS-Web is part of the specification and is herein incorporated by reference in its entirety.

### FIELD OF THE INVENTION

The invention relates to methods of identifying and characterizing polypeptides as well as clipping sites on a protein or a polypeptide. In one aspect, the invention relates to methods for identifying clipping sites of a protein or a polypeptide via generation of reporter ion and subsequent tandem mass spectrometry that is triggered by the reporter ion.

### SUMMARY

Recent advances in protein engineering technologies have enabled novel therapeutic modalities that are unique in structural and conformational diversity (AlDeghaither et al., J. Clin. Pharmacol., 2015, 55, Suppl 3:S4-20). These new drugs are also prone to liabilities including fast proteolytic degradation that may impair efficacy and safety profiles. Clipping of proteins is ubiquitous and can take place in culture or during process development and most often attributed to host cell-derived proteases that are site specific or have a broad range of substrate specificities (Dorai et al., Biotechnology and Bioengineering, 2009, 103:162-176; Dorai et al., Biotechnology Progress, 2011, 27:220-231). Clipping can also take place via non-enzymatic mechanisms where a considerable number of sites that are prone to clipping are influenced by factors such as the type of side chain, alteration of local flexibility due to secondary, tertiary, and quaternary structures, and pre-analytical variables (e.g., pH, temperature, metals, and radicals) often used in the assessment of their developability (Vlasak & Ionescu, mAbs, 2011, 3:253-263; Jarasch, J. Pharm. Sci., 2015, 104:1885-1898). The levels of the clipped species and the analytical methods used to monitor the purity and integrity are part of the critical quality attribute of the product or process (Torkashvand et al., Iranian Biomedical Journal, 2017, 21:131-141; Duval et al., Biotechnology Progress, 2012, 28:608-622).

Identification of the N-terminal sequence of an intact or cleaved protein is crucial for its biochemical and structural characterization. In conventional shotgun proteomic analysis, it is difficult to identify protein N-termini due to infrequent detection of protein N-terminal peptides. Characterization of clipping sites of a protein therapeutic by shotgun mass spectrometry is challenging due to the sequencing of most abundant peptides present in complex protein digests. Relative low stoichiometry of a clipped species can potentially lead to not detecting peptides with neo N-termini. In addition, in-solution and in-source fragmentation artifacts can potentially lead to false-positive identification of neo-N-terminal peptides sequenced by mass spectrometry.

### BRIEF SUMMARY OF THE INVENTION

There remains needs in the art for the development of methods to identify and characterize polypeptides and clipping sites on polypeptides. The present invention addresses these needs.

In one general aspect, the invention relates to a method of characterizing a polypeptide, the method comprising:
(i) labeling the polypeptide with a N-terminal labeling reagent to obtain a labeled polypeptide;
(ii) digesting the labeled polypeptide to generate a mixture comprising one or more unlabeled peptides and one or more labeled peptides;
(iii) subjecting the mixture to liquid chromatography (LC) to generate elutes of the LC;
(iv) subjecting the elutes to an electron-induced dissociation mass spectrometry, such as an electron transfer dissociation (ETD) or an electron capture dissociation (ECD) mass spectrometry to obtain a mass spectrum, such as an ETD or ECD mass spectrum, of each of the one or more labeled peptides;
(v) identifying each of the one or more labeled peptides by detecting a reporter ion in the mass spectrum, such the ETD or ECD mass spectrum, of each of the one or more labeled peptides;
(vi) subjecting each of the identified labeled peptides to a second mass spectrometry to thereby generate a second mass spectrum of each of the labeled peptides; and
(vii) characterizing the polypeptide by analyzing the ETD or ECD mass spectrum and the second mass spectrum of each of the labeled peptides.

In some embodiments, the N-terminal labeling reagent is N-tris(2,4,6-trimethoxyphenyl)phosphonium acetyl (TMPP). In further embodiments, the labeled polypeptide is a TMPP labeled polypeptide and the labeled peptide is a TMPP labeled peptide.

In some embodiments, the reporter ion is a TMPP reporter ion. In further embodiments, the TMPP reporter ion has a nominal mass-to-charge (m/z) of about 533 Da, about 573 Da, or about 590 Da.

In some embodiments, the second mass spectrometry is collision-induced dissociation (CID) mass spectrometry, higher-energy collisional dissociation (HCD) mass spectrometry, or ultra-violet photodissociation (UVPD) mass spectrometry. In further embodiments, the second mass spectrum of the labeled peptide is a CID, HCD, or UVPD mass spectrum of the TMPP labeled peptide.

In certain embodiments, the TMPP reporter ion triggers the CID mass spectrometry, the HCD mass spectrometry, or the UVPD mass spectrometry.

In another general aspect, the invention relates to a method of identifying a clipping site on a protein, the method comprising:
(i) obtaining a sample containing one or more clipped polypeptide of the protein;
(ii) labeling the one or more clipped polypeptides with a N-terminal labeling reagent to obtain one or more labeled clipped polypeptides;
(iii) digesting the labeled clipped polypeptides to generate a mixture comprising unlabeled peptides and labeled peptides;
(iv) subjecting the mixture to liquid chromatography (LC) to generate elutes of the LC;
(v) subjecting the elutes to an electron-induced dissociation tandem mass spectrometry, such as an electron transfer dissociation (ETD) or an electron capture dissociation (ECD) mass spectrometry to obtain a mass spectrum, such as an ETD or ECD mass spectrum, of each of the labeled peptides;
(vi) identifying the labeled peptide by detecting a reporter ion in the mass spectrum, such as ETD or ECD mass spectrum, for each of the labeled peptides;
(vii) subjecting the identified labeled peptides to a second mass spectrometry to thereby generate a second mass spectrum for each of the labeled peptides; and
(viii) characterizing the polypeptide by analyzing the ETD or ECD mass spectrum and the second mass spectrum for each of the labeled peptides.

In some embodiments, the N-terminal labeling reagent is N-tris(2,4,6-trimethoxyphenyl)phosphonium acetyl (TMPP). In further embodiments, the labeled polypeptide is a TMPP labeled polypeptide and the labeled peptide is a TMPP labeled peptide.

In some embodiments, the reporter ion is a TMPP reporter ion. In further embodiments, the TMPP reporter ion has a nominal mass-to-charge (m/z) of about 533 Da, about 573 Da, or about 590 Da.

In some embodiments, the second mass spectrometry is collision-induced dissociation (CID) mass spectrometry, higher-energy collisional dissociation (HCD) mass spectrometry, or ultra-violet photodissociation (UVPD) mass spectrometry. In further embodiments, the second mass spectrum of the labeled peptide is a CID, HCD, or UVPD mass spectrum of the TMPP labeled peptide.

In certain embodiments, the TMPP reporter ion triggers the CID mass spectrometry, the HCD mass spectrometry, or the UVPD mass spectrometry.

In another general aspect, the invention relates to a method of identifying a clipping site on a protein, the method comprising:
(i) obtaining a sample containing one or more clipped polypeptides of the protein;
(ii) labeling the one or more clipped polypeptides with a N-tris(2,4,6-trimethoxyphenyl)phosphonium acetyl (TMPP) to thereby obtain one or more TMPP labeled clipped polypeptides;
(iii) digesting the one or more TMPP labeled clipped polypeptides to generate a mixture comprising unlabeled peptides and TMPP labeled peptides;
(iv) subjecting the mixture to liquid chromatography (LC) to generate elutes of the LC;
(v) subjecting the elutes to an electron-induced dissociation mass spectrometry, such as an electron transfer dissociation (ETD) or an electron capture dissociation (ECD) mass spectrometry to obtain a mass spectrum, such as an ETD or ECD mass spectrum, of each of the TMPP labeled peptides;
(vi) detecting or separating a TMPP reporter ion in the mass spectrum, such as the ETD or ECD mass spectrum, for each of the TMPP labeled peptide;
(vii) upon detection or separation of the TMPP reporter ion, subjecting each of the TMPP labeled peptides to collision-induced dissociation (CID) mass spectrometry or higher-energy collisional dissociation (HCD) mass spectrometry to thereby generate a CID or HCD mass spectrum for each of the TMPP labeled peptides, respectively; and
(viii) identifying the clipping site on the protein by analyzing the ETD or ECD mass spectrum and the CID or HCD mass spectrum for each of the TMPP labeled peptides.

In other aspects, the present invention relates, in part, to systems for identifying a clipping site on a polypeptide and/or characterizing a polypeptide in a sample. In various embodiments, the system comprises a liquid chromatography (LC) device and a tandem mass spectrometer.

In one embodiment, the tandem mass spectrometer comprises:
(i) a first ionization device;
(ii) a first mass to charge ratio filter or mass to charge ratio mass analyzer arranged and adapted in a first mode of operation to transmit ions having a mass to charge ratio within a first range;
(iii) a first ion mobility spectrometer, detector, or separator;
(iv) attenuation means for attenuating ions in a mode of operation;
(v) a control device configured to control the operation of the attenuation means so that ions having mass to charge ratios within the first range but having one or more undesired first charge states are substantially attenuated;
(vi) a second ionization device;
(vii) a second ion mobility spectrometer, detector, or separator; and
(viii) a data system configured to acquire non-mixed signals of fragment ions and to non-redundantly encode triggering ions, the non-redundant encoding being arranged to avoid or minimize repetitive overlapping of any two ion signals from different parent species at multiple repetitions of any individual gate time.

In various embodiments, the clipping site on the polypeptide or the polypeptide is labeled with N-tris(2,4,6-trimethoxyphenyl)phosphonium acetyl (TMPP). In one embodiment, the first ionization device generates a TMPP reporter ion.

In one embodiment, the sample is subject to the LC device to generate elutes. In one embodiment, the elutes are subjected to the tandem mass spectrometry to obtain a first mass spectrum and a second mass spectrum.

In some embodiments, the first mass spectrum and the second mass spectrum are analyzed by comparing with information in a database or a spectral library.

In one embodiment, the first ionization device is an electron-induced dissociation device. In some embodiments, the electron-induced dissociation device is an electron transfer dissociation (ETD) device or electron capture dissociation (ECD) device.

In some embodiments, the second ionization device is a collision-induced dissociation (CID) device, higher-energy collisional dissociation (HCD) device, or ultraviolet photodissociation (UVPD) device.

In some embodiments, the mass spectrometer further comprises a collision device, fragmentation device, or reaction device.

In some embodiments, the attenuation means comprises an ion gate or ion barrier. In one embodiment, the attenuation means is arranged downstream of the ion mobility spectrometer or separator.

In some embodiments, the first mass to charge ratio filter or mass to charge ratio mass analyzer is arranged and adapted in the first mode of operation to attenuate ions having mass to charge ratios outside of the first range. In some embodiments, the first mass to charge ratio filter or mass to charge ratio mass analyzer is arranged upstream or downstream of said ion mobility spectrometer or separator.

In some embodiments, the first undesired charge state is selected from one or more of the following: (i) singly charged; (ii) doubly charged; (iii) triply charged; (iv) quadruply charged; (v) quintuply; and (vi) multiply charged.

In some embodiments, the system further comprises an ion guide, ion trap or ion trapping region arranged upstream of said ion mobility spectrometer or separator, wherein said ion guide, ion trap or ion trapping region is arranged to trap, store or accumulate ions and then to periodically pulse ions into or towards said ion mobility spectrometer or separator.

In other aspects, the present invention relates, in part, to reporter ions for identifying a clipping site on a polypeptide. In one embodiment, the clipping site is labeled with N-tris(2,4,6-trimethoxyphenyl)phosphonium acetyl (TMPP). In one embodiment, the TMPP is ionized to generate the reporter ion.

In other aspects, the present invention relates, in part, to reporter ions for characterizing a polypeptide. In one embodiment, the polypeptide is labeled with N-tris(2,4,6-trimethoxyphenyl)phosphonium acetyl (TMPP). In one embodiment, the TMPP is ionized to generate the reporter ion.

In one embodiment, the TMPP is ionized by a mass spectrometer to generate the reporter ion. In one embodiment, the mass spectrometer is a tandem mass spectrometer. In various embodiments, the tandem mass spectrometer comprises an electron transfer dissociation (ETD) device, electron capture dissociation (ECD) device, collision-induced dissociation (CID) device, higher-energy collisional dissociation (HCD) device, ultraviolet photodissociation (UVPD) device, or any combination thereof.

In some embodiments, the reporter ion has a nominal mass-to-charge (m/z) of about 533 Da, about 573 Da, or about 590 Da.

In some embodiments, the reporter ion is a compound having a structure of:

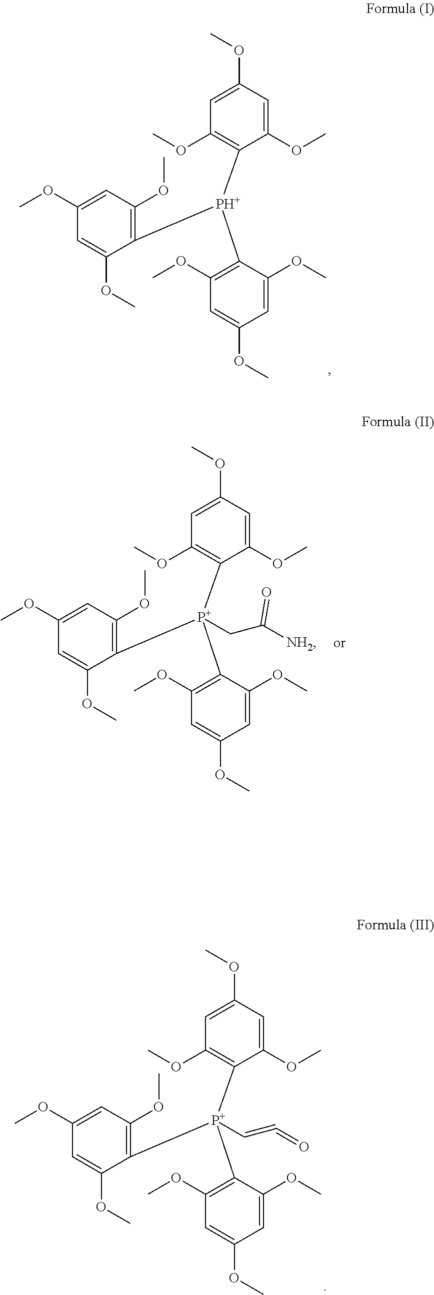

In other aspects, the present invention relates, in part, to compositions for identifying a clipping site on a polypeptide.

In other aspects, the present invention relates, in part, to compositions for characterizing a polypeptide

In various embodiments, the composition comprises at least one reporter ion of the present invention and a polypeptide.

In other aspects, the present invention also relates, in part, to kits for identifying a clipping site on a polypeptide or characterizing a polypeptide in a sample, the kit comprising: N-tris(2,4,6-trimethoxyphenyl)phosphonium acetyl (TMPP) for labeling the clipping site on the polypeptide or N-tris(2,4,6-trimethoxyphenyl)phosphonium acetyl (TMPP) for labeling the polypeptide; and an instructional material.

Other aspects, features and advantages of the invention will be apparent from the following disclosure, including the detailed description of the invention and its preferred embodiments and the appended claims.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts representative ETD product ion spectrum of the TMPP labeled peptide DIQMTQSPSTL (SEQ ID NO: 1) corresponding to light chain N-terminal sequence of the NIST antibody. The mass spectrum consisted predominantly of the diagnostic TMPP reporter ion (m/z=533 Da) and of c-type backbone product ions that consisted the N-terminal TMPP tag (m/z=590 Da).

FIGS. 2A-C depict schematic representation of structures of the reporter ions at 533, 590, and 573 Da with their exact mass: FIG. 2A depicts schematic representation of structures of the reporter ion of (TMPP)⁺ at 533 Da, FIG. 2B depicts schematic representation of structures of the reporter ion of (TMPP-Ac-NH₂)⁺ at 590 Da, and FIG. 2C depicts schematic representation of structures of the reporter ion of (TMPP-Ac)⁺ at 573 Da. FIGS. 2D-E show the formulas for calculating the efficiency of generating reporter ions in ETD (FIG. 2D) and HCD (FIG. 2E).

FIG. 3 depicts representative reverse-phase chromatographic buffer gradient and the corresponding total ion chromatogram of NIST digest after TMPP labeling. Most unlabeled peptides eluted at 2˜30% organic in a 10 minutes long shallow gradient. The surrogate peptides corresponded to the TMPP labeled N-termini of the NIST antibody light chain eluted at 12 min.

FIGS. 4A-Z depicts representative ETD-MS² and triggered CID-MS² spectra of twenty TMPP labeled synthetic peptides, including HK (FIGS. 4A and 4B), ADYEK (SEQ ID NO: 12) (FIG. 4C), VYACEVTHQGLSSPVTK (SEQ ID NO:13) (FIG. 4D), SFNR (SEQ ID NO: 14) (FIG. 4E), EAK (FIG. 4F), VQWK (SEQ ID NO: 15) (FIG. 4G), DTLMISR (SEQ ID NO: 16) (FIG. 4H), FNWYVDGVEVHNAK (SEQ ID NO: 17) (FIG. 4I), TKPR (SEQ ID NO: 18) (FIG. 4J), EEQYNSTYR (SEQ ID NO: 19) (FIG. 4K), VVSVLTVLHQDWLNGK (SEQ ID NO: 20) (FIG. 4L), EYK (FIG. 4M), CK (FIG. 4N), GQPR (SEQ ID NO: 21) (FIG. 4O), EPQVYTLPPSR (SEQ ID NO: 22) (FIGS. 4P-4R), STSGGTAALGCLVKD (SEQ ID NO: 23) (FIGS. 4S-4U), STSGGTAALGCLVKDYFPEPVTVSWN (SEQ ID NO: 24) (FIG. 4V), VVSLTVLHQDWLNGKE (SEQ ID NO: 25) (FIGS. 4W-4X), VVSLTVLHQDWLNGK (SEQ ID NO: 26) (FIG. 4Y), and VSLTVLHQDWLNGK (SEQ ID NO: 27) (FIG. 4Z).

FIG. 5A depicts representative results demonstrating peptide intensities as a function of observed retention times overlaid with the AcCN gradient, and FIG. 5B depicts representative results demonstrating peptide intensities as a function of observed retention times overlaid with the AcCN gradient.

FIG. 6A depicts representative results demonstrating TMPP⁺ ETD efficiency as a function of mass. FIG. 6B depicts representative results demonstrating TMPP⁺ ETD efficiency as a function of sequence length. FIG. 6C depicts representative results demonstrating TMPP⁺ ETD efficiency as a function of charge. FIG. 6D depicts representative results demonstrating intensity difference as the efficiency of 533 and 590 diagnostic ions, which were TMPP⁺ and TMPP-Ac-NH₂⁺, respectively. For the same peptides, smaller efficiency values (single digit) were observed for 590 diagnostic ion compared to 533 diagnostic ion (double digit efficiency values). Accordingly, the intensity of 533 diagnostic ion was significantly higher than 590 diagnostic ion.

FIG. 7A-7D depicts representative results demonstrating that overall ETD efficiency, considering all product ions, showed a subtle charge state dependent decrease. The ETD efficiency can be defined by Equations shown in FIG. 2D. The Original % ETD Efficiency as defined by Gunawardena et al. (Gunawardena H P et al., 2005, Journal of the American Chemical Society, 127:12627-12639) was Eq5, which was the overall ETD efficiency estimate for all backbone fragments of a polypeptide (reported as a percentage). Eq1-3 were derived as an estimate for ETD efficiency specific to the reporter ion. Eq4 was ETD efficiency estimate for reporter ions as well as backbone fragments of the polypeptide. In contrast, FIG. 7C and FIG. 7D depicts representative results demonstrating that, for overall backbone efficiency estimated by Eq5 where TMPP⁺ reporter ions were disregarded, the ETD efficiency showed a charge state dependent increase. FIG. 7A depicts representative results demonstrating overall ETD efficiency as a function of mass using Eq4. FIG. 7B depicts representative results demonstrating overall ETD efficiency as a function of charge using Eq4. FIG. 7C depicts representative results demonstrating overall ETD efficiency as a function of mass using Eq5. FIG. 7D depicts representative results demonstrating overall ETD efficiency as a function of charge using Eq5. FIG. 7E depicts representative results demonstrating TMPP⁺ HCD efficiency as a function of mass using Eq6. FIG. 7F depicts representative results demonstrating TMPP⁺ HCD efficiency as a function of charge using Eq6.

FIG. 8 depicts representative results demonstrating the relationship between the ETD efficiency and the labeled peptides grouped by the number tyrosine and lysine residues per peptide.

FIG. 9A depicts representative results demonstrating the overall distribution of the reaction or TMPP labeling efficiency of peptides. FIG. 9B depicts representative results demonstrating the overall distribution of the reaction or TMPP labeling efficiency of peptides estimated by Eq7.

FIGS. 10A-C depict representative results demonstrating the generation of True Positive (TP), False Positive (FP), True Negative (TN), and False Negative (FN) in labelled peptides and unlabeled peptides. As indicated in FIG. 10A, the labeled peptide produced a characteristic TMPP⁺ reporter ion, which was a True Positive (TP), while the unlabeled peptide counterpart did not produce a reporter, which was a True Negative (TN). As indicated in FIG. 10B, the labeled peptide produced a characteristic TMPP⁺ reporter ion, which was a True Positive (TP), while the unlabeled peptide counterpart produced an interfering ion similar in mass to the TMPP⁺ reporter ion, which was a False Positive (FP). As indicated in FIG. 10C, the modified peptide did not produce a diagnostic ion, which was a False Negative (FN), while the unmodified peptide counterpart produced an interfering ion similar in mass to the TMPP⁺ reporter ion, which was a False Positive (FP).

FIGS. 11A-D depict representative results demonstrating the Area Under the Curve (AUC) of the ROC curve for each diagnostic ion and elution time. FIG. 11A depicts representative predictive power of TMPP⁺ (533) diagnostic ion; FIG. 11B depicts representative predictive power of TMPP peptide retention time; FIG. 11C depicts representative predictive power of TMPP-Ac⁺ (573) diagnostic ion; and FIG. 11D depicts representative predictive power of TMPP-Ac-NH₂⁺ (591) diagnostic ion.

FIGS. 12A-C depict representative MS/MS spectra showing evidence for neo-N termini of the sequence of IAWLVK (SEQ ID NO: 5) generated due to protease activity. FIG. 12A depicts representative product ion spectrum resulting from ETD-MS² of the doubly charged ion produced a characteristic diagnostic ion TMPP⁺ (m/z=533 Da). FIG. 12B depicts representative diagnostic reporter ion (m/z=533) triggered CID-MS² spectrum of the doubly charged ions. FIG. 12C depicts representative ETD spectrum of the unconjugated peptide where diagnostic ions were absent.

FIGS. 13A-C depicts representative results demonstrating the evidence of ETD-MS² and diagnostic ion triggered CID-MS² product ion spectra of surrogate peptides corresponding to the sequential clipping of a GLP1 sequence. FIG. 13A depicts representative results demonstrating the surrogate peptides of AWLVK (SEQ ID NO: 6) resulting from I/A clip. FIG. 13B depicts representative results demonstrating the surrogate peptides of WLVK (SEQ ID NO: 7) resulting from a A/W clip. FIG. 13C depicts representative results demonstrating the surrogate peptides of LVK resulting from the W/L clip.

FIG. 14 depicts schematic representation of the clipping sites of dulaglutide (SEQ ID NO: 2) to generate surrogate peptides of EFIAWLVK (SEQ ID NO: 3), FIAWLVK (SEQ ID NO: 4), IAWLVK (SEQ ID NO: 5), AWLVK (SEQ ID NO: 6), WLVK (SEQ ID NO: 7), and LVK.

FIGS. 15A-B depict representative extracted ion chromatograms (XIC) of the surrogate peptides of dulaglutide. FIG. 15A depicts representative extracted ion chromatograms of surrogate peptides without TMPP labelling, including the peptides of SEQ ID NOs: 4-7 and the peptide of LVK. FIG. 15B depicts representative extracted ion chromatograms of labeled surrogate peptides, including TMPP-FIAWLVK (SEQ ID NO: 8), TMPP-IAWLVK (SEQ ID NO: 9), TMPP-AWLVK (SEQ ID NO: 10), TMPP-WLVK (SEQ ID NO: 11), and TMPP-LVK. The XIC demonstrated that each TMPP labeled peptides eluted during the two rapid ramps between 10-13 min.

FIGS. 16A-C depict representative results demonstrating the characteristic reporter ions via different dissociation modes on the LVK peptide sequence. FIG. 16A depicts representative results demonstrating the characteristic reporter ions via higher-energy collisional dissociation (HCD) on the LVK peptide sequence. FIG. 16B depicts representative results demonstrating the characteristic reporter ions via ultraviolet photodissociation (UVPD) on the LVK peptide sequence. FIG. 16C depicts representative results demonstrating the characteristic reporter ions via electron transfer dissociation (ETD) on the LVK peptide sequence.

FIGS. 17A-B depict representative results demonstrating GLP1 peptide clipping at F-I in the presence and absence of Cathepsin D and buffered solutions at different pH and buffer compositions used for TMPP derivatization. Cathepsin D treated samples were denoted as +Cathepsin D and TMPP derivatized samples are denoted as +TMPP; x-axis displays sample and reaction conditions: 100 mM MES pH 6, 100 mM HEPES pH 7, 100 mM Sodium phosphate pH 8; TMPP reagent premixed with DMF; Unreacted controls in PBS pH 7; y-axis displays peak area. FIG. 17A depicts representative panel of extracted ion chromatograms (XIC) of the precursor peptide FIAWLVK (SEQ ID NO: 4) and corresponding clipped product IAWLVK (SEQ ID NO: 5) where Y-axis displays the peak area and x-axis displays retention time. FIG. 17B depicts representative peak areas of XICs for precursor peptide FIAWLVK (SEQ ID NO: 4) and corresponding clipped product IAWLVK (SEQ ID NO: 5).

### DETAILED DESCRIPTION OF THE INVENTION

The present invention is based, in part, on the unexpected discovery that the use of TMPP labeling in conjunction with electron transfer dissociation (ETD) mass spectrometry generated facile TMPP⁺ reporter ions that were most intense for small tryptic peptides. Additionally, the present invention is based, in part, on the unexpected discovery that the collision-induced dissociation (CID)-MS² spectra complimented the ETD identifications and triggered MS² scans, providing a real time in silco filtering mechanism where a CID scan was only performed when the reporter ion was observed.

Thus, the present invention relates, in part, to novel systems, processes, and methods for characterizing a protein or a polypeptide and/or identifying clipping sites on a protein or a polypeptide. In various embodiments, the systems, processes, and methods comprise high-throughput LC-MS for the facile generation of reporter ions upon ETD. In some embodiments, the generation of the reporters facilitates subsequent MS/MS analysis. In some embodiments, the MS/MS analysis comprises complementary ion activation modes, such as CID, high-energy collision dissociation (HCD), and/or ultraviolet photodissociation (UVPD), via intensity and m/z dependent triggering events to further sequence the proteins or polypeptides. For example, in one embodiment, the present invention focuses on a method of identifying clipped polypeptides, comprising ETD-MS2, wherein TMPP derived reporter-ions trigger a MS2 analysis to autonomously filter clipped polypeptides.

### Definitions

As used herein, each of the following terms has the meaning associated with it in this section. Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents, published patent applications, and publications cited herein are incorporated by reference as if set forth fully herein. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

The articles “a” and “an” are used herein to refer to one or to more than one (i.e. to at least one) of the grammatical object of the article. By way of example, “an element” means one element or more than one element.

Unless otherwise stated, any numerical value, such as a concentration or a concentration range described herein, are to be understood as being modified in all instances by the term “about.” Thus, a numerical value typically includes ±10% of the recited value. For example, an amount of about 50 ppm or less includes 45 ppm or less to 55 ppm or less. As used herein, the use of a numerical range expressly includes all possible subranges, all individual numerical values within that range, including integers within such ranges and fractions of the values unless the context clearly indicates otherwise.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “MS/MS” or “MS²” refers to tandem mass spectrometry. Tandem mass spectrometry is a technique in instrumental analysis where two or more mass analyzers are coupled together using an additional reaction step to increase their abilities to analyze samples. Tandem use of mass analysis can be done where the reaction steps are separated in space (tandem in space) and/or reaction steps are separated in time (tandem in time). A common use of tandem mass spectrometry is the analysis of biomolecules, such as proteins, peptides, organic and inorganic molecules, lipid, metabolites, and oligonucleotides.

As used herein, “reporter ion” or “diagnostic ion” refers to a characteristic product ion of a labeled peptide or polypeptide containing an N-terminal tag or label, which is observed in the ETD mass spectrum. Usually, it is the most dominant product ion in the mass spectrum, and it is used to trigger subsequent MS/MS events to further sequence the labeled peptide or polypeptide.

The term “label” when used herein refers to a detectable compound or composition that is conjugated directly or indirectly to a probe to generate a “labeled” probe. The label may be detectable by itself (e.g., small molecule or charge labels).

The term “amplification” refers to the operation by which the number of copies of a target reporter ion present in a sample is multiplied.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, the terms “amino acid”, “amino acidic monomer”, or “amino acid residue” refer to any of the twenty naturally occurring amino acids, synthetic amino acids with unnatural side chains, and including both D and L optical isomers.

As used herein, the terms “natural amino acid”, “naturally encoded amino acid”, “naturally occurring amino acid”, and “genetically encoded amino acid” refer to an amino acid that is one of the twenty common amino acids or pyrolysine or selenocysteine. The term “natural amino acid” includes, but is not limited to, proteinogenic amino acids.

A “non-natural amino acid” refers to an amino acid that is not one of the twenty common amino acids or pyrolysine or selenocysteine. Other terms that may be used synonymously with the term “non-natural amino acid” is “non-naturally encoded amino acid,” “unnatural amino acid,” “non-naturally-occurring amino acid,” “non-genetically encoded amino acid”, and variously hyphenated and non-hyphenated versions thereof. The term “non-natural amino acid” includes, but is not limited to, amino acids which occur naturally by modification of a naturally encoded amino acid (including but not limited to, the common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, nonproteinogenic amino acids and amino acids, which do not occur naturally and may be obtained synthetically (e.g., Q-proline-based amino acids) or may be obtained by modification of non-natural amino acids.

“Isolated” means altered or removed from the natural state. For example, a protein or a peptide naturally present in a living animal is not “isolated,” but the same protein or peptide partially or completely separated from the coexisting materials of its natural state is “isolated.” An isolated peptide or protein can exist in substantially purified form, or can exist in a non-native environment such as, for example, a host cell.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

“Instructional material,” as that term is used herein, includes a publication, a recording, a diagram, or any other medium of expression which can be used to communicate the usefulness of the reporter ion, system and/or method of the invention in the kit for identifying clipping site on a polypeptide or characterizing a polypeptide. Optionally, or alternately, the instructional material may describe one or more methods of labeling the polypeptide or the clipping site of the polypeptide with TMPP. Optionally, or alternately, the instructional material may describe one or more methods of analyzing the TMPP-labeled clipping site on the polypeptide or TMPP-labeled polypeptide using the systems or methods of the invention. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates, in part, to novel systems, processes, and methods for characterizing a protein or a polypeptide and/or identifying clipping sites on a protein or a polypeptide. In various aspects of the invention, the systems, processes, and methods comprise high-throughput LC-MS for the facile generation of reporter ions upon ETD. In some embodiments, the generation of the reporters facilitates subsequent MS/MS analysis (i.e., tandem mass spectrometry). In some embodiments, the MS/MS analysis comprises complementary ion activation modes, such as CID, high-energy collision dissociation (HCD), and/or ultraviolet photodissociation (UVPD), via intensity and m/z dependent triggering events to further sequence the proteins or polypeptides. For example, in one embodiment, the present invention focuses on a method of identifying clipped polypeptides, comprising ETD-MS2, wherein TMPP derived reporter-ions trigger a MS2 analysis to autonomously filter clipped polypeptides.

### Identification of Clipping Sites

Mass spectrometry is an important emerging method for clipping sites identification and characterization. In keeping with the performance and mass range of available mass spectrometers, two approaches are used for characterizing proteins including “top-down” strategy and “bottom-up” strategy.

In “top-down” strategy of protein analysis, intact proteins are ionized by either electrospray ionization (ESI) or matrix-assisted laser desorption/ionization (MALDI), and then introduced to a mass analyzer. However, intact MS is only useful at detecting degradation products that are within the instrument's limit of detection (LOD), while low-levels clips are often unobserved in intact MS analysis, which requires peptide level analysis.

In “bottom-up” proteomics, identification of the existence of proteins is at the peptide level. A common procedure of “bottom-up” strategy involves using one or more proteolytic enzymes (such as typsin, pepsin, chymotrypsin, etc.) to obtain masses of individual peptides derived from the protein. Subsequently these peptides are introduced into the mass spectrometer and identified by peptide mass fingerprinting or tandem mass spectrometry. Then the masses are compared against a database such as sequence database or spectral library, and probability-based scoring systems are used to determine the closest protein matches. Hence, this approach uses identification at the peptide level to infer the existence of clipped peptides. The smaller and more uniform fragments are easier to analyze than intact proteins and can also be determined with high accuracy, this “bottom-up” approach is therefore the preferred method of studies in proteomics and protein characterization.

However, characterization of clipping sites of a protein by the bottom-up approach is challenging due to the sequencing of most abundant peptides present in complex protein digests. Relatively low stoichiometry of a clipped peptide can potentially lead to not detecting peptides with neo-N-termini. In addition, in-solution and in-source fragmentation artifacts can potentially lead to false-positive identification of neo-N-terminal peptides sequenced by mass spectrometry.

To increase the confidence of protein N-terminal identification, chemical derivatization of the N-terminal amine group by (N-Succinimidyloxycarbonylmethyl)tris(2,4,6-trimethoxyphenyl)phosphonium bromide (TMPP) or dimethyl labeling followed by mass spectrometric analysis is commonly performed. Using this approach, the protein N-terminus of interest is labeled by TMPP or dimethyl before tryptic digestion and LC-MS analysis. The N-terminus of the protein can thus be easily identified because only the N-terminal tryptic peptide contains the labeling. Peptides with N-terminal derivatization such as TMPP improves ionization and retention of peptides during chromatography and produces unique fragment ions during tandem mass spectrometric analysis, which significantly facilitates sequencing of these peptides.

One method based on chemical labeling is demethylation, which labels peptide N-termini and 8-amino groups of lysine with water-soluble formaldehyde via reductive methylation. In MS/MS analysis, this labeling strategy provides a signal enhancement for the al and yn-1 ions, which are not detectable from most of the nonderivatized fragments. Because of its simplicity, as well as low cost, dimethyl labeling is a promising strategy for protein N-termini identification.

Alternatively, the TMPP labeling approach is straightforward and has been successfully applied to different proteins. Two characteristics of this labeling reagent promote the sensitivity of the method: (i) TMPP labeling introduces a permanent positive charge resulting in an enhanced ionization efficiency and thus a better detection of low-abundance peptides; (ii) the hydrophobic TMPP group shifts the retention time of TMPP derivatized peptides in reversed phase chromatography toward a less complex part of the chromatogram, increasing the sensitivity of detection, especially for short N-terminal peptides that otherwise would not be retained on the column. In addition, TMPP is fully compatible with all standard detergents, chaotropic agents, and reduction conditions used for protein extraction in proteomics, which makes TMPP labeling a commonly used method for protein N-terminal sequencing.

The TMPP labeling approach has been demonstrated for detecting protein clipping from proteins excised from SDS-PAGE gels and proteogenomic mapping of N-terminal heterogeneity. In addition, this labelling reagent increases the hydrophobic properties of the N-terminal peptides, improves their ionization ability, and modifies their fragmentation pattern due to the positive charge introduced.

### Reporter Ion Triggered Tandem Mass Spectrometry

The site-specific localization of the TMPP tag allows for unambiguous determination of the mature N-termini or neo N-termini. In addition to backbone product ions, TMPP reporter ions at 273 Da formed via collision induced dissociation (CID) can be diagnostic for the presence of a processed N-termini. However, reporter ions generated through CID may be less informative due to their lower abundance.

The present application describes a novel high-throughput LC-MS method for the facile generation of TMPP reporter ion upon electron transfer dissociation (ETD) tandem mass spectrometry. The abundant generation of these reporter ions allows for subsequent MS/MS event using complementary ion activation modes such as CID, HCD or UVPD via intensity and m/z dependent triggering events to further sequence peptides. The reporter ion generated via ETD is novel, and triggering of this reporter facilitates the filtering of spectra that contain TMPP labeled peptides, assists in database searches, or rapid manual validation of spectra.

In one general aspect, the present application relates to a method of characterizing a polypeptide, the method comprising:
(i) labeling the polypeptide with a N-terminal labeling reagent to obtain a labeled polypeptide;
(ii) digesting the labeled polypeptide to generate a mixture comprising one or more unlabeled peptides and one or more labeled peptides;
(iii) subjecting the mixture to liquid chromatography (LC) to generate elutes of the LC;
(iv) subjecting the elutes to electron transfer dissociation (ETD) mass spectrometry;
(v) identifying the one or more labeled peptides by detecting a reporter ion in an ETD mass spectrum of the one or more labeled peptide;
(vi) subjecting the identified one or more labeled peptides to a second mass spectrometry to thereby generate a second mass spectrum of each of the labeled peptides; and
(vii) characterizing the polypeptide by analyzing the ETD mass spectrum and the second mass spectrum of each of the labeled peptides.

According to the embodiments of the present application, the polypeptide to be characterized can be a fragment (clipped polypeptide) resulting from clipping of a protein or peptide, including but not limited to, an enzyme, an antibody (e.g., a monoclonal antibody, bi-specific antibody, tri-specific antibody, tetra-specific antibody) or antigen binding fragment thereof, a biomolecular antigen, a fusion protein, a fusion-peptide, a scaffold protein or peptide, a protein or peptide drug conjugate, or any other polypeptide or peptide useful as a therapeutic or diagnostic modality. The polypeptide itself can have one or more clipped sites and thus can be clipped to generate clipped peptides.

The N-terminal labelling reagent can react with the terminal amine groups, including any primary amine reactive reagents. Examples of the reagents include, but are not limited to, Sanger's reagent, dansyl derivatives, phenyl isothiocyanate (PITC), dimethoxy pyrimidine-2-isothiocyanate (DMPITC), N-hydroxysuccinimide (NHS) reagent, (N-Succinimidyloxycarbonylmethyl)tris(2,4,6-trimethoxyphenyl)phosphonium bromide (TMPP), dimethyl labeling reagents, tandem mass tags (TMT), and isobaric tags for relative and absolute quantitation (iTRAQ).

The N-terminal labelling reagents suitable for the present invention are fixed charge derivatizing reagent. Examples of these reagents include, but not limited to, TMPP, TMT, or iTRAQ. These reagents can add a tag with fixed positive charge to the peptide. The fixed charge tag result in better ionization and the hydrophobicity of the tag result in greater retention in reversed-phase chromatography. Furthermore, subsequent fragmentation of fixed charge peptides in the mass spectrometry result in a variety of backbone fragments and charged tag fragments, including reporter ions, which can be diagnostic in nature and facilitate in peptide identification. In particular, a reporter ion is generated from a charge loss upon fragmentation.

Some of these reagents are also characteristic with steric bulk to improve the reaction specificity towards free N-termini and not any other free amine such as lysine.

In some embodiments, the N-terminal labelling reagent is TMPP. In further embodiments, the labeled polypeptide is a TMPP labeled polypeptide and the labeled peptide is a TMPP labeled peptide.

In some embodiments, the TMPP labeling of peptides are mostly at N-termini and additional unlabeled lysine or tyrosine residues have no effect on diagnostic TMPP⁺ ion.

In some embodiments, the enzyme used for digesting the peptide comprises any proteolytic enzyme that is known in the art.

In certain embodiments, the TMPP labeled polypeptide is digested to generate a mixture comprising an unlabeled peptide and a TMPP labeled peptide. When this mixture is subjected to liquid chromatography (LC) to generate elutes of the LC, the method allows a rapid separation of TMPP labeled peptides from the unlabeled peptides in the mixture, because TMPP labels are hydrophobic and elute later in the reversed phase gradient. This in turn allows retention time predictability of TMPP labeled peptides and further improves the specificity and reduces false-positive identification of peptides.

In some embodiments, the liquid chromatography (LC) separation step iii) can be omitted so that there is no liquid chromatography (LC) for the separation. Accordingly, in the subsequent step iv), the mixture comprising one or more unlabeled peptides and one or more labeled peptides is subjected directly to a tandem mass spectrometry via direct infusion or flow-injection. For example, in one embodiment, the tandem mass spectrometry comprises an electron transfer dissociation (ETD). In general, ETD is a type of electron-induced dissociation methods, therefore, other types of electron-based dissociation methods, such as electron capture dissociation (ECD), can also be used herein as an alternative to ETD. In some embodiments, other dissociation methods, such as high-energy collision dissociation (HCD) can also be used herein as an alternative to ETD.

According to embodiments of the application, the derivatized precursor ion subjected to ETD has no bearing on the levels of derivatization of the peptide. For example, a peptide derivatized a 100% can have the same ETD efficiency as the same peptide derivatized by 1%. This is important when considering these reactions in the context of clip site identification of proteins, derivatization efficiency at the protein has no consequence on the ETD efficiency of the surrogate peptide.

In some embodiments, the reporter ion is a TMPP reporter ion. In further embodiments, the TMPP reporter ion has a nominal mass-to-charge (m/z) of about 533 Da, about 573 Da, or about 590 Da. The nominal mass for an element is the mass number of its most abundant naturally occurring stable isotope, and for an ion or molecule, the nominal mass is the sum of the nominal masses of the constituent atoms. The accuracy of nominal mass is often good to 10 ppm.

Preferably, the inventive method allows for facile generation of TMPP reporter ion at m/z about 533 Da (TMPP⁺) and in some instances about 590 Da (TMPP-Ac-NH₂⁺) upon electron transfer dissociation (ETD), or TMPP reporter ion at m/z about 573 Da (TMPP-Ac⁺) upon high-energy collision dissociation (HCD). Usually the reporter ion at about 533 Da is the most dominant product ion. The intense product ion can be used to trigger subsequent MS/MS events to further sequence peptides. A threshold can be used to trigger the subsequent MS/MS events, including mass m/z threshold and intensity threshold. For example, a filter (mass tolerance) is often used to trigger with certain specificity as an m/z threshold. In certain embodiments, the filter is set upon a base peak of precise mass to charge at 533.1935 or 590.2150 Da per charge or Thomson with mass tolerance ranging between 1-20 ppm for triggering. The mass tolerance can be 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, or any number between therefore, preferably 5 ppm.

Alternatively, an intensity of the reporter ion can be used as a threshold, which can be set up by using instrumentation software. For example, the intensity can be set at any specific number, e.g., 10% of the base peak, and then any reporter ion with an intensity of 10% or larger than 10% of the base peak can trigger the subsequent MS/MS events.

In a preferred embodiment, the method comprises identifying the TMPP labeled peptide by detecting or separating a TMPP reporter ion in an ETD mass spectrum of the labeled peptide.

According to embodiments of the application, in general, the propensity to produce TMPP⁺ reporter ions by ETD favors doubly charged precursor over triply charged precursor ions for peptides with similar mass or same number of amino acids. Therefore, the diagnostic utility of ETD generated TMPP⁺ ions is perfectly suited to tryptic peptides which are mostly doubly charged.

In some embodiments, the second mass spectrometry is collision-induced dissociation (CID) mass spectrometry, higher-energy collisional dissociation (HCD) mass spectrometry, or ultraviolet photodissociation (UVPD) mass spectrometry. Thus, in some embodiments, the tandem mass spectrometry is collision-induced dissociation (CID) mass spectrometry (CID-MS²), higher-energy collisional dissociation tandem mass spectrometry (HCD-MS²), or ultraviolet photodissociation tandem mass spectrometry (UVPD-MS²).

In some embodiments, the second mass spectrum is a CID, HCD, or UVPD mass spectrum of the TMPP labeled peptide.

In some embodiments, the TMPP reporter ion triggers the CID, HCD, or UVPD mass spectrometry. The approach of triggered mass spectrometry makes the filtering of the data amenable to manual inspection due to the low occurrence of trigger MS² that confirm the presence of reporter ions generated from the first ETD mass spectrometry portion of the MS². Therefore, this eliminates the need for in-slico approaches or manual inspection of the ETD mass spectra that have the reporter ion peaks.

In some embodiments, the TMPP reporter ion is generated from a charge loss.

In some embodiments, the LC is high performance liquid chromatogram (HPLC) or ultra performance liquid chromatogram (UPLC).

In some embodiments, the method is high-throughput.

In some embodiments, the ETD mass spectrum (i.e., the first mass spectrum) and the second mass spectrum, such as CID, HCD, or UVPD mass spectrum, are analyzed by comparing with information in a sequence database or a spectral library.

In a preferred embodiment, the identified TMPP labeled peptide is subjected to the CID mass spectrometry.

According to embodiments of the application, TMPP labeling can also occur on lysine and tyrosine residues of the peptides, and when subjecting to ETD, the peptides carrying these TMPP modifications are can also generate diagnostic reporter ions and hence trigger CID-MS² events. These CID spectra are false positive identifications of reporters. Nevertheless, subsequent examination of the sequence ions in ETD and diagnostic ion triggered CID spectra can site-specifically localize TMPP on the sequence and help in the elimination of false-positives. For example, the subsequent triggered CID-MS² spectra can provide information of backbone ions which can be used to determine whether the TMPP moiety is assigned to the N-terminus or side chain of lysine or tyrosine. The complementary nature of ETD-MS² and triggered CID-MS² can help to rapidly screen potential clipped species irrespective of the amino acid sequence of a surrogate proteolytic peptide containing the TMPP moiety. The ability to generate TMPP⁺ ions for triggered CID-MS² presents the complete interrogation of the sequence for accurate localization of the TMPP moiety or confirmation of the sequence with high confidence.

In a preferred embodiment, the present application relates to a method of characterizing a N-tris(2,4,6-trimethoxyphenyl)phosphonium acetyl (TMPP) labeled peptide in a sample, comprising:
(i) subjecting the sample to tandem mass spectrometry comprising electron transfer dissociation (ETD);
(ii) identifying the TMPP labeled peptide by detecting or separating a TMPP reporter ion in an ETD mass spectrum of the TMPP labeled peptide;
(iii) subjecting the identified TMPP labeled peptide to a second mass spectrometry to thereby generate a second mass spectrum of the TMPP labeled peptide; and
(iv) characterizing the TMPP labeled peptide by analyzing the ETD mass spectrum and the second mass spectrum.

It is noted that the N-terminal labelling reagent, preferably TMPP, can also bind to amine groups in the side chain such as lysine and tyrosine. When TMPP labeled peptides are subjected to ETD also generates reporter ions and hence trigger the second mass spectrometry, such as CID-MS², these second mass spectra are false positive identification of reporter ions. However, careful examination of the sequence ions in ETD spectrum and reporter ion triggered CID spectrum can site-specifically localize TMPP on the sequence and help the elimination of false-positives.

In another general aspect, the present application also relates to a method of identifying a clipping site on a protein, the method comprising:
(i) obtaining a sample containing one or more clipped polypeptide of the protein; (ii) labeling the one or polypeptides with a N-terminal labeling reagent to obtain one or more labeled polypeptide;
(iii) digesting the labeled clipped polypeptides to generate a mixture comprising unlabeled peptides and labeled peptides;
(iv) subjecting the mixture to liquid chromatography (LC) to generate elutes of the LC;
(v) subjecting the elutes to tandem mass spectrometry, comprising electron transfer dissociation (ETD);
(vi) identifying the labeled peptide by detecting a reporter ion in ETD mass spectrum for each of the labeled peptides;
(vii) subjecting the identified labeled peptides to a second mass spectrometry to thereby generate a second mass spectrum for each of the labeled peptides; and
(viii) characterizing the polypeptide by analyzing the ETD mass spectrum and the second mass spectrum for each of the labeled peptides.

As used herein, the term “protein,” encompasses natural protein, synthetic protein, recombinant protein, or peptides thereof. Examples of proteins that can be analyzed by a method of the invention include, but are not limited to, an enzyme, an antibody (e.g., a monoclonal antibody, bi-specific antibody, tri-specific antibody, tetra-specific antibody) or antigen binding fragment thereof, a biomolecular antigen, a fusion protein, a fusion-peptide, a scaffold protein or peptide, a protein or peptide drug conjugate, or any other polypeptide or peptide useful as a therapeutic or diagnostic modality.

According to the embodiments of the present application, the clipped polypeptide to be characterized can be a fragment resulting from clipping of a protein such as enzyme, antibody, and biomolecular antigen. The polypeptide itself can have one or more clipped sites and thus can be clipped to generate clipped peptides.

In some embodiments, the N-terminal labeling reagent is N-tris(2,4,6-trimethoxyphenyl)phosphonium acetyl (TMPP). In further embodiments, the labeled polypeptide is a TMPP labeled polypeptide and the labeled peptide is a TMPP labeled peptide.

In some embodiments, the N-terminal labelling reagent is TMPP. In further embodiments, the labeled polypeptide is a TMPP labeled polypeptide and the labeled peptide is a TMPP labeled peptide.

In some embodiments, the step iv) can be absent so that there is no liquid chromatography (LC) for the separation. Accordingly, in the subsequent step v), the mixture comprising unlabeled peptides and labeled peptides is subjected directly to electron transfer dissociation (ETD) or electron capture dissociation (ECD) or other electron-induced dissociation tandem mass spectrometry via direct infusion or flow-injection.

In some embodiments, the reporter ion is a TMPP reporter ion. In further embodiments, the TMPP reporter ion has a nominal mass-to-charge (m/z) of about 533 Da, about 573 Da, or about 590 Da.

In a preferred embodiment, the method comprises identifying the TMPP labeled peptide by detecting or separating a TMPP reporter ion in an ETD mass spectrum of the labeled peptide.

In some embodiments, the second mass spectrometry is collision-induced dissociation (CID) mass spectrometry, higher-energy collisional dissociation (HCD) mass spectrometry, or ultraviolet photodissociation (UVPD) mass spectrometry. Thus, in some embodiments, the tandem mass spectrometry is collision-induced dissociation (CID) mass spectrometry (CID-MS²), higher-energy collisional dissociation tandem mass spectrometry (HCD-MS²), or ultraviolet photodissociation tandem mass spectrometry (UVPD-MS²).

In some embodiments, the second mass spectrum is a CID, HCD, or UVPD mass spectrum of the TMPP labeled peptide.

In some embodiments, the TMPP reporter ion triggers the CID, HCD, or UVPD mass spectrometry.

In some embodiments, the TMPP reporter ion is generated from a charge loss.

In some embodiments, the LC is high performance liquid chromatogram (HPLC) or ultra-performance liquid chromatogram (UPLC).

In one embodiment, the protein is a therapeutic protein. In one embodiment, the protein is a non-therapeutic protein.

In some embodiments, the method is high-throughput.

In some embodiments, the ETD mass spectrum and the second mass spectrum, such as CID, HCD, or UVPD mass spectrum, are analyzed by comparing with information in a database or a spectral library, such as Uniprot, NIST, or Spectra ST. However, these databases or public libraries do not have the annotated 533 or 590 reporter ions as criteria to identify, and thus are used for the identification of non-reporter ions.

In a preferred embodiment, the identified TMPP labeled peptide is subjected to the CID mass spectrometry.

In another general aspect, the invention relates to a method of identifying a clipping site on a protein, the method comprising:
(i) obtaining a sample containing one or more clipped polypeptides of the protein;
(ii) labeling the one or more clipped polypeptides with a N-tris(2,4,6-trimethoxyphenyl)phosphonium acetyl (TMPP) to thereby obtain one or more TMPP labeled clipped polypeptides;
(iii) digesting the one or more TMPP labeled clipped polypeptides to generate a mixture comprising unlabeled peptides and TMPP labeled peptides;
(iv) subjecting the mixture to liquid chromatography (LC) to generate elutes of the LC;
(v) subjecting the elutes to tandem mass spectrometry comprising electron transfer dissociation (ETD) to thereby generate an ETD mass spectrum for each of the TMPP labeled peptides;
(vi) detecting or separating a TMPP reporter ion in the ETD mass spectrum;
(vii) upon detection or separation of the TMPP reporter ion, subjecting each of the TMPP labeled peptides to collision-induced dissociation (CID) mass spectrometry or higher-energy collisional dissociation (HCD) mass spectrometry or ultraviolet photodissociation (UVPD) mass spectrometry to thereby generate a CID or HCD or UVPD mass spectrum for each of the TMPP labeled peptides, respectively; and
(viii) identifying the clipping site on the protein by analyzing the ETD mass spectrum and the CID or HCD or UPVD mass spectrum for each of the TMPP labeled peptides.

In some embodiments, the clipped polypeptide to be characterized can be a fragment resulting from clipping of a protein such as enzyme, antibody, and biomolecular antigen. The polypeptide itself can have one or more clipped sites and thus can be clipped to generate clipped peptides.

In some embodiments, the step iv) can be absent so that there is no liquid chromatography (LC) for the separation. Accordingly, in the subsequent step v), the mixture comprising unlabeled peptides and labeled peptides is subjected directly to tandem mass spectrometry comprising electron transfer dissociation (ETD) via direct infusion or flow-injection.

In some embodiments, the TMPP reporter ion has a nominal mass-to-charge (m/z) of about 533 Da, about 573 Da, or about 590 Da.

In some embodiments, ETD mass spectrum and the CID or HCD or UPVD mass spectrum are analyzed by comparing with information in a database or a spectral library.

Any suitable mass spectrum device can be used for the presently described invention in view of the present disclosure. For example, the tandem mass spectrometry, comprising ETD, can be conducted using any instrumentation that is capable of performing ETD reactions. There are a number of models and instrument vendors that can do this type dissociation, which are known in the art. For example, electron capture dissociation (ECD) bears similarities to ETD and thus can perform dissociation and produce the same reporter ion. Therefore, instrumentation that is capable of performing ECD reactions can also be used as an alternative to ETD tandem mass spectrometry. Similarly, the CID tandem mass spectrometry can be conducted using any instrumentation that is capable of performing CID reactions, including low-energy CID and high-energy CID.

Systems for Identifying a Clipping Site on a Polypeptide and/or for Characterizing a Polypeptide

The present invention also relates, in part, to systems for identifying a clipping site on a polypeptide or characterizing a polypeptide in a sample. In various aspects of the invention, the system comprises a liquid chromatography (LC) device and a tandem mass spectrometer.

In one embodiment, the LC device is a high performance liquid chromatography (HPLC) device.

In some embodiments, the tandem mass spectrometer comprises:
(i) a first ionization device;
(ii) a first mass to charge ratio filter or mass to charge ratio mass analyzer arranged and adapted in a first mode of operation to transmit ions having a mass to charge ratio within a first range;
(iii) a first ion mobility spectrometer, detector, or separator;
(iv) attenuation means for attenuating ions in a mode of operation;
(v) a control device configured to control the operation of the attenuation means so that ions having mass to charge ratios within the first range but having one or more undesired first charge states are substantially attenuated;
(vi) a second ionization device;
(vii) a second ion mobility spectrometer, detector, or separator; and
(viii) a data system configured to acquire non-mixed signals of fragment ions and to non-redundantly encode triggering ions, the non-redundant encoding being arranged to avoid or minimize repetitive overlapping of any two ion signals from different parent species at multiple repetitions of any individual gate time.

In one embodiment, the sample is subject to the LC device to generate elutes.

In one embodiment, the elutes are subjected to the tandem mass spectrometry to obtain a first mass spectrum and a second mass spectrum.

In some embodiments, the clipping site on the polypeptide or the polypeptide is labeled with a N-terminal labeling reagent. For example, in one embodiment, the N-terminal labeling reagent is N-tris(2,4,6-trimethoxyphenyl)phosphonium acetyl (TMPP).

In one embodiment, the first ionization device generates a TMPP reporter ion. In various embodiments, the TMPP reporter ion has a nominal mass-to-charge (m/z) of about 533 Da, about 573 Da, or about 590 Da. In one embodiment, the TMPP reporter ion triggers the second mass spectrometry.

In one embodiment, the first ionization device is an electron-induced dissociation device. In some embodiments, the electron-induced dissociation device is an electron transfer dissociation (ETD) device or electron capture dissociation (ECD) device. Thus, in various embodiments, the first mass spectrum is an ETD or ECD mass spectrum.

In some embodiments, the second ionization device is a collision-induced dissociation (CID) device, higher-energy collisional dissociation (HCD) device, or ultraviolet photodissociation (UVPD) device. Thus, in various embodiments, the second mass spectrum comprises a CID, HCD, or UVPD mass spectrum.

In some embodiments, the first mass spectrum and the second mass spectrum are analyzed by comparing with information in a database or a spectral library.

In some embodiments, the mass spectrometer further comprises a collision device, fragmentation device, or reaction device.

In some embodiments, the attenuation means comprises an ion gate or ion barrier. In some embodiments, the attenuation means is arranged downstream of the ion mobility spectrometer or separator.

In some embodiments, the first mass to charge ratio filter or mass to charge ratio mass analyzer is arranged and adapted in the first mode of operation to attenuate ions having mass to charge ratios outside of the first range. In some embodiments, the first mass to charge ratio filter or mass to charge ratio mass analyzer is arranged upstream or downstream of said ion mobility spectrometer or separator.

In some embodiments, the first undesired charge state is selected from one or more of the following: (i) singly charged; (ii) doubly charged; (iii) triply charged; (iv) quadruply charged; (v) quintuply; and (vi) multiply charged.

In some embodiments, the mass spectrometer further comprises an ion guide, ion trap or ion trapping region arranged upstream of said ion mobility spectrometer or separator, wherein said ion guide, ion trap or ion trapping region is arranged to trap, store or accumulate ions and then to periodically pulse ions into or towards said ion mobility spectrometer or separator.

Reporter Ions for Identifying a Clipping Site on a Polypeptide and/or for Characterizing a Polypeptide

In other aspects, the present invention also relates, in part, to a reporter ion for identifying a clipping site on a polypeptide and/or a reporter ion for characterizing a polypeptide. In one embodiment, the clipping site is labeled with an N-terminal labeling reagent. In one embodiment, the polypeptide is labeled with an N-terminal labeling reagent.

For example, in one embodiment, the N-terminal labeling reagent is N-tris(2,4,6-trimethoxyphenyl)phosphonium acetyl (TMPP) In one embodiment, the N-terminal labeling reagent is ionized to generate the reporter ion. For example, in one embodiment, the TMPP is ionized to generate the reporter ion.

In one embodiment, the N-terminal labeling reagent is ionized by a mass spectrometer to generate the reporter ion. For example, in one embodiment, the TMPP is ionized by a mass spectrometer to generate the reporter ion.

In one embodiment, the mass spectrometer is a tandem mass spectrometer. In various embodiments, the tandem mass spectrometer is any tandem mass spectrometer described herein. For example, in some embodiments, the tandem mass spectrometer comprises an electron transfer dissociation (ETD) device, electron capture dissociation (ECD) device, collision-induced dissociation (CID) device, higher-energy collisional dissociation (HCD) device, ultraviolet photodissociation (UVPD) device, or any combination thereof.

In one embodiment, the N-terminal labeling reagent is ionized by mass spectrometry technique to generate the reporter ion. For example, in one embodiment, the TMPP is ionized by mass spectrometry technique to generate the reporter ion.

In one embodiment, the mass spectrometry technique is a tandem mass spectrometry technique. In various embodiments, the tandem mass spectrometry technique is any tandem mass spectrometry technique described herein. For example, in some embodiments, the tandem mass spectrometry technique comprises an electron transfer dissociation (ETD), electron capture dissociation (ECD), collision-induced dissociation (CID), higher-energy collisional dissociation (HCD), ultraviolet photodissociation (UVPD), or any combination thereof.

In some embodiments, the reporter ion has a nominal mass-to-charge (m/z) of about 533 Da, about 573 Da, or about 590 Da.

In some embodiments, the reporter ion is a compound having a structure of:

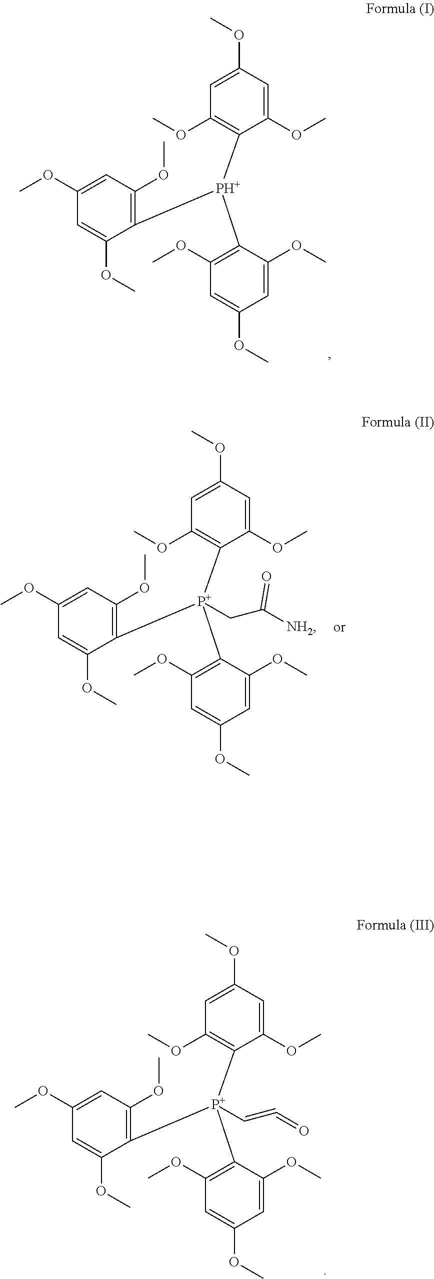

In other aspects, the present invention also relates, in part, to compositions for identifying a clipping site on a polypeptide.

In other aspects, the present invention relates, in part, to compositions for characterizing a polypeptide

In various embodiments, the composition comprises at least one reporter ion of the present invention and a polypeptide.

### Kits

The present invention also pertains to kits useful in the methods of the invention. Such kits comprise various combinations of components useful in any of the methods described elsewhere herein, including for example, materials for identifying a clipping site on a polypeptide and/or materials for characterizing a polypeptide, and instructional material. For example, in one embodiment, the kit comprises components useful for identifying a clipping site on a polypeptide in a sample. In one embodiment, the components useful for identifying a clipping site on a polypeptide in a sample comprises a TMPP. In another embodiment, the kit comprises components useful for characterizing a polypeptide in a sample. In one embodiment, the components useful for characterizing a polypeptide in a sample comprises a TMPP.

In one embodiment, the instruction material describes steps for labeling the polypeptide with TMPP. In one embodiment, the instruction material describes steps for labeling the clipping site of the polypeptide with TMPP. In some embodiments, the instructional material describes one or more methods of analyzing the TMPP-labeled clipping site on the polypeptide or TMPP-labeled polypeptide using the systems of the present invention. In some embodiments, the instructional material describes one or more methods of analyzing the TMPP-labeled clipping site on the polypeptide or TMPP-labeled polypeptide using the systems of the present invention

### Embodiments of the Invention

The invention also provides the following non-limiting embodiments.

Embodiment 1 is a method of characterizing a N-tris(2,4,6-trimethoxyphenyl)phosphonium acetyl (TMPP) labeled peptide in a sample, comprising:
(i) subjecting the sample to electron transfer dissociation (ETD) or electron capture dissociation (ECD) or other electron-based dissociation tandem mass spectrometry or higher-energy collisional dissociation (HCD) tandem mass spectrometry;
(ii) identifying the TMPP labeled peptide by detecting or separating a TMPP reporter ion in an ETD or ECD, other electron-induced dissociation mass spectrum, or HCD mass spectrum of the TMPP labeled peptide;
(iii) subjecting the identified TMPP labeled peptide to a second mass spectrometry to thereby generate a second mass spectrum of the TMPP labeled peptide; and
(iv) characterizing the TMPP labeled peptide by analyzing the ETD or ECD or other electron-induced dissociation mass spectrum or the HCD mass spectrum, and the second mass spectrum.

Embodiment 1a is the method of embodiment 1, wherein the ETD is used in the method.

Embodiment 1b is the method of embodiment 1, wherein the ECD is used in the method.

Embodiment 1c is the method of any one of embodiments 1-1b, wherein the second mass spectrometry is collision-induced dissociation (CID) mass spectrometry resulting in CID tandem mass spectrometry (CID-MS²) and the second mass spectrum is a CID mass spectrum.

Embodiment 1d is the method of any one of embodiments 1-1b, wherein the second mass spectrometry is higher-energy collisional dissociation (HCD) mass spectrometry resulting in HCD tandem mass spectrometry (HCD-MS²) and the second mass spectrum is an HCD mass spectrum.

Embodiment 1d is the method of any one of embodiments 1-1b, wherein second mass spectrometry is ultraviolet photodissociation (UVPD) mass spectrometry resulting in UPVD tandem mass spectrometry (UVPD-MS²) and the second mass spectrum is an UVPD mass spectrum.

Embodiment 2 is the method of any one of embodiments 1-1d, wherein the TMPP reporter ion has a nominal mass-to-charge (m/z) of about 533 Da or about 573 Da or about 590 Da.

Embodiment 2a is the method of embodiment 2, wherein the TMPP reporter ion has a nominal mass-to-charge (m/z) of about 533 Da.

Embodiment 2b is the method of embodiment 2a, wherein the TMPP reporter ion has an exact mass-to-charge (m/z) of 533.1935.

Embodiment 2c is the method of embodiment 2a or 2b, wherein the TMPP labeled peptide is identified by detecting a second TMPP reporter ion in the ETD or ECD or other electron-induced dissociation mass spectrum of the TMPP labeled peptide, and the second TMPP reporter ion has a nominal mass-to-charge (m/z) of about 590 Da.

Embodiment 2d is the method of embodiment 2c, wherein the second TMPP reporter ion has an exact mass-to-charge (m/z) of 590.2150.

Embodiment 2e is the method of any one of embodiments 2a-2d, wherein the TMPP labeled peptide is identified by detecting a second or third TMPP reporter ion in the ETD or ECD or HCD or other electron-induced dissociation mass spectrum of the TMPP labeled peptide, and the second or third TMPP reporter ion has a nominal mass-to-charge (m/z) of about 573 Da.

Embodiment 2f is the method of embodiment 2e, wherein the second or third TMPP reporter ion has an exact mass-to-charge (m/z) of 573.1884.

Embodiment 3 is the method of any one of embodiments 1-2f, wherein the TMPP reporter ion is generated from a charge loss.

Embodiment 3a is the method of embodiment 3, wherein the TMPP reporter ion is a predominant product ion in the ETD or ECD or other electron-induced dissociation mass spectrum.

Embodiment 3b is the method of embodiment 3a, wherein the TMPP reporter ion is a predominant product ion in the ETD mass spectrum.

Embodiment 3c is the method of embodiment 3a, wherein the TMPP reporter ion is a predominant product ion in ECD mass spectrum.

Embodiment 3d is the method of any one of the embodiments 1-3c, wherein the TMPP reporter ion is generated from a doubly charged peptide.

Embodiment 3e is the method of any one of the embodiments 1-3c, wherein the TMPP reporter ion is TMPP⁺.

Embodiment 3f is the method of any one of the embodiments 1-3c, wherein the TMPP reporter ion is TMPP-Ac-NH₂⁺.

Embodiment 3f is the method of any one of the embodiments 1-3c, wherein the TMPP reporter ion is TMPP-Ac⁺.

Embodiment 4 is the method of any one of embodiments 1-3f, wherein the TMPP reporter ion triggers the second mass spectrometry.

Embodiment 4a is the method of any one of embodiments 1-4, wherein the TMPP reporter ion triggers the second mass spectrometry via intensity threshold or m/z threshold.

Embodiment 4b is the method of embodiment 4 or 4a, wherein a filter (mass tolerance) is used to trigger the second mass spectrometry.

Embodiment 4c is the method of embodiment 4b, wherein the filter is set upon a base peak of precise mass at 533.1935, 573.1884, or 590.2150 Da with mass tolerance ranging between 1-20 ppm for triggering, such as a mass tolerance of 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, or any number between therefore, preferably 5 ppm.

Embodiment 4d is the method of embodiment 4 or 4a, wherein an intensity of the reporter ion is used as a threshold to trigger the second mass spectrometry.

Embodiment 4e is the method of embodiment 4d, wherein the intensity is set at 10% or more of the intensity of a base peak.

Embodiment 4f is the method of any one of embodiments 1-4e, wherein the TMPP reporter ion triggers the collision-induced dissociation (CID) mass spectrometry resulting in CID tandem mass spectrometry (CID-MS²).

Embodiment 4g is the method of any one of embodiments 1-4e, wherein the TMPP reporter ion triggers the higher-energy collisional dissociation (HCD) mass spectrometry resulting in HCD tandem mass spectrometry (HCD-MS²).

Embodiment 4h is the method of any one of embodiments 1-4e, wherein the TMPP reporter ion triggers the ultraviolet photodissociation UVPD mass spectrometry resulting in UVPD tandem mass spectrometry (UVPD-MS²).

Embodiment 5 is a method of characterizing a polypeptide, the method comprising:
(i) labeling the polypeptide with a N-terminal labeling reagent to obtain a labeled polypeptide;
(ii) digesting the labeled polypeptide to generate a mixture comprising one or more unlabeled peptides and one or more labeled peptides;
(iii) optionally subjecting the mixture to liquid chromatography (LC) to generate elutes of the LC;
(iv) subjecting the elutes from step iii) or the mixture from step ii) to electron transfer dissociation (ETD) or electron capture dissociation (ECD) or other electron-induced dissociation tandem mass spectrometry;
(v) identifying the labeled peptide by detecting a reporter ion in an ETD or ECD or other electron-induced dissociation mass spectrum of the labeled peptide;
(vi) subjecting the identified labeled peptide to a second mass spectrometry to thereby generate a second mass spectrum of the labeled peptide; and
(vii) characterizing the polypeptide by analyzing the ETD or ECD or other electron-induced dissociation mass spectrum and the second mass spectrum.

Embodiment 6 is the method of embodiment 5, wherein the polypeptide is a fragment polypeptide or clipped polypeptide.

Embodiment 6a is the method of embodiment 6, wherein the fragment polypeptide or clipped polypeptide results from clipping of a protein.

Embodiment 6b is the method of embodiment 6 or 6a, wherein the polypeptide has one or more clipped sites.

Embodiment 6c is the method of any one of embodiments 6-6b, wherein the protein is an enzyme, an antibody (e.g., a monoclonal antibody, bi-specific antibody, tri-specific antibody, tetra-specific antibody) or antigen binding fragment thereof, a biomolecular antigen, a fusion protein, a fusion-peptide, a scaffold protein or peptide, a protein or peptide drug conjugate, or any other polypeptide or peptide useful as a therapeutic or diagnostic modality.

Embodiment 6d is the method of embodiment 6a, wherein the protein is a therapeutic protein.

Embodiment 6e is the method of embodiment 6a, wherein the protein is a non-therapeutic protein.

Embodiment 6f is the method of any one of embodiments 5-6e, wherein the ETD is used in the method.

Embodiment 6g is the method of any one of embodiments 5-6e, wherein the ECD is used in the method.

Embodiment 7 is a method of identifying a clipping site on a protein, the method comprising:

(i) obtaining a sample containing one or more clipped polypeptide of the protein;

(ii) labeling the one or polypeptides with a N-terminal labeling reagent to obtain one or more labeled polypeptide;

(iii) digesting the labeled clipped polypeptides to generate a mixture comprising unlabeled peptides and labeled peptides;

(iv) optionally subjecting the mixture to liquid chromatography (LC) to generate elutes of the LC;

(v) subjecting the elutes from step iv) or the mixture from step iii) to electron transfer dissociation (ETD) or electron capture dissociation (ECD) or other electron-induced dissociation tandem mass spectrometry;

(vi) identifying the labeled peptide by detecting a reporter ion in an ETD or ECD or other electron-induced dissociation mass spectrum for each of the labeled peptides;

(vii) subjecting the identified labeled peptides to a second mass spectrometry to thereby generate a second mass spectrum for each of the labeled peptides; and

(viii) characterizing the polypeptide by analyzing the ETD or ECD or other electron-induced dissociation mass spectrum and the second mass spectrum.

Embodiment 8 is the method of embodiment 7, wherein the protein is an enzyme, an antibody (e.g., a monoclonal antibody, bi-specific antibody, tri-specific antibody, tetra-specific antibody) or antigen binding fragment thereof, a biomolecular antigen, a fusion protein, a fusion-peptide, a scaffold protein or peptide, a protein or peptide drug conjugate, or any other polypeptide or peptide useful as a therapeutic or diagnostic modality.

Embodiment 8a is the method of embodiment 7, wherein the protein is a therapeutic protein.

Embodiment 8b is the method of embodiment 7, wherein the protein is a non-therapeutic protein.

Embodiment 8c is the method of any one of embodiments 7-8b, wherein the clipped polypeptide of the protein has one or more clipped sites.

Embodiment 8d is the method of any one of embodiments 7-8c, wherein the ETD is used in the method.

Embodiment 8e is the method of any one of embodiments 7-8d, wherein the ECD is used in the method.

Embodiment 9 is the method of any one of embodiments 5-8e, wherein the N-terminal labeling reagent is a fixed charge derivatizing reagent.

Embodiment 9a is the method of embodiment 9, wherein the N-terminal labelling reagent adds a positive charge to the polypeptide.

Embodiment 9b is the method of embodiment 9 or 9a, wherein the N-terminal labelling reagent results in a reporter ion generated from a charge loss.

Embodiment 9c is the method of embodiment 9, wherein the N-terminal labeling reagent is TMPP.

Embodiment 9d is the method of any one of embodiments 5-9c, wherein the efficiency of the N-terminal labeling is 1%, 10%, 20%, 30%, 40%, 50%, 60%, 70%, 80%, 90%, 100%, or any number between thereof.

Embodiment 10 is the method of any one of embodiments 5-9d, wherein the labeled polypeptide is a TMPP labeled polypeptide.

Embodiment 10a is the method of embodiment 10, wherein the labeled peptide is a TMPP labeled peptide.

Embodiment 11 is the method of any one of embodiments 5-10a, wherein the LC is high performance liquid chromatogram (HPLC) or ultra-performance liquid chromatogram (UPLC), preferably HPLC.

Embodiment 11a is the method of embodiment 11, wherein the TMPP labeled peptide elutes later in a reverse phase gradient compared to the corresponding unlabeled peptide.

Embodiment 11b is the method of any one of embodiments 5-10a, wherein the mixture from step iii) is subjected to tandem mass spectrometry comprising electron capture dissociation (ECD).

Embodiment 11c is the method of embodiment 11b, wherein the mixture is subjected to the tandem mass spectrometry comprising ETD via direct infusion or flow-infusion.

Embodiment 11d is the method of any one of embodiments 5-10a, wherein the mixture from step iii) is subjected to tandem mass spectrometry comprising electron transfer dissociation (ETD).

Embodiment 11e is the method of embodiment 11d, wherein the mixture is subjected to the tandem mass spectrometry comprising ECD via direct infusion or flow-infusion.

Embodiment 12 is the method of any one embodiments 5-11e, wherein the reporter ion is a TMPP reporter ion.

Embodiment 12a is the method of embodiment 12, wherein the TMPP reporter ion has a nominal mass-to-charge (m/z) of about 533 Da, about 573 Da, or about 590 Da.

Embodiment 12b is the method of embodiment 12a, wherein the TMPP reporter ion has a nominal mass-to-charge (m/z) of about 533 Da.

Embodiment 12c is the method of embodiment 12b, wherein the TMPP reporter ion has an exact mass-to-charge (m/z) of about 533.1935.

Embodiment 12d is the method of embodiment 12b or 12c, wherein the labeled peptide is identified by detecting a second TMPP reporter ion in the ETD or ECD or other electron-induced dissociation mass spectrum of the TMPP labeled peptide, and the second TMPP reporter ion has a nominal mass-to-charge (m/z) of about 590 Da.

Embodiment 12e is the method of embodiment 12d, wherein the second TMPP reporter ion has an exact mass-to-charge (m/z) of 590.2150.

Embodiment 12f is the method of any one of embodiments 12a-12e, wherein the labeled peptide is identified by detecting a second or third TMPP reporter ion in the ETD or ECD or HCD or other electron-induced dissociation mass spectrum of the TMPP labeled peptide, and the second or third TMPP reporter ion has a nominal mass-to-charge (m/z) of about 573 Da.

Embodiment 12g is the method of embodiment 12f, wherein the second or third TMPP reporter ion has an exact mass-to-charge (m/z) of 573.1884.

Embodiment 12h is the method of any one of embodiments 12-12g, wherein the TMPP reporter ion is generated from a charge loss.

Embodiment 12i is the method of any one embodiments 12-12h, wherein the TMPP reporter ion is a predominant product ion in the ETD or ECD or other electron-induced dissociation mass spectrum.

Embodiment 12j is the method of any one of embodiments 5-12i, wherein the TMPP reporter ion is generated from a doubly charged peptide.

Embodiment 12k is the method of embodiment 12, wherein the TMPP reporter ion is TMPP⁺.

Embodiment 12l is the method of embodiment 12, wherein the TMPP reporter ion is TMPP-Ac-NH₂⁺.

Embodiment 12m is the method of embodiment 12, wherein the TMPP reporter ion is TMPP-Ac⁺.

Embodiment 13 is the method of any one of embodiments 5-12m, wherein the second mass spectrometry is collision-induced dissociation (CID) mass spectrometry resulting in CID tandem mass spectrometry (CID-MS²) and the second mass spectrum is a CID mass spectrum.

Embodiment 13a is the method of any one of embodiments 5-12m, wherein the second mass spectrometry is higher-energy collisional dissociation (HCD) mass spectrometry resulting in HCD tandem mass spectrometry (HCD-MS²) and the second mass spectrum is an HCD mass spectrum.

Embodiment 13b is the method of any one of embodiments 5-12m, wherein second mass spectrometry is ultraviolet photodissociation (UVPD) mass spectrometry resulting in UVPD tandem mass spectrometry (UVPD-MS²) and the second mass spectrum is an UVPD mass spectrum.

Embodiment 14 is the method of any one of embodiments 5-13b, wherein the TMPP reporter ion triggers the second mass spectrometry.

Embodiment 14a is the method of any one of embodiments 5-14, wherein the TMPP reporter ion triggers the second mass spectrometry via intensity and m/z.

Embodiment 14b is the method of embodiment 14 or 14a, wherein a filter (mass tolerance) is used to trigger the second mass spectrometry.

Embodiment 14c is the method of embodiment 14b, wherein the filter is set upon a base peak of precise mass at 533.1935 or 590.2150 Da with mass tolerance ranging between 1-20 ppm for triggering, such as a mass tolerance of 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, or any number between therefore, preferably 5 ppm.

Embodiment 14d is the method of embodiment 14 or 14a, wherein an intensity of the reporter ion is used as a threshold to trigger the second mass spectrometry.

Embodiment 14e is the method of embodiment 14d, wherein the intensity is set at 10% or more of the intensity of a base peak.

Embodiment 14f is the method of any one of embodiments 5-14e, wherein the TMPP reporter ion triggers the collision-induced dissociation (CID) mass spectrometry resulting in CID tandem mass spectrometry (CID-MS²).

Embodiment 14g is the method of any one of embodiments 5-14e, wherein the TMPP reporter ion triggers the higher-energy collisional dissociation (HCD) mass spectrometry resulting in HCD tandem mass spectrometry (HCD-MS²).

Embodiment 14h is the method of any one of embodiments 5-14e, wherein the TMPP reporter ion triggers the ultraviolet photodissociation (UVPD) mass spectrometry resulting in UVPD tandem mass spectrometry (UVPD-MS²).

Embodiment 15 is the method of any one of embodiments 5-14h, wherein the method is high-throughput.

Embodiment 16 is the method of any one of embodiments 1-15, the ETD or ECD or other electron-induced dissociation mass spectrum and the second mass spectrum are analyzed by comparing with information in a database or a spectral library.

Embodiment 16a is the method of embodiment 16, wherein the ETD mass spectrum and the second mass spectrum are analyzed.

Embodiment 16b is the method of embodiment 16, wherein the ECD mass spectrum and the second mass spectrum are analyzed.

Embodiment 16c is the method of any one of embodiments 16-16b, wherein the second mass spectrum is a CID mass spectrum.

Embodiment 16d is the method of any one embodiments 16-16b, wherein the second mass spectrum is an HCD mass spectrum.

Embodiment 16e is the method of any one embodiments 16-16b, wherein the second mass spectrum is a UVPD mass spectrum.

Embodiment 17 is the method of any one of embodiments 5-16e, wherein the method eliminates false-positive identification of the clipping site.

Embodiment 17a is the method of embodiment 17, wherein the false-positive identification is caused by an unlabeled polypeptide or peptide.

Embodiment 17b is the method of embodiment 17, wherein the false-positive identification is caused by labeling at a lysine residue.

Embodiment 17c is the method of embodiment 17, wherein the false-positive identification is caused by labeling at a tyrosine residue.

Embodiment 18 is a method of identifying a clipping site on a protein, the method comprising:
(i) obtaining a sample containing one or more clipped polypeptides of the protein;
(ii) labeling the one or more clipped polypeptides with a N-tris(2,4,6-trimethoxyphenyl)phosphonium acetyl (TMPP) to thereby obtain one or more TMPP labeled clipped polypeptides;
(iii) digesting the one or more TMPP labeled clipped polypeptides to generate a mixture comprising unlabeled peptides and TMPP labeled peptides;
(iv) optionally subjecting the mixture to liquid chromatography (LC) to generate elutes of the LC;
(v) subjecting the elutes from step iv) or the mixture from step iii) to electron transfer dissociation (ETD) tandem mass spectrometry to thereby generate an ETD or ECD or other electron-induced dissociation mass spectrum for each of the TMPP labeled peptides;
(vi) detecting or separating a TMPP reporter ion in the ETD or ECD or other electron-induced dissociation mass spectrum;
(vii) upon detection or separation of the TMPP reporter ion, subjecting each of the TMPP labeled peptides to collision-induced dissociation (CID) mass spectrometry or higher-energy collisional dissociation (HCD) mass spectrometry or ultraviolet photodissociation (UVPD) mass spectrometry to thereby generate a CID or HCD or UPVD mass spectrum for each of the TMPP labeled peptides, respectively; and
(viii) identifying the clipping site on the protein by analyzing the ETD or ECD or other electron-induced dissociation mass spectrum and the CID or HCD or UPVD mass spectrum.

Embodiment 19 is the method of embodiment 18, wherein the protein is an enzyme, an antibody (e.g., a monoclonal antibody, bi-specific antibody, tri-specific antibody, tetra-specific antibody) or antigen binding fragment thereof, a biomolecular antigen, a fusion protein, a fusion-peptide, a scaffold protein or peptide, a protein or peptide drug conjugate, or any other polypeptide or peptide useful as a therapeutic or diagnostic modality.

Embodiment 19a is the method of embodiment 19, wherein the protein is a therapeutic protein.

Embodiment 19b is the method of embodiment 19, wherein the protein is a non-therapeutic protein.

Embodiment 19c is the method of any one of embodiments 18-19b, wherein the clipped polypeptide of the protein has one or more clipped sites.

Embodiment 19d is the method of any one of embodiments 18-19c, wherein the ETD is used in the method.

Embodiment 19e is the method of any one of embodiments 18-19d, wherein the ECD is used in the method.

Embodiment 19f is the method of any one of embodiments 18-19e, wherein the efficiency of the TMPP labeling is 1%, 10%, 20%, 30%, 40%, 50%, 60%, 70%, 80%, 90%, 100%, or any number between thereof.

Embodiment 20 is the method of any one of embodiments 18-19f, wherein the LC is high performance liquid chromatogram (HPLC) or ultra-performance liquid chromatogram (UPLC), preferably HPLC.

Embodiment 20a is the method of embodiment 20, wherein the TMPP labeled peptide elutes later in a reverse phase gradient compared to the corresponding unlabeled peptide.

Embodiment 20b is the method of any one of embodiments 18-19b, wherein the mixture from step iii) is subjected to tandem mass spectrometry comprising electron transfer dissociation (ETD).

Embodiment 20c is the method of embodiment 20b, wherein the mixture is subjected to the tandem mass spectrometry comprising ETD via direct infusion or flow-infusion.

Embodiment 20d is the method of any one of embodiments 18-19a, wherein the mixture from step iii) is subjected to tandem mass spectrometry comprising electron capture dissociation (ECD).

Embodiment 20e is the method of embodiment 20c, wherein the mixture is subjected to the tandem mass spectrometry comprising ECD via direct infusion or flow-infusion.

Embodiment 21 is the method of any one of embodiments 18-20c, wherein the TMPP reporter ion has a nominal mass-to-charge (m/z) of about 533 Da, about 573 Da, or about 590 Da.

Embodiment 21a is the method of embodiment 21, wherein the TMPP reporter ion has a nominal mass-to-charge (m/z) of about 533 Da.

Embodiment 21b is the method of embodiment 21a, wherein the TMPP reporter ion has an exact mass-to-charge (m/z) of 533.1935.

Embodiment 21c is the method of embodiment 21a or 21b, further comprising detecting a second TMPP reporter ion in the ETD or ECD or other electron-induced dissociation mass spectrum, and the second TMPP reporter ion has a nominal mass-to-charge (m/z) of about 590 Da.

Embodiment 21d is the method of embodiment 21c, wherein the second TMPP reporter ion has an exact mass-to-charge (m/z) of 590.2150.

Embodiment 21e is the method of any one of embodiments 21a-21d, wherein the TMPP reporter ion has a nominal mass-to-charge (m/z) of about 573 Da.

Embodiment 21f is the method of embodiment 21e, wherein the TMPP reporter ion has an exact mass-to-charge (m/z) of 573.1884.

Embodiment 21g is the method of any one of embodiments 21-21f, wherein the TMPP reporter ion is generated from a charge loss.

Embodiment 21h is the method of any one of embodiments 21-21g, wherein the TMPP reporter ion is a predominant product ion in the ETD or ECD or other electron-induced dissociation mass spectrum.

Embodiment 21i is the method of any one of embodiments 21-21h, wherein the TMPP reporter ion is generated from a doubly charged peptide.

Embodiment 21j is the method of embodiment 21, wherein the TMPP reporter ion is TMPP⁺.

Embodiment 21k is the method of embodiment 21, wherein the TMPP reporter ion is TMPP-Ac-NH₂+.

Embodiment 21l is the method of embodiment 21, wherein the TMPP reporter ion is TMPP-Ac⁺.

Embodiment 22 is the method of any one of embodiments 18-21l, wherein the TMPP reporter ion triggers the collision-induced dissociation (CID) mass spectrometry resulting in CID tandem mass spectrometry (CID-MS²), or the higher-energy collisional dissociation (HCD) mass spectrometry resulting in HCD tandem mass spectrometry (HCD-MS²), or ultraviolet photodissociation (UVPD) mass spectrometry resulting in UVPD tandem mass spectrometry (UVPD-MS²).

Embodiment 22a is the method of embodiment 22, wherein the TMPP reporter ion triggers the CID mass spectrometry resulting in CID-MS² or HCD mass spectrometry resulting in HCD-MS² or UVPD mass spectrometry resulting in UVPD-MS² via intensity and m/z.

Embodiment 22b is the method of embodiment 22 or 22a, wherein a filter (mass tolerance) is used to trigger the second mass spectrometry.

Embodiment 22c is the method of embodiment 22b, wherein the filter is set upon a base peak of precise mass at 533.1935 or 590.2150 Da with mass tolerance ranging between 1-20 ppm for triggering, such as a mass tolerance of 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, or any number between therefore, preferably 5 ppm.

Embodiment 22d is the method of embodiment 22 or 22a, wherein an intensity of the reporter ion is used as a threshold to trigger the second mass spectrometry.

Embodiment 22e is the method of embodiment 22d, wherein the intensity is set at 10% or more of the intensity of a base peak.

Embodiment 23 is the method of any one of embodiments 18-22e, wherein the method is high-throughput.

Embodiment 24 is the method of any one of embodiments 18-23, the ETD or ECD or other electron-induced dissociation mass spectrum and the CID mass spectrum are analyzed by comparing with information in a database or a spectral library.

Embodiment 24a is the method of any one of embodiments 18-23, the ETD or ECD or other electron-induced dissociation mass spectrum and the HCD mass spectrum are analyzed by comparing with information in a database or a spectral library.

Embodiment 25 is the method of any one of embodiments 18-24a, wherein the method eliminates false-positive identification of the clipping site.

Embodiment 25a is the method of embodiment 25, wherein the false-positive identification is caused by an unlabeled polypeptide or peptide.

Embodiment 25b is the method of embodiment 25, wherein the false-positive identification is caused by labeling at a lysine residue.

Embodiment 25c is the method of embodiment 25, wherein the false-positive identification is caused by labeling at a tyrosine residue.

Embodiment 26 is a systems for identifying a clipping site on a polypeptide or characterizing a polypeptide in a sample.

Embodiment 26a is the system of embodiment 26, wherein the system comprises a liquid chromatography (LC) device and a tandem mass spectrometer.

Embodiment 26b is the system of embodiment 26 or 26a, wherein the tandem mass spectrometer comprises:
(i) a first ionization device;
(ii) a first mass to charge ratio filter or mass to charge ratio mass analyzer arranged and adapted in a first mode of operation to transmit ions having a mass to charge ratio within a first range;
(iii) a first ion mobility spectrometer, detector, or separator;
(iv) attenuation means for attenuating ions in a mode of operation;
(v) a control device configured to control the operation of the attenuation means so that ions having mass to charge ratios within the first range but having one or more undesired first charge states are substantially attenuated;
(vi) a second ionization device;
(vii) a second ion mobility spectrometer, detector, or separator; and
(viii) a data system configured to acquire non-mixed signals of fragment ions and to non-redundantly encode triggering ions, the non-redundant encoding being arranged to avoid or minimize repetitive overlapping of any two ion signals from different parent species at multiple repetitions of any individual gate time.

Embodiment 27 is the system of any one of embodiments 26-26b, wherein the LC device is a high performance liquid chromatography (HPLC) device.

Embodiment 28 is the system of any one of embodiments 26-27, wherein the sample is subject to the LC device to generate elutes.

Embodiment 28a is the system of embodiment 28, wherein the elutes are subjected to the tandem mass spectrometry to obtain a first mass spectrum and a second mass spectrum.

Embodiment 29 is the system of any one of embodiments 26-28, wherein the clipping site on the polypeptide or the polypeptide is labeled with a N-terminal labeling reagent.

Embodiment 29a is the system of embodiment 29, wherein the N-terminal labeling reagent is N-tris(2,4,6-trimethoxyphenyl)phosphonium acetyl (TMPP).

Embodiment 29b is the system of embodiment 29a, wherein the first ionization device generates a TMPP reporter ion.

Embodiment 29c is the system of embodiment 29b, wherein the TMPP reporter ion has a nominal mass-to-charge (m/z) of about 533 Da, about 573 Da, or about 590 Da.

Embodiment 29d is the system of any one of embodiments 29a-29c, wherein the TMPP reporter ion triggers the second mass spectrometry.

Embodiment 30 is the system of any one of embodiments 26-29, wherein the first ionization device is an electron-induced dissociation device.

Embodiment 30a is the system of embodiment 30, wherein the electron-induced dissociation device is an electron transfer dissociation (ETD) device or electron capture dissociation (ECD) device.

Embodiment 30b is the system of embodiment 30a, wherein the first mass spectrum is an ETD or ECD mass spectrum.

Embodiment 31 is the system of any one of embodiments 26-30, wherein the second ionization device is a collision-induced dissociation (CID) device, higher-energy collisional dissociation (HCD) device, or ultraviolet photodissociation (UVPD) device.

Embodiment 31a is the system of embodiment 31, wherein the second mass spectrum comprises a CID, HCD, or UVPD mass spectrum.

Embodiment 32 is the system of embodiment 30b or 31a, wherein the first mass spectrum and the second mass spectrum are analyzed by comparing with information in a database or a spectral library.

Embodiment 33 is the system of any one of embodiments 26-32, wherein the mass spectrometer further comprises a collision device, fragmentation device, or reaction device.

Embodiment 34 is the system of any one of embodiments 26-33, wherein the attenuation means comprises an ion gate or ion barrier.

Embodiment 34a is the system of any one of embodiments 26-34, wherein the attenuation means is arranged downstream of the ion mobility spectrometer or separator.

Embodiment 35 is the system of any one of embodiments 26-34a, wherein the first mass to charge ratio filter or mass to charge ratio mass analyzer is arranged and adapted in the first mode of operation to attenuate ions having mass to charge ratios outside of the first range.

Embodiment 35a is the system of any one of embodiments 26-35, wherein the first mass to charge ratio filter or mass to charge ratio mass analyzer is arranged upstream or downstream of said ion mobility spectrometer or separator.

Embodiment 36 is the system of any one of embodiments 26-35a, wherein the first undesired charge state is selected from one or more of the following: (i) singly charged; (ii) doubly charged; (iii) triply charged; (iv) quadruply charged; (v) quintuply; and (vi) multiply charged.

Embodiment 37 is the system of any one of embodiments 26-36, wherein the mass spectrometer further comprises an ion guide, ion trap or ion trapping region arranged upstream of said ion mobility spectrometer or separator, wherein said ion guide, ion trap or ion trapping region is arranged to trap, store or accumulate ions and then to periodically pulse ions into or towards said ion mobility spectrometer or separator.

Embodiment 38 is a reporter ion for identifying a clipping site on a polypeptide and/or a reporter ion for characterizing a polypeptide.

Embodiment 38a is the reporter ion of embodiment 38, wherein the clipping site is labeled with an N-terminal labeling reagent.

Embodiment 38b is the reporter ion of embodiment 38, wherein the polypeptide is labeled with an N-terminal labeling reagent.

Embodiment 38c is the reporter ion of embodiment 38a or 38b, wherein the N-terminal labeling reagent is ionized to generate the reporter ion.

Embodiment 38d is the reporter ion of embodiment 38a or 38b, wherein the N-terminal labeling reagent is N-tris(2,4,6-trimethoxyphenyl)phosphonium acetyl (TMPP).

Embodiment 38e is the reporter ion of embodiment 38d, wherein the TMPP is ionized to generate the reporter ion.

Embodiment 39 is the reporter ion of any one of embodiments 38a-38c, wherein the N-terminal labeling reagent is ionized by a mass spectrometer to generate the reporter ion.

Embodiment 39a is the reporter ion of any one of embodiments 38a-38c, wherein the N-terminal labeling reagent is ionized by a mass spectrometry technique to generate the reporter ion.

Embodiment 40 is the reporter ion of embodiment 38d or 38e, wherein the TMPP is ionized by a mass spectrometer to generate the reporter ion.

Embodiment 40a is the reporter ion of embodiment 38d or 38e, wherein the TMPP is ionized by a mass spectrometry technique to generate the reporter ion.

Embodiment 41 is the reporter ion of embodiment 39 or 40, wherein the mass spectrometer is a tandem mass spectrometer.

Embodiment 41a is the reporter ion of embodiment 41, wherein the tandem mass spectrometer comprises an electron transfer dissociation (ETD) device, electron capture dissociation (ECD) device, collision-induced dissociation (CID) device, higher-energy collisional dissociation (HCD) device, ultraviolet photodissociation (UVPD) device, or any combination thereof.

Embodiment 42 is the reporter ion of embodiment 39a or 40a, wherein the mass spectrometry technique is a tandem mass spectrometry technique.

Embodiment 42a is the reporter ion of embodiment 42, wherein the tandem mass spectrometry technique is any tandem mass spectrometry technique described herein. For example, in some embodiments, the tandem mass spectrometry technique comprises an electron transfer dissociation (ETD), electron capture dissociation (ECD), collision-induced dissociation (CID), higher-energy collisional dissociation (HCD), ultraviolet photodissociation (UVPD), or any combination thereof.

Embodiment 42b is the reporter ion of embodiment 42a, wherein the reporter ion has a nominal mass-to-charge (m/z) of about 533 Da, about 573 Da, or about 590 Da.

Embodiment 42c is the reporter ion of embodiment 42a, wherein the reporter ion is a compound having a structure of:

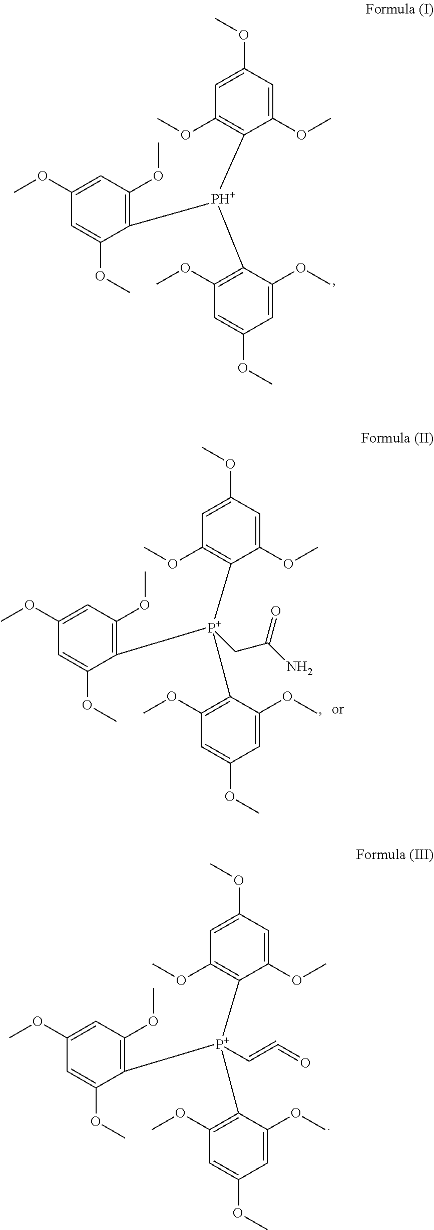

Embodiment 43 is a composition for identifying a clipping site on a polypeptide, wherein the composition comprises at least one reporter ion described herein and a polypeptide.

Embodiment 44 is a composition for characterizing a polypeptide, wherein the composition comprises at least one reporter ion described herein and a polypeptide.

Embodiment 45 is a kit for identifying a clipping site on a polypeptide in a sample, wherein the kit comprises: a reporter ion for identifying a clipping site on a polypeptide.

Embodiment 45a is the kit of embodiment 45, wherein the clipping site is labeled with N-tris(2,4,6-trimethoxyphenyl)phosphonium acetyl (TMPP).

Embodiment 45b is the kit of embodiment 45a, wherein the TMPP is ionized to generate the reporter ion.

Embodiment 46 is a kit for characterizing a polypeptide in a sample, wherein the kit comprises: a reporter ion for characterizing a polypeptide.

Embodiment 46a is the kit of embodiment 46, wherein the polypeptide is labeled with N-tris(2,4,6-trimethoxyphenyl)phosphonium acetyl (TMPP).

Embodiment 46b is the kit of embodiment 46a, wherein the TMPP is ionized to generate the reporter ion.

The following examples are to further illustrate the nature of the invention. It should be understood that the following examples do not limit the invention and that the scope of the invention is determined by the appended claims.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Detection of Diagnostic Ions by TMPP Labeling of N-Termini and Electron Transfer Dissociation (ETD)

The diagnostic utility of the TMPP reporter ions by labeling the NIST antibody standard with TMPP and tryptic peptide mapping by data dependent ETD-MS/MS was investigated. The NIST antibody had mature N-termini from the light and heavy chains. One of the two surrogate peptides corresponded to the N-termini of the NIST antibody light chain that had a free primary amine while the N-terminal NIST antibody of the heavy chain consisted of a secondary amine due the cyclization of glutamine to form pyroglutamic acid residue of the N-terminus. Any neo N-termini on the NIST antibody was a potential degradation or clipping product of the molecule during storage.

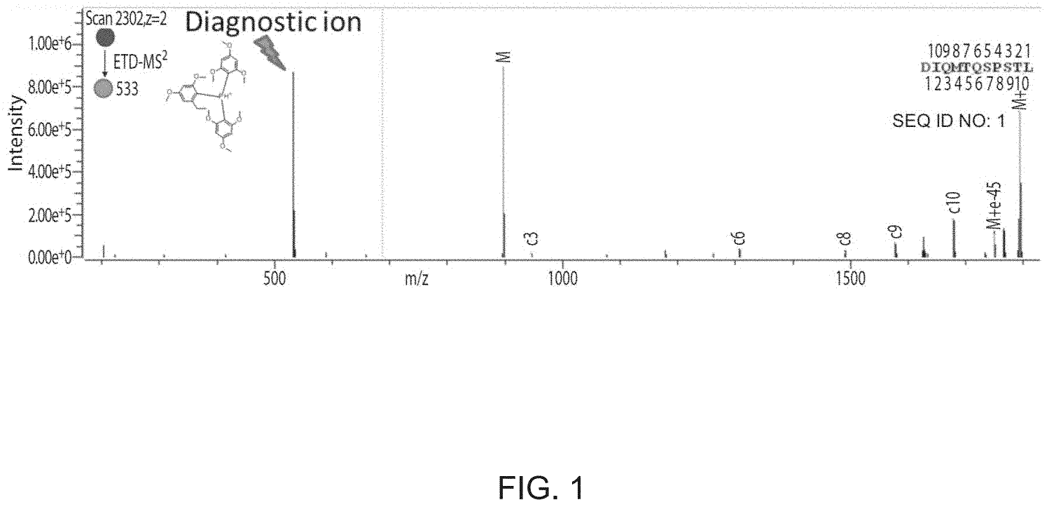

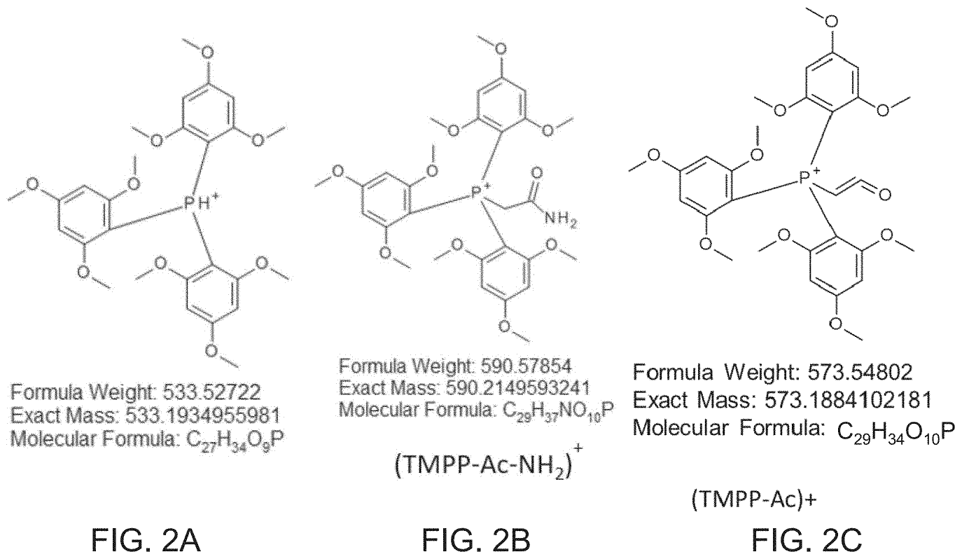

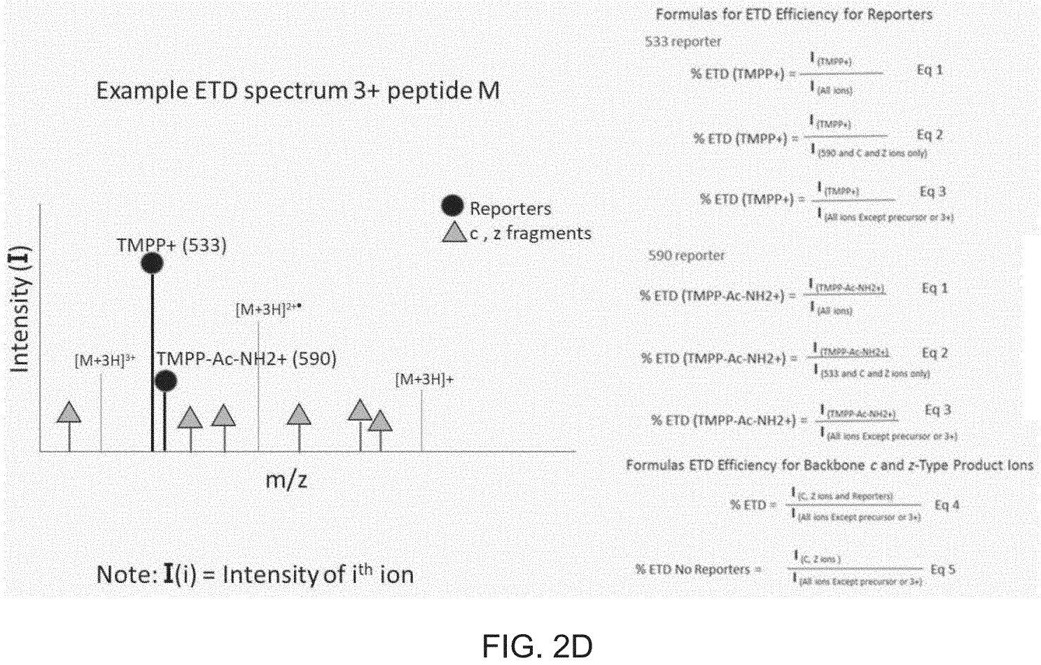

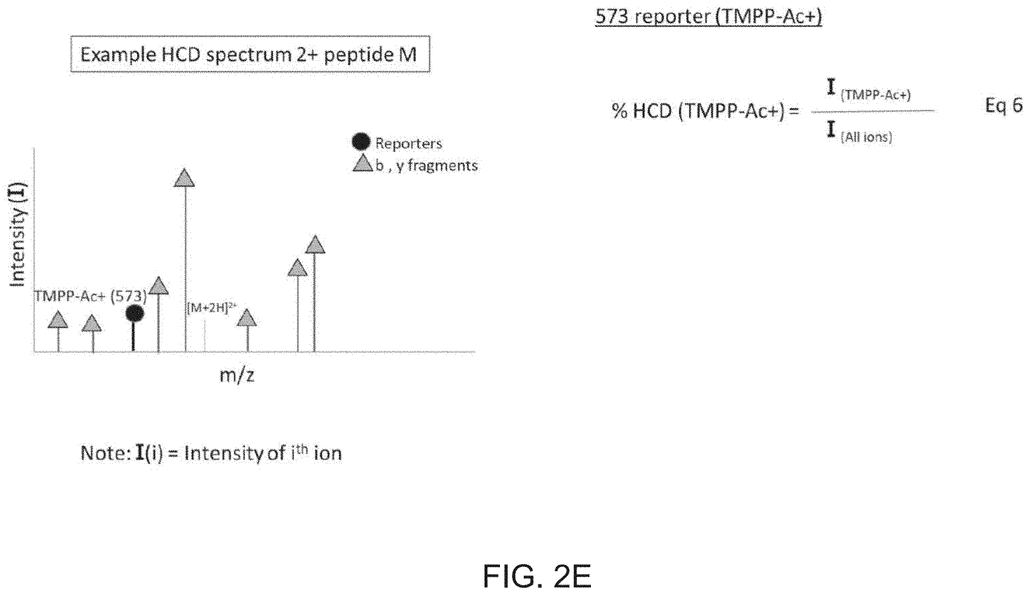

FIG. 1 shows the ETD product ion spectrum of the peptide corresponding to light chain N-terminal sequence of the NIST antibody. The mass spectrum consisted predominantly of the diagnostic TMPP reporter ion (m/z=533 Da) and of c-type backbone product ions that consisted of the N-terminal TMPP tag. It was interesting to note that this peptide sequence was generated due to in-source fragmentation of a larger tryptic peptide that produced the reporter ion to a much lesser degree during ETD, presumably due to charge sequestration on the C-terminal arginine residue (Xia et al., Journal of the American Chemical Society, 2007, 129:12232-12243). Nevertheless, the approach that was taken included electron transfer dissociation to produce a dominant reporter ion peak, localize the TMPP tag on the N-terminus using high-sequence coverage, and reporter ion triggering of a complementary activation event to confirm the presence of a mature N-terminus of the NIST light chain. The absence of additional low-level of clipped species due to degradation of the NIST antibody was also confirmed by examining and filtering ETD-MS/MS spectra that has 533 and 590 diagnostic reporter ions in the entire data set. See FIGS. 2A-B for the structure of reporter ions at 533 and 590 Da.

The triggered CID approach made the filtering of the data amenable to manual inspection due to the low occurrences of triggered MS2 scans that confirmed the presence of reporter ions generated from ETD-MS/MS events for the entire data set. This eliminated the need for in-slico approaches or manual inspection of ETD-MS/MS scans that have reporter ion peaks.

### Example 2: Rapid Separation of TMPP Labeled Peptides and Retention Time Predictability

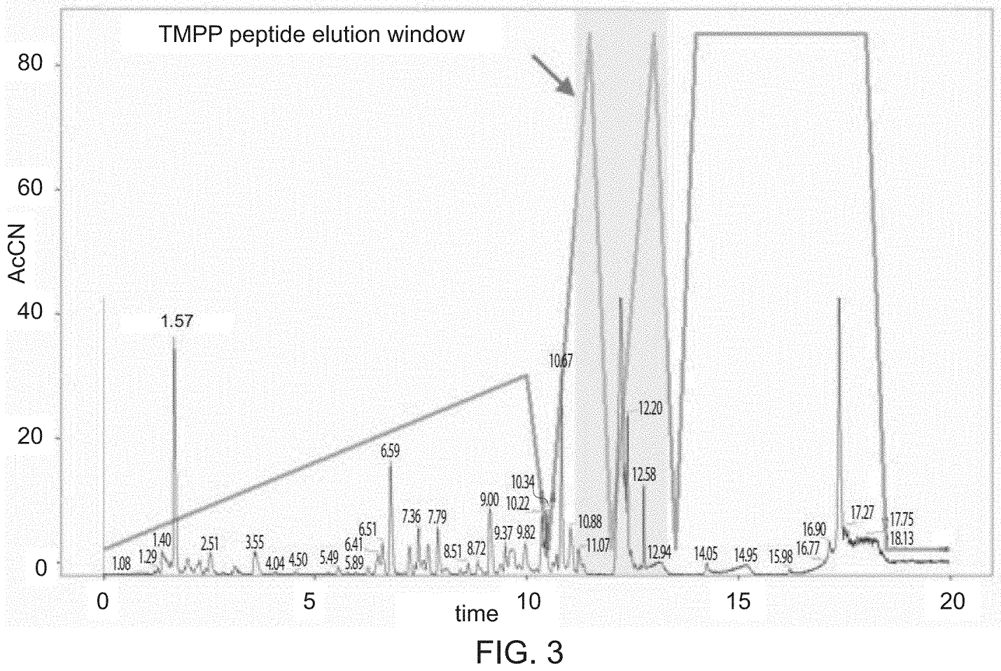

The rapid separation conditions for high-throughput detection of TMPP labeled peptides were evaluated next. Complete reverse phase separation of peptides, clean-up, and re-equilibration were achieved with a total run time of 20 minutes. FIG. 3 shows the reverse-phase chromatographic buffer gradient and the corresponding total ion chromatogram of NIST digest after TMPP labeling. It was important to note that most unlabeled peptides eluted at 2˜30% organic in a 10 minutes long shallow gradient. The surrogate peptides corresponded to the N-termini of the NIST antibody light chain eluted at 12 min. TMPP labeling increased the hydrophobicity of the N-terminal peptides and it was conceivable that TMPP labeled peptides were mostly observed during the two, sharp gradient with rapid ramps: 2-85% of organic solvent in 1 minute. The ability to separate and improve the retention time predictability of the TMPP labeled peptides further improved the specificity and reduced false-positive identification of peptides.

It was also important to note that the corresponding unlabeled NIST mAb N-terminus peptide counterpart at 3.5 min was also observed. It was conceivable that complex samples, having multiple clipped species, consist of both TMPP labeled and unlabeled peptide counterparts (due to labeling being less than 100% efficient), these unlabeled peptides eluted earlier in the gradient and were usually less in intensity and in some instances below the LOD compared to a labeled peptide.

The likelihood of observing unlabeled peptides was then assessed, and the retention time predictability of TMPP labeled peptides was evaluated by derivatizing 15 synthetic peptides standards from the NIST sequence. The 20 min long HPLC gradient injection of these peptide mixtures was monitored by recording the retention time and intensities of unlabeled and TMPP labeled peptides. The reaction efficiency for TMPP labeling was assessed by obtaining a peak area ratio of the TMPP labeled peptide normalized to the total intensity observed for that peptide. In the analysis, 15 TMPP peptides eluted between 12-14 min with labeling efficiency for these peptides range from 8-100% with 8 of the 15 peptides reacting with 100% efficiency. It was noteworthy that all TMPP labeled peptides were identified by MS/MS despite varying reaction efficiencies. The unlabeled peptide counterparts eluted at a much wider retention time windows of 4-12 min. Due to the high reaction efficiency, only 7 of 15 unlabeled peptides were identified by MS/MS while the remaining 8 unlabeled peptides were detected at MS1 yet did not trigger MS2-ETD due to low LOD. The labeling of peptides enabled improved identification and retention time predictability. The LC run times of the overall gradient can be shortened for rapid identification of clip-sites for less complex samples or extended for more complex mixtures.

### Example 3: Diagnostic Ions of TMPP Labeled Synthetic Peptide Standards

Several synthetic peptide standards corresponding to NIST mAb were derivatized with TMPP labels and subjected to LC-MS using ETD-MS² and diagnostic reporter ion triggered-MS² CID events. The propensity to generate diagnostic TMPP reporter ions (m/z=533 Da, and 590 Da) in the ETD spectra were then evaluated using these synthetic peptide standards that had different lengths, amino acid compositions, and sites for TMPP labeling. Table 1 shows that most peptides triggered a subsequent MS2-CID scan upon detecting a TMPP reporter ion. The triggered scans showed a dependency on the reporter ion intensity, i.e., TMPP⁺533 m/z. Several efficiency estimates of the ETD spectra of NIST peptides have been derived for TMPP⁺ and TMPP-Ac⁺ reporter ions for TMPP derivatized peptides based on % efficiency calculations reported previously for fragmentation of peptide backbone bonds (Gunawardena et al., Journal of the American Chemical Society, 2005, 127:12627-12639). See FIG. 2D for the calculation of ETD efficiency estimates for these two reporter ions.

From synthetic NIST peptides, it was observed that TMPP⁺ efficiencies were significantly larger than the TMPP-Ac⁺ due to the reporter ion abundance differences. It was interesting to also note that the reporter ion intensity contributed significantly to the overall ETD efficiency of the backbone bonds, especially from the contribution of TMPP⁺ reporter ions.

TABLE 1
ETD efficiency of TMPP labeled NIST synthetic peptides.

| | | | | | | | % ETD | % | |
|---|---|---|---|---|---|---|---|---|---|
| | Sequence | Y | K | | triggered | % ETD | (TMPP- | ETD | % Efficiencies |
| Sequences | Length | Counts | Counts | Charges | MS2 | (TMPP+) | Ac+) | Efficiencies | No Reporters |
| tmpp-HK | 2 | 0 | 1 | 3 | yes | 29.1 | 4.4 | 93.5 | 62.2 |
| tmpp-CK | 2 | 0 | 1 | 2 | yes | 54.4 | 0.0 | 100.0 | 45.6 |
| tmpp-HK | 2 | 0 | 1 | 2 | yes | 53.9 | 1.1 | 73.4 | 33.1 |
| tmpp-EAK | 3 | 0 | 1 | 2 | yes | 46.2 | 11.4 | 93.8 | 39.8 |
| tmpp-EYK | 3 | 1 | 1 | 2 | yes | 46.1 | 17.6 | 94.0 | 34.1 |
| tmpp- | 4 | 0 | 1 | 2 | yes | 52.5 | 0.0 | 84.1 | 31.6 |
| GQPR | | | | | | | | | |
| (SEQ ID | | | | | | | | | |
| NO: 21) | | | | | | | | | |
| tmpp- | 4 | 0 | 0 | 2 | yes | 55.0 | 0.5 | 80.4 | 35.8 |
| TKPR | | | | | | | | | |
| (SEQ ID | | | | | | | | | |
| NO: 18) | | | | | | | | | |
| tmpp- | 5 | 0 | 0 | 2 | yes | 59.3 | 1.2 | 92.4 | 36.5 |
| SFNIR | | | | | | | | | |
| (TMPP- | | | | | | | | | |
| SEQ ID | | | | | | | | | |
| NO: 31) | | | | | | | | | |
| tmpp- | 4 | 0 | 1 | 2 | yes | 60.5 | 1.8 | 92.6 | 34.9 |
| VQWK | | | | | | | | | |
| (SEQ ID | | | | | | | | | |
| NO: 15) | | | | | | | | | |
| tmpp- | 17 | 1 | 1 | 4 | yes | 35.9 | 0.0 | 100.0 | 64.1 |
| VYACEVT | | | | | | | | | |
| HQGLSS | | | | | | | | | |
| PVTK | | | | | | | | | |
| (SEQ ID | | | | | | | | | |
| NO: 13) | | | | | | | | | |
| tmpp- | 5 | 1 | 1 | 2 | yes | 51.2 | 3.0 | 92.3 | 42.3 |
| ADYEK | | | | | | | | | |
| (SEQ ID | | | | | | | | | |
| NO: 12) | | | | | | | | | |
| tmpp- | 7 | 0 | 0 | 2 | yes | 69.7 | 0.9 | 72.3 | 21.2 |
| DTLMISR | | | | | | | | | |
| (SEQ ID | | | | | | | | | |
| NO: 16) | | | | | | | | | |
| tmpp- | 14 | 1 | 1 | 3 | yes | 0.3 | 0.0 | 100.0 | 99.7 |
| FNWYVD | | | | | | | | | |
| GVEVHN | | | | | | | | | |
| AK | | | | | | | | | |
| (SEQ ID | | | | | | | | | |
| NO: 17) | | | | | | | | | |
| tmpp- | 16 | 0 | 1 | 3 | No | 0.3 | 0.0 | 89.0 | 88.7 |
| WSVLTV | | | | | | | | | |
| LHQDWL | | | | | | | | | |
| NGK | | | | | | | | | |
| (SEQ ID | | | | | | | | | |
| NO: 20) | | | | | | | | | |
| tmpp- | 9 | 2 | 0 | 2 | yes | 52.4 | 0.4 | 66.7 | 31.5 |
| EEQYNS | | | | | | | | | |
| TYR (SEQ | | | | | | | | | |
| ID NO: 19) | | | | | | | | | |
| tmpp- | 11 | 1 | 0 | 2 | yes | 6.0 | 0.0 | 66.6 | 62.6 |
| EPQVYTL | | | | | | | | | |
| PPSR | | | | | | | | | |
| (SEQ ID | | | | | | | | | |
| NO: 22) | | | | | | | | | |

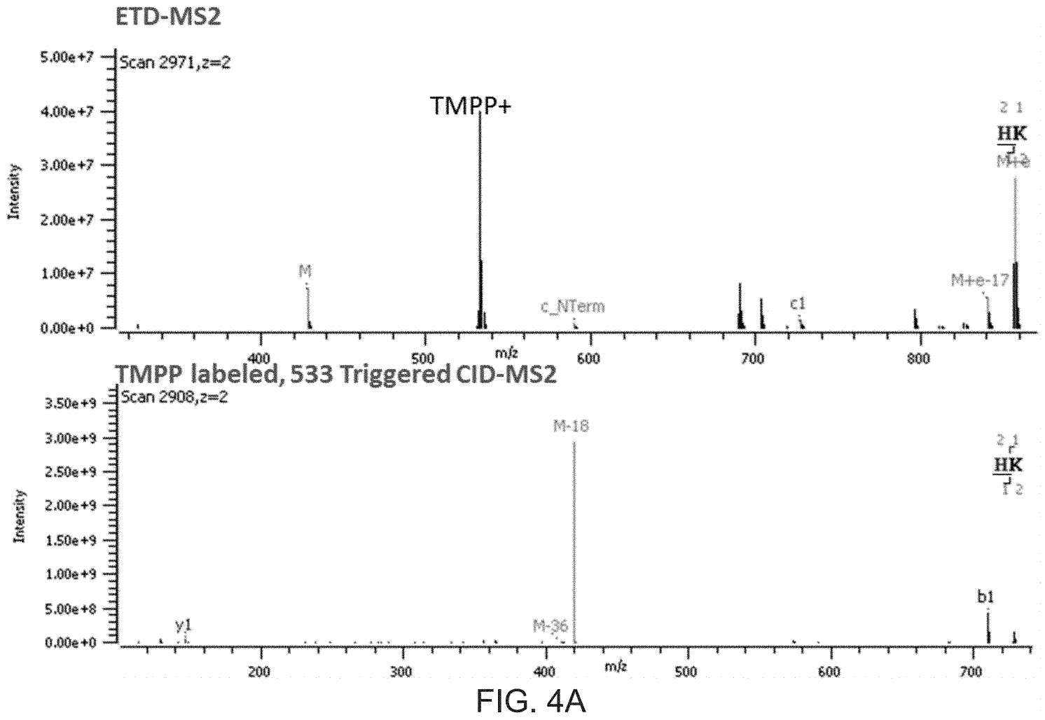

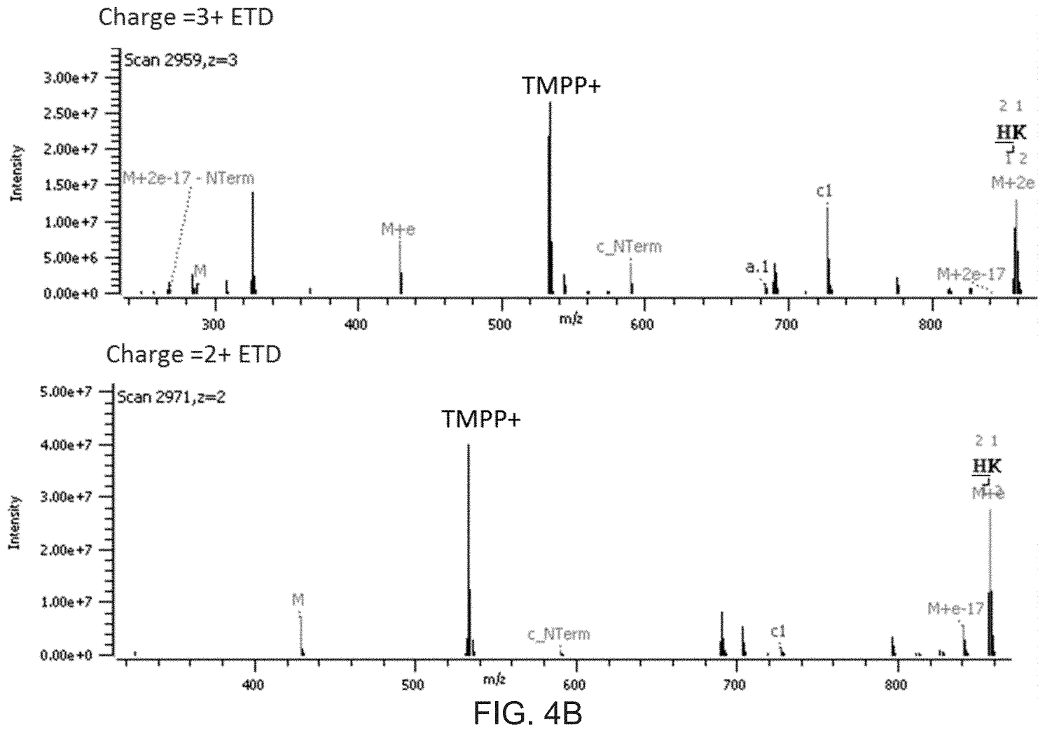

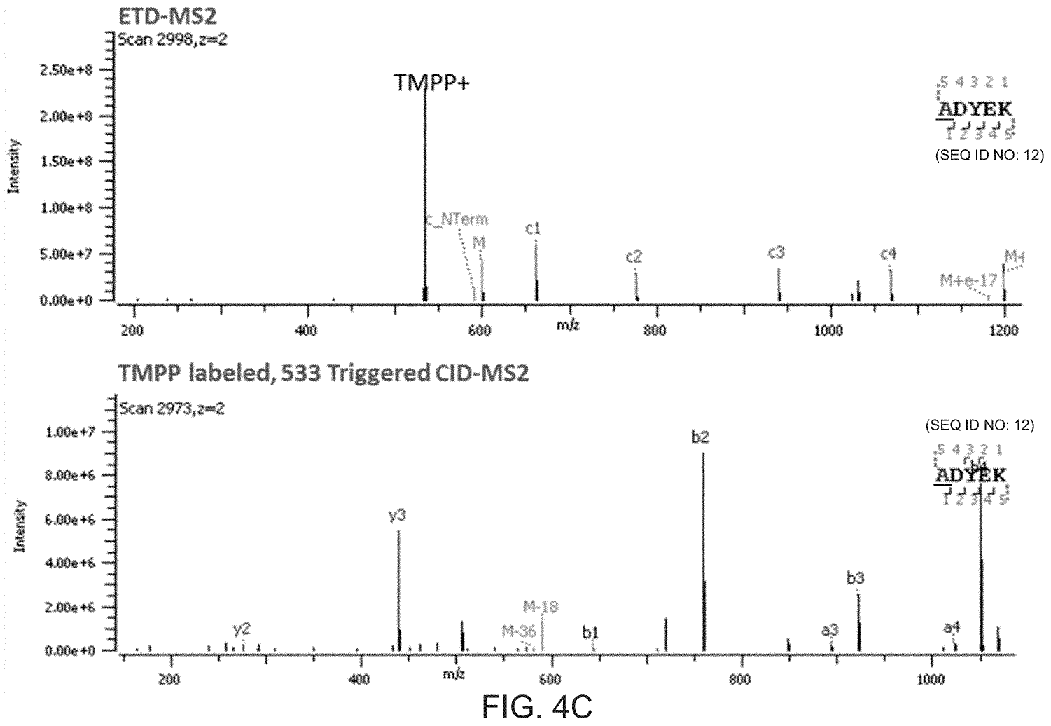

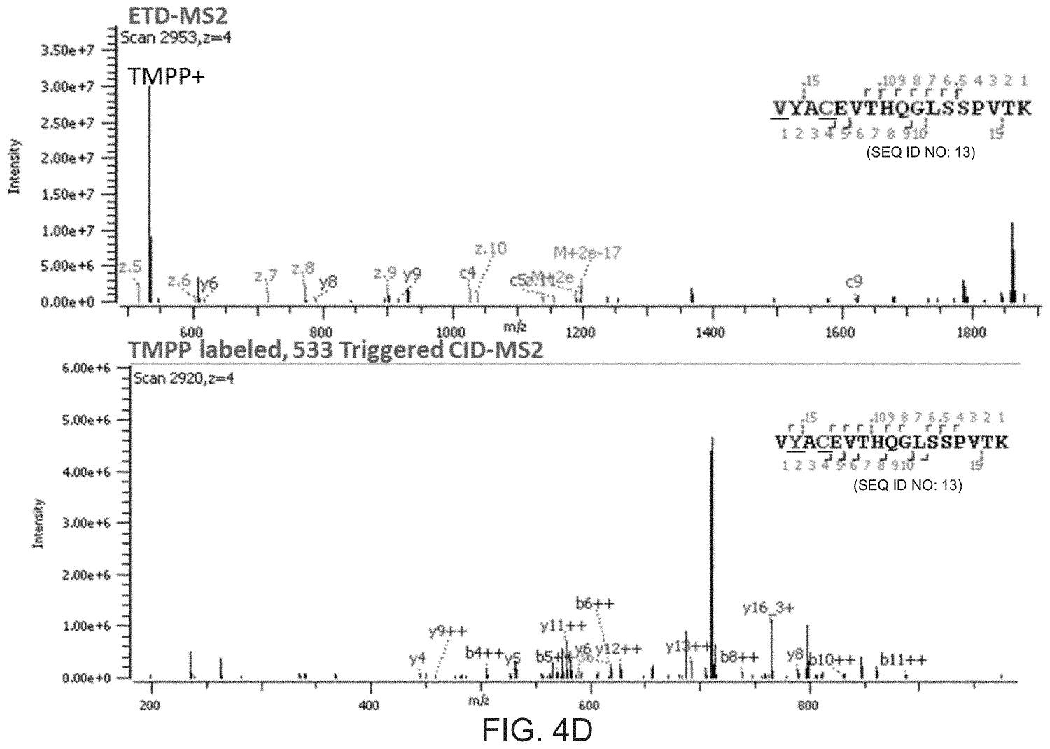

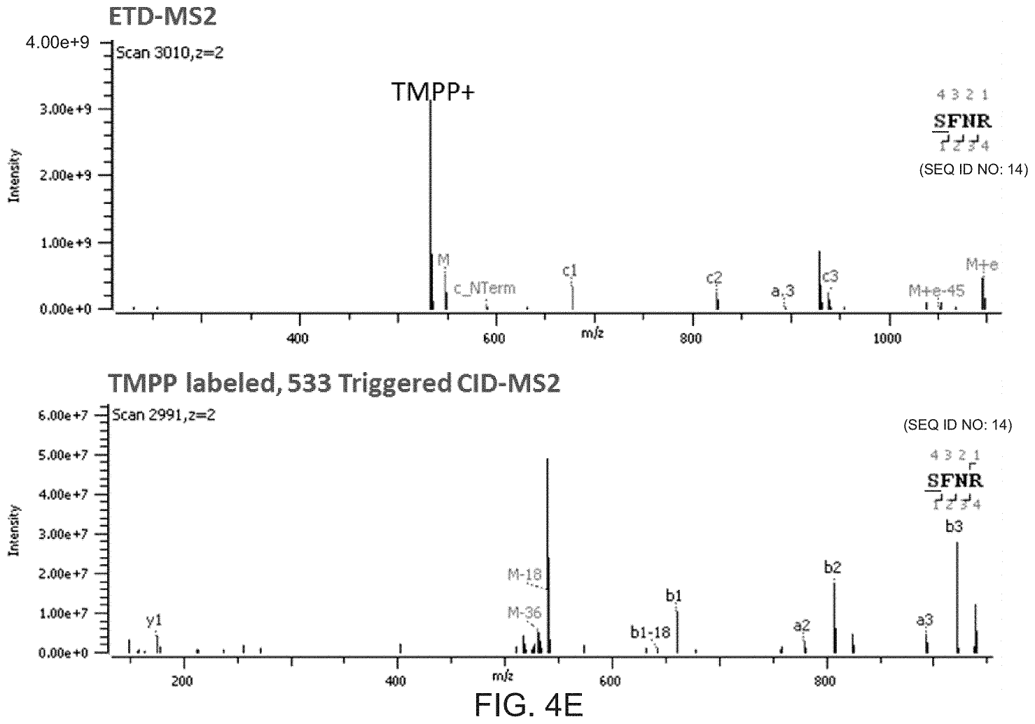

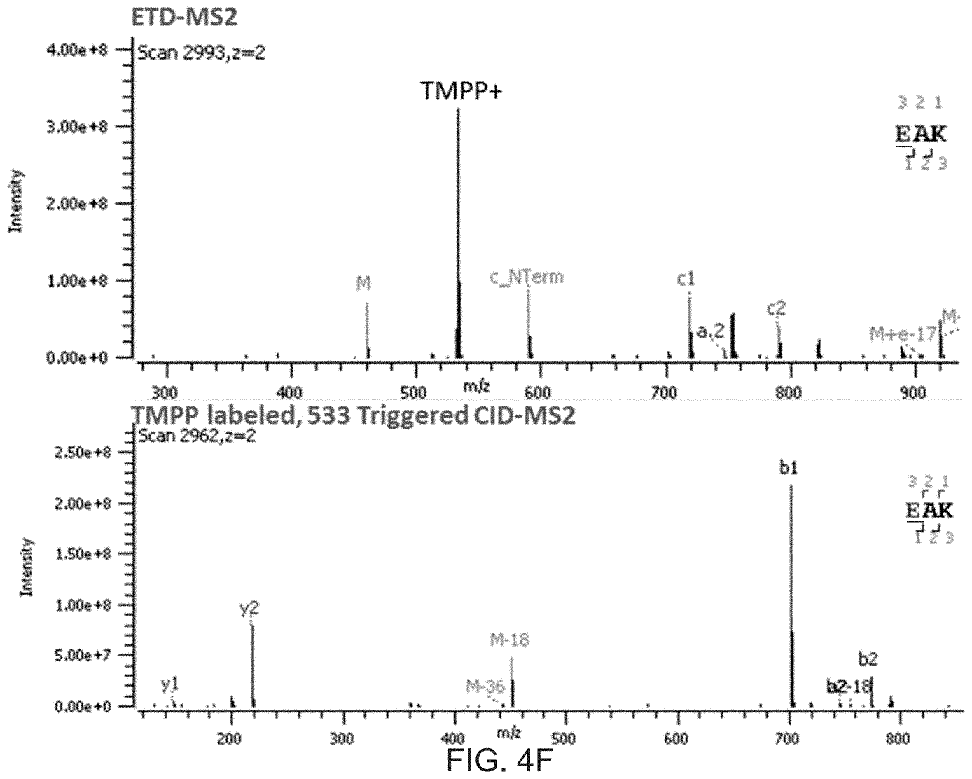

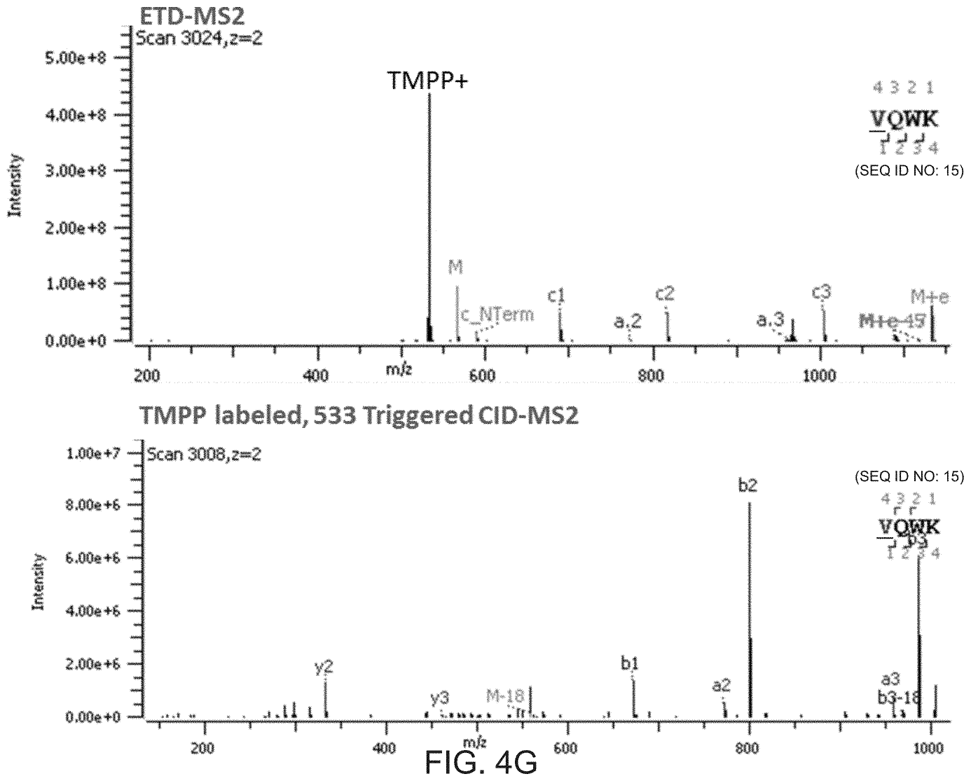

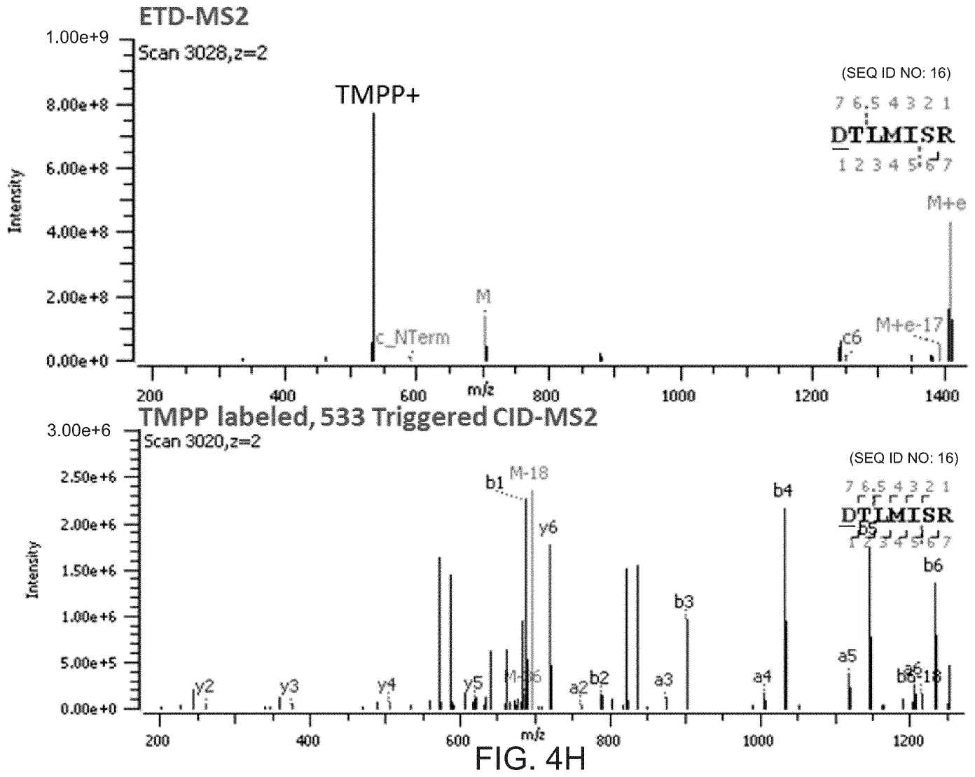

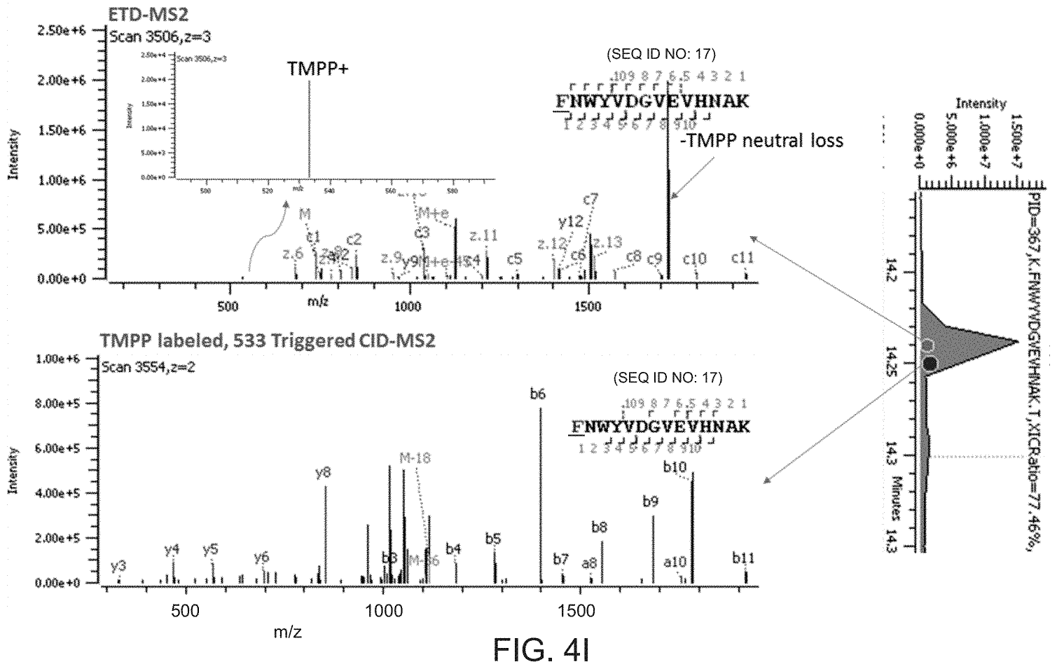

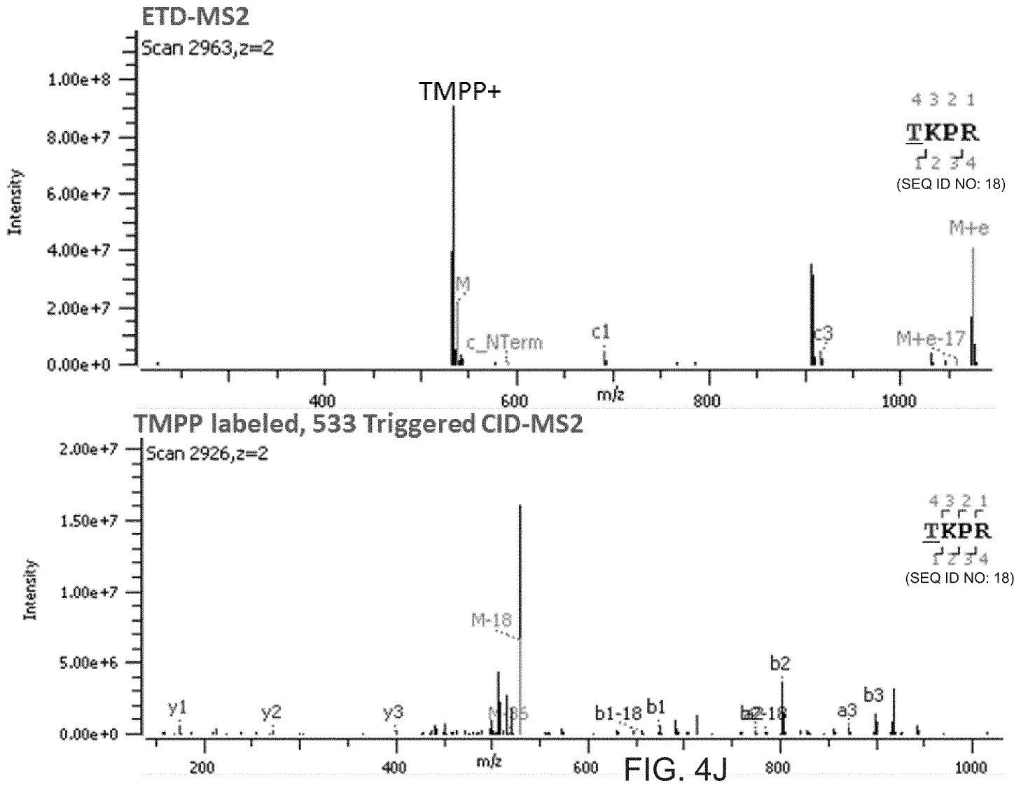

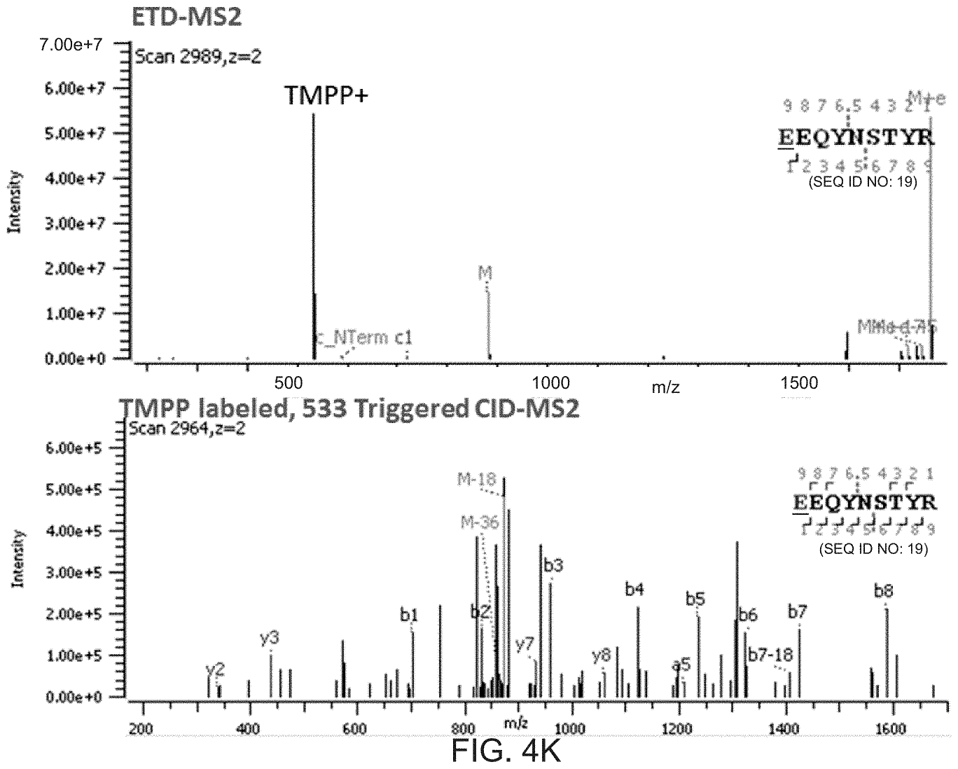

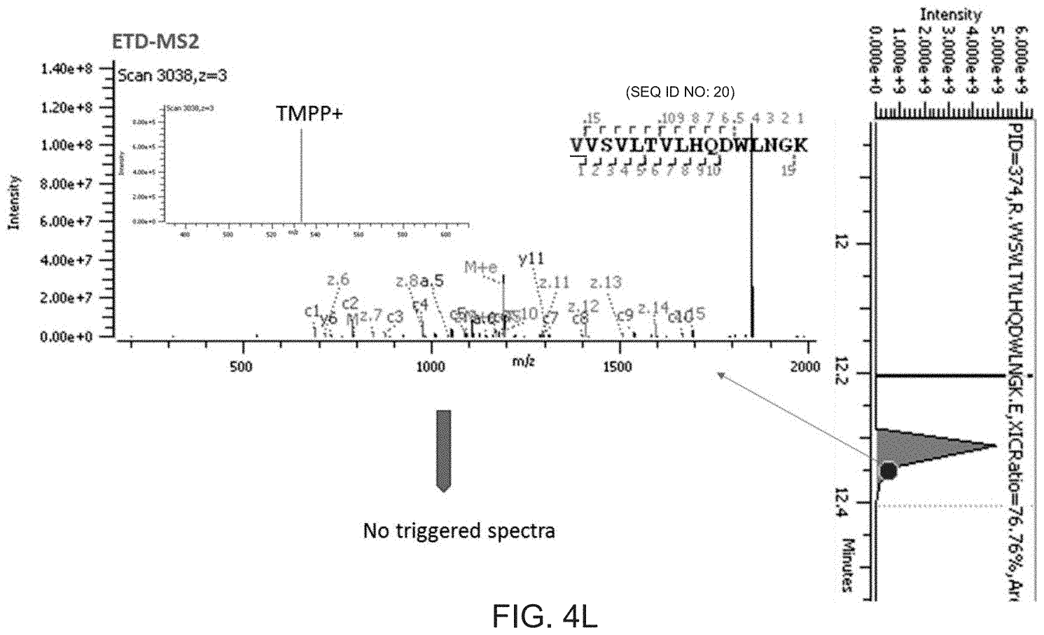

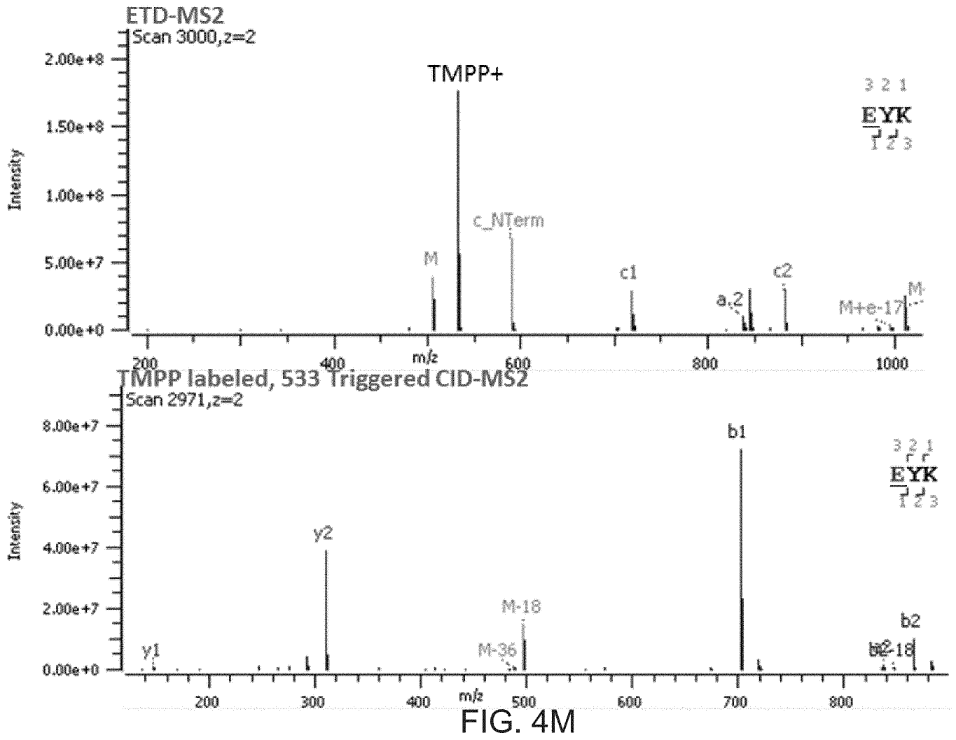

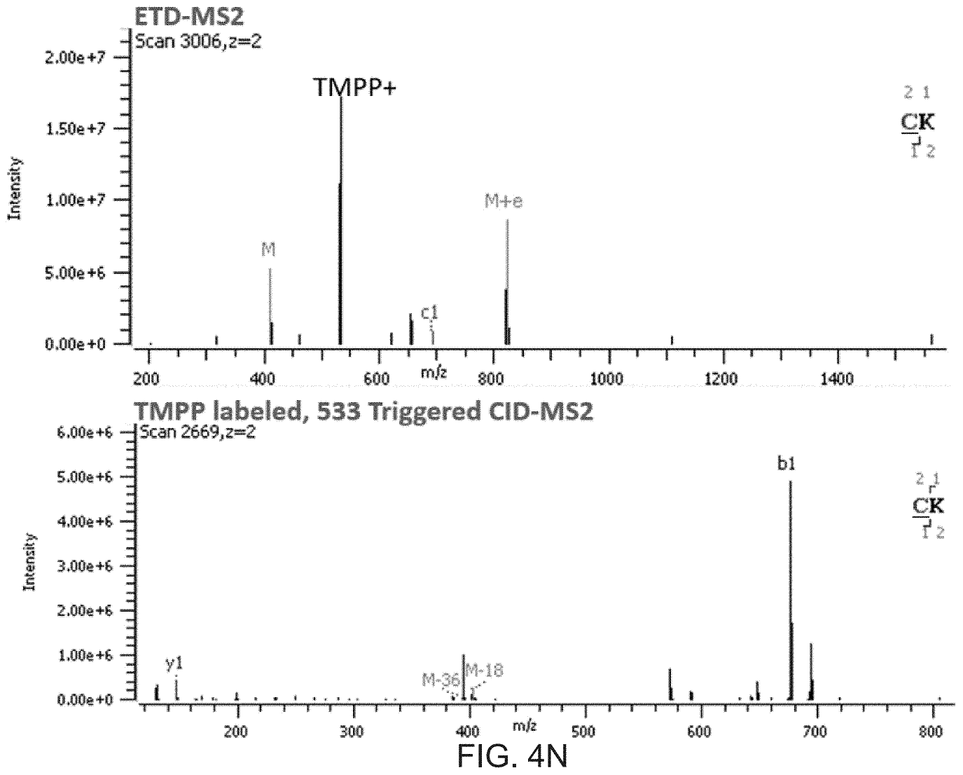

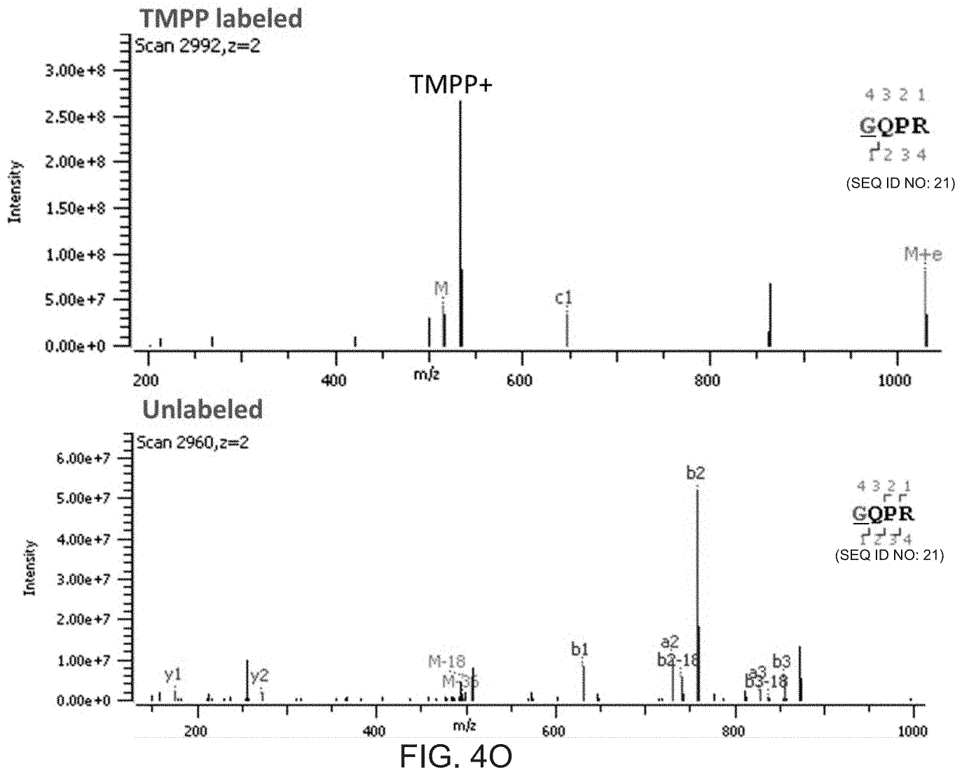

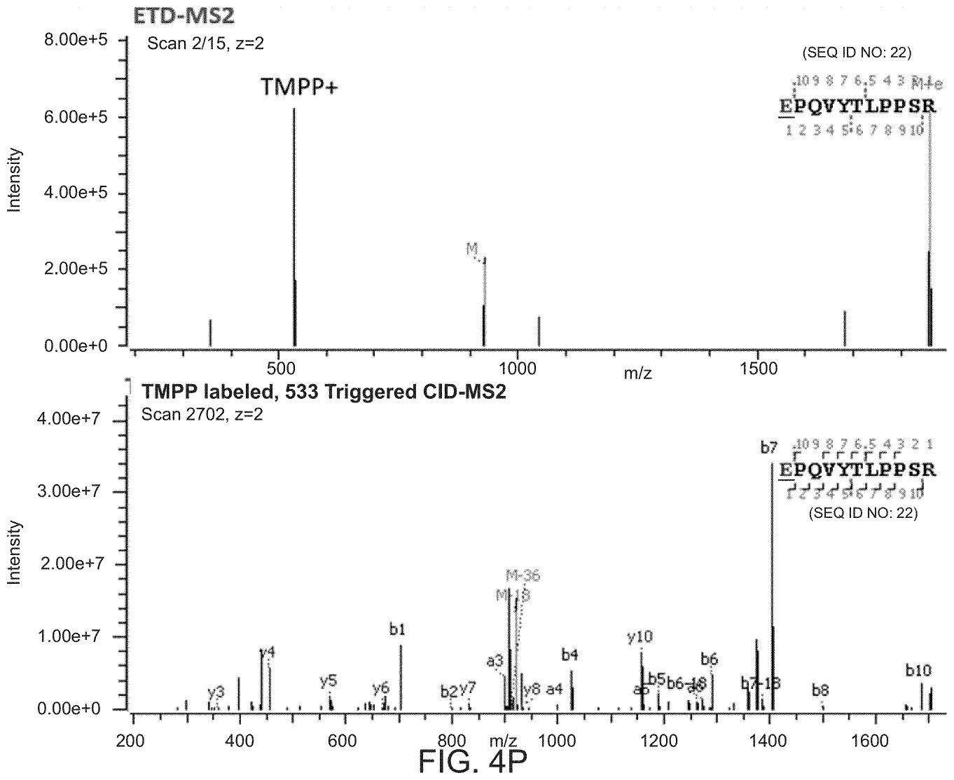

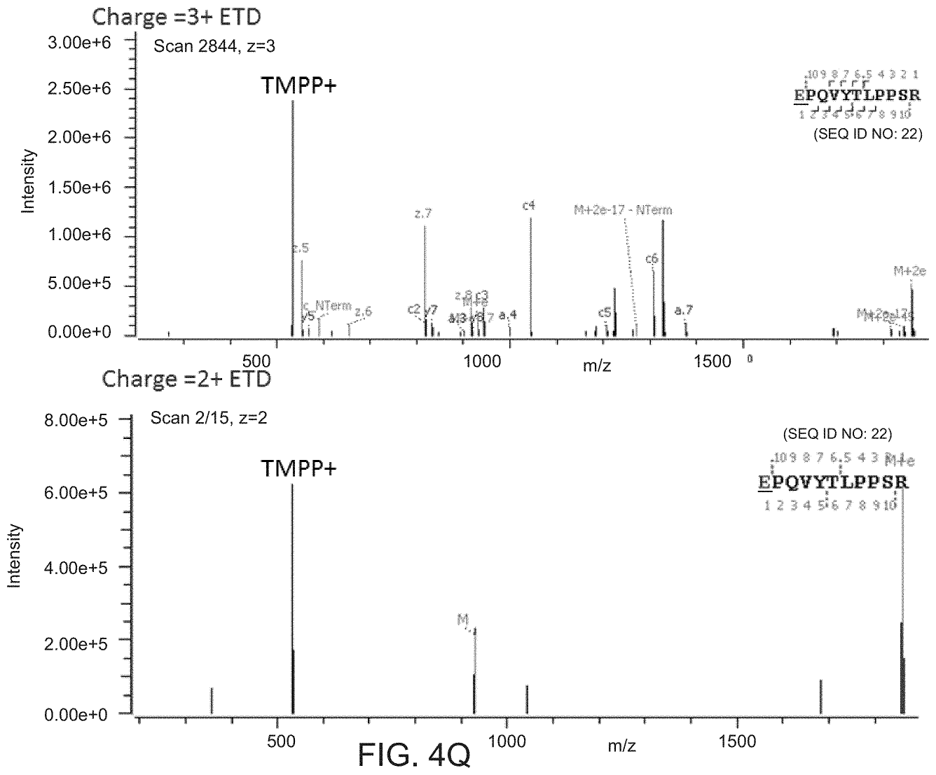

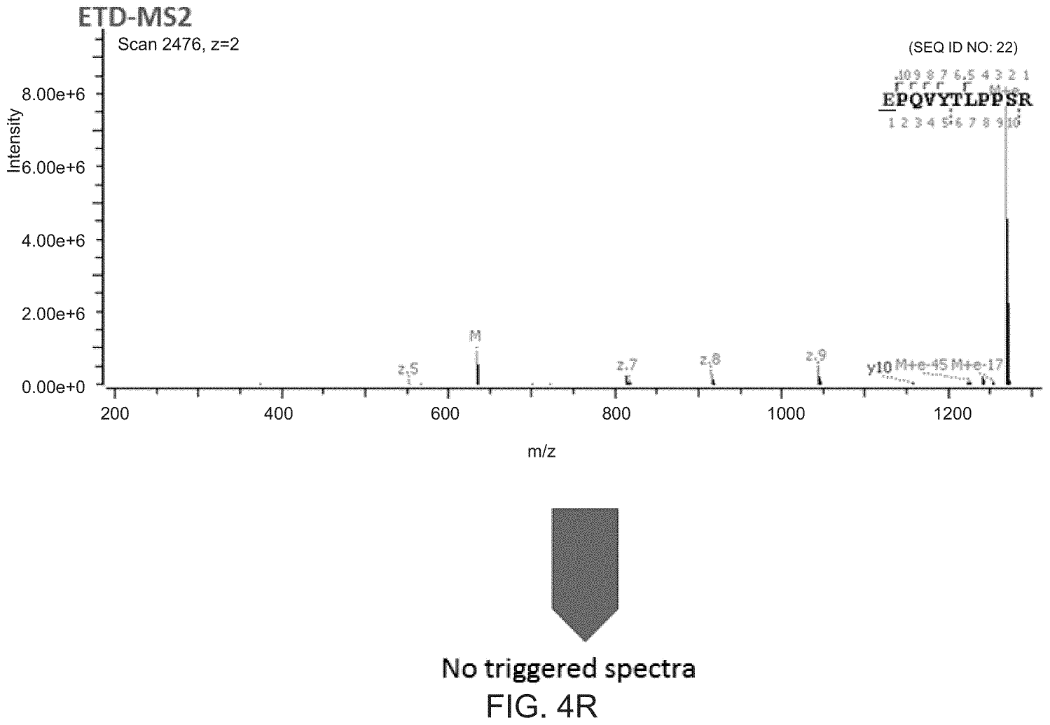

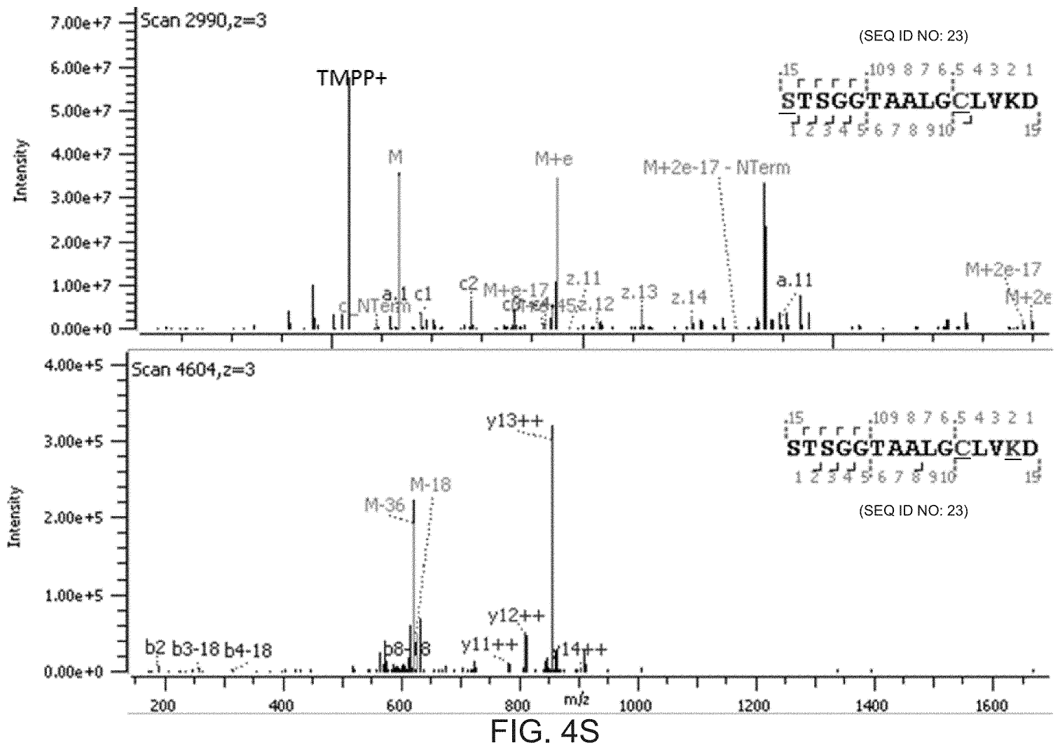

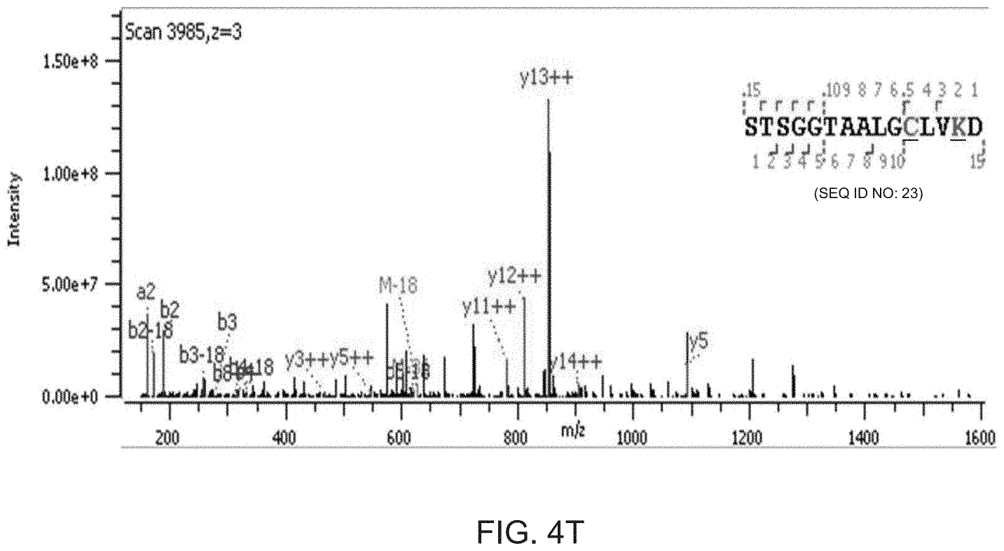

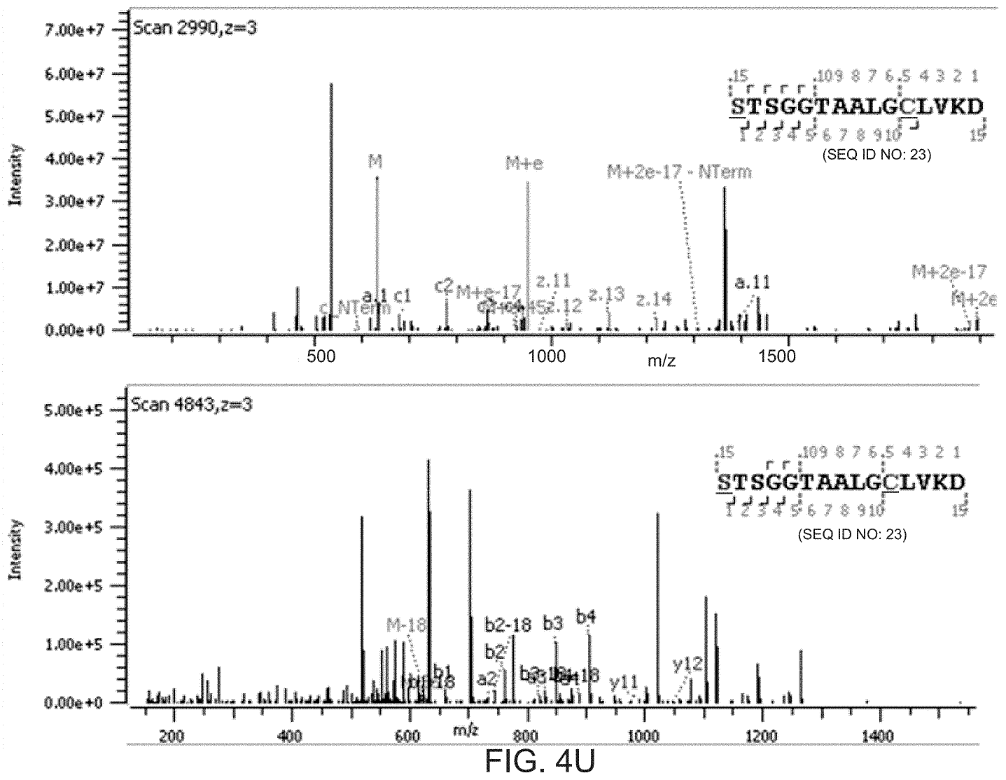

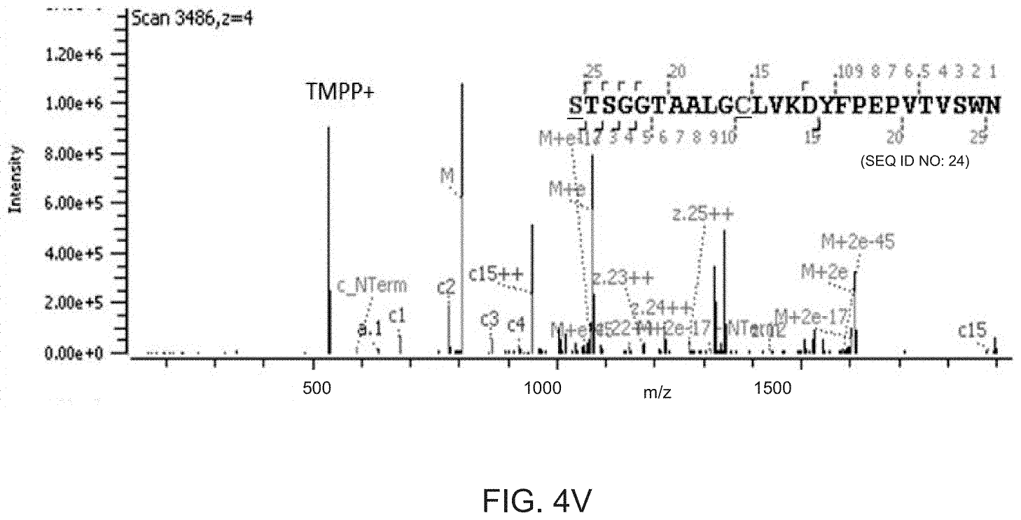

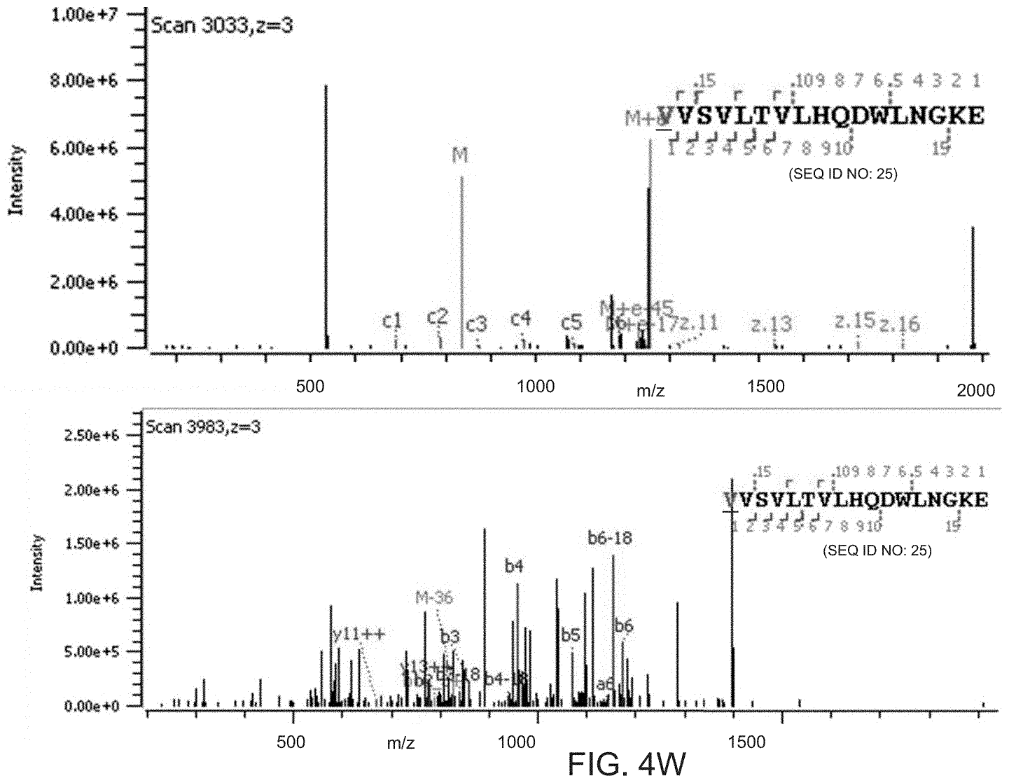

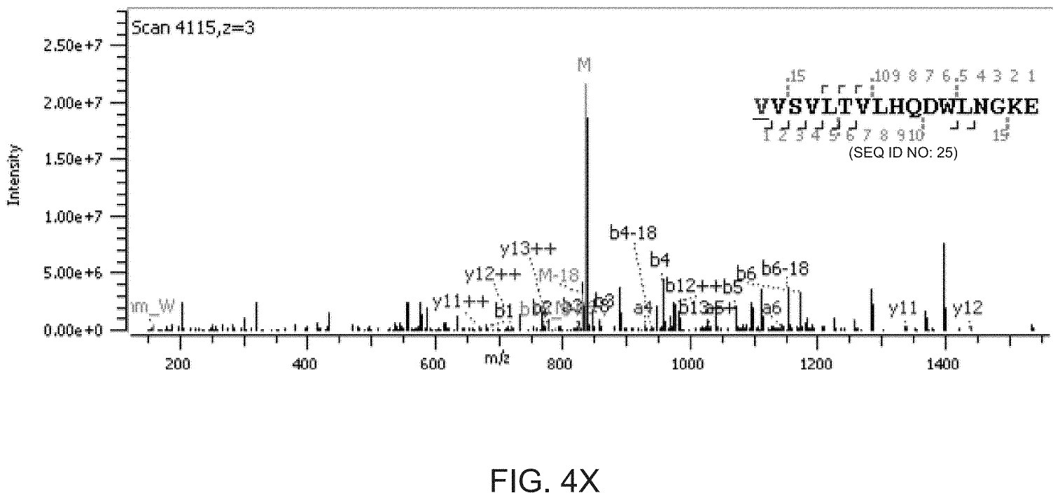

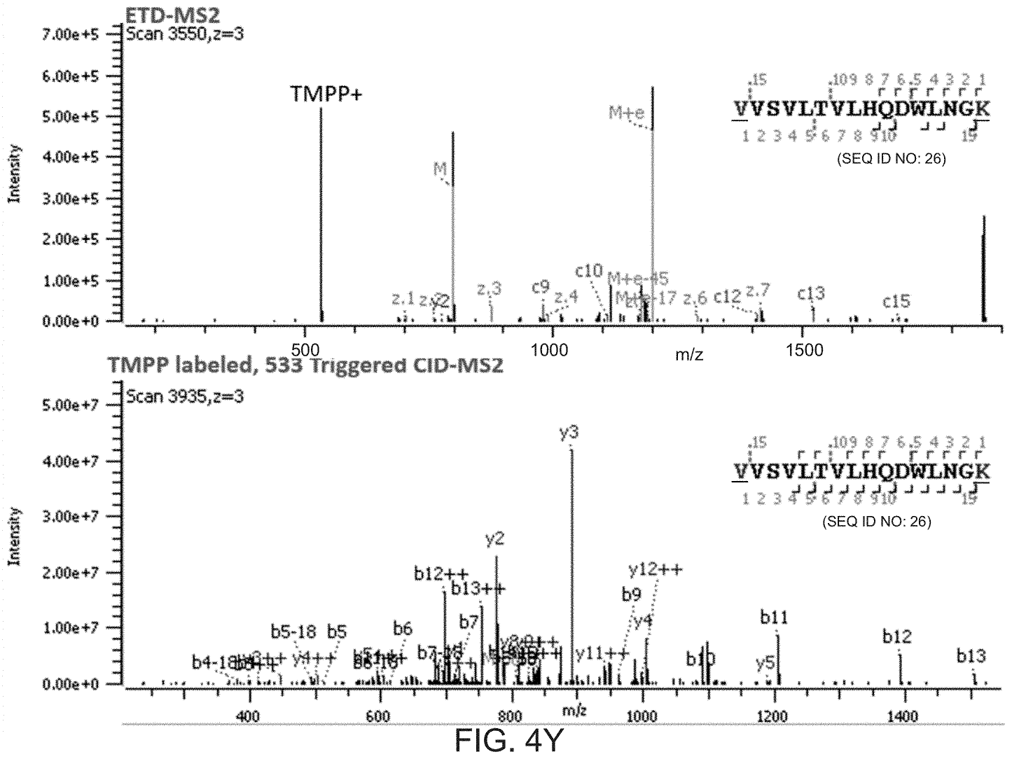

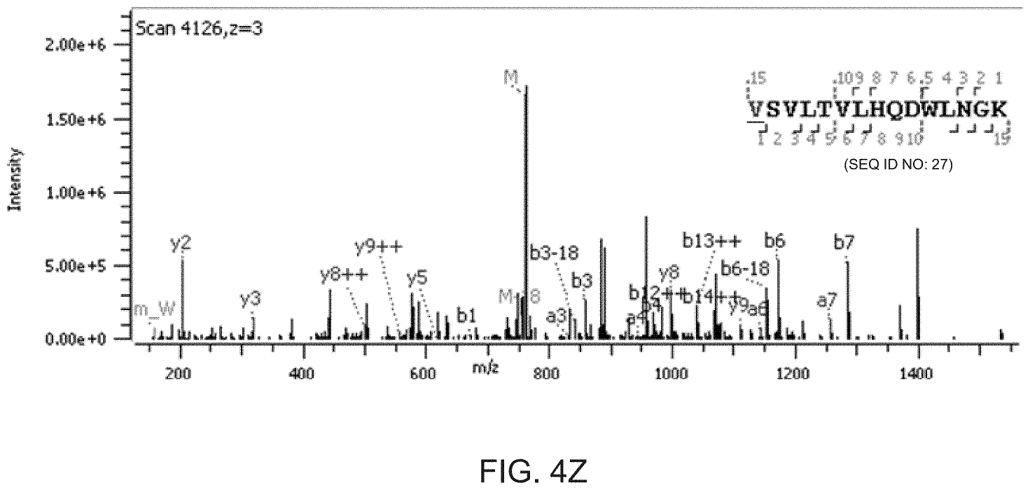

See FIGS. 4A-Z for the related ETD-MS² and TMPP⁺ triggered CID-MS² spectrum. It was also noted that the fidelity of producing a subsequent mass-triggered MS² scans is likely to be impacted by data-dependent criteria where a decision of a subsequent scan was made on the overall intensity of the reporter ion and the AGC settings for a precursor ion (Bowers et al., Scientific Reports, 2018, 8:10399). For example, both peptides FNWYVDGVEVHNAK (SEQ ID NO: 17) and VVSLTVLHQDWLNGK (SEQ ID NO: 26) labeled at N-terminus produced lower intensity TMPP⁺ diagnostic ions. However, mass triggering was observed only for FNWYVDGVEVHNAK (SEQ ID NO: 17; FIG. 4I) while VVSLTVLHQDWLNGK (SEQ ID NO: 26) peptide lack of MS² triggering (FIG. 4L). When subsequent MS1 intensity was examined, peptide VVSLTVLHQDWLNGK (SEQ ID NO: 26) fell below the threshold for MS/MS. Some interesting dissociation behavior based on the of sequence composition of the derivatized peptides was also observed. A single residue extension or the position of the TMPP labels showed a significant effect on the gas-phase dissociation of TMPP⁺ as a charged species. For example, TMPP derivatized at both N-terminus and lysine side chain of VVSLTVLHQDWLNGK (SEQ ID NO: 26; FIG. 4Y) peptide generated a dominant reporter ion upon ETD and a triggered MS2-CID spectrum. It was also observed that peptide VVSLTVLHQDWLNGKE (SEQ ID NO: 25; FIG. 4W) with the addition of Glutamic acid residue at the C-terminus having TMPP at the N-terminus also produced an abundant diagnostic reporter ion and a triggered MS2-CID spectrum. Although not bound by any particular theory, it was hypothesized that the propensity to generate reporter ions via ETD is likely to depend on number of different factors, such as the position of TMPP label, amino acid composition, and charge state of the peptide. Synthetic peptide data suggested that TMPP labeling occurred mostly at the N-terminus with tyrosine and lysine residue derivatization occurring to a lesser extent (i.e., 14 of the 15 N-termini, 1 of the 6 had tyrosine residues and 1 of 10 lysine residues were TMPP labeled).

It was expected that the solvent accessibility of polar residues of intact proteins can lead to undesired labeling of lysine and tyrosine residues as previously reported (Abello et al., Journal of Proteome Research, 2007, 6:4770-4776). However, the triggered MS² approach facilitated the localization on TMPP modifications effectively and helped in the unambiguous determination of clip sites. TMPP labeled lysine containing peptides were unambiguously localized on the N-terminus exclusively by ETD-MS² while diagnostic ion triggered CID-MS² data complemented the ETD spectra and improved the confident localization.

Most of the TMPP labeled tyrosine containing peptides were localized using ETD-MS² with ion-triggered CID-MS² spectra improving the confident localization. However, ETD-MS² alone can be uninformative when sequence ions were insufficient to localize the site of TMPP modification. FIG. 4D shows the annotated ETD-MS² spectrum of a single TMPP labeled peptide VYACEVTHQGLSSPVTK (SEQ ID NO:13) where the search engine had incorrectly assigned TMPP modification on the N-terminus. However, based on the c- and z-type ETD ions, TMPP moiety cannot be unambiguously assigned to either the N-terminus or side chain derivative of tyrosine. The utility of the subsequent triggered MS2-CID scan produced a confident y16 ion that unambiguously localized the TMPP on the tyrosine residue. FIG. 4P shows ETD-MS² spectrum of a single TMPP labeled peptide EPQVYTLPPSR (SEQ ID NO: 22) that exclusively produced the diagnostic ion with no backbone sequence ions. The identification of the sequence was based on the m/z of the precursor ion of the MS1 spectrum while the reporter ion was indicative that the peptide is likely to carry a TMPP moiety. The triggered MS2-CID spectrum generated several backbone ions: y7, y8, and y10 that lacked TMPP modification together with b1-b4 ions that had a TMPP moiety localized the TMPP on the N-terminus. It was important to note that after examining all the MS2-CID spectra, only a few spectra showed diagnostic ions at 573 with limited diagnostic utility.

The complementary nature of how ETD-MS² and triggered CID-MS² was used in these experiments helped to rapidly screen potential clipped species irrespective of the amino acid sequence of a surrogate proteolytic peptide containing the TMPP moiety. The herein described tandem MS approach was amenable to ETD-MS² of peptides with various lengths and charge states and was also effective when peptides were as small as dipeptides that were doubly charged. The ability to produce a dominant diagnostic immonium ion of the TMPP moiety was critical for ETD-MS² generated reporter ions for triggering subsequent CID scan. ETD-MS² has shown to generate TMPP diagnostic ions quite consistently while both ETD and CID backbone fragment ions localized the TMPP modification to a single residue.

### Example 4: Diagnostic Ions of TMPP Labeled Synthetic Derived from Cell Lysate

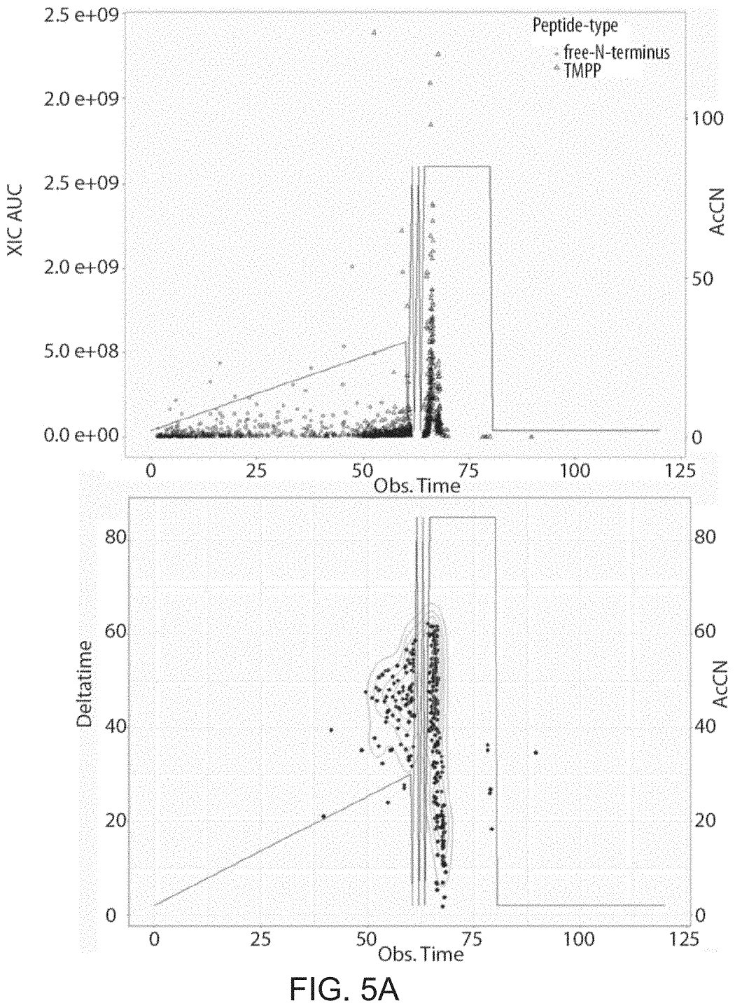

The diagnostic utility of TMPP⁺ reporter ions generated using ETD- and HCD-type fragmentation by examining the efficiency of generating reporter ions for a large pool of tryptic peptides and their TMPP derivatives derived from a K562 cell lysate were next examined. The complexity of peptides required changes to the overall LC separation time and therefore peptides were subjected to two single-shot 6× longer run times (120 min) with ETD-MS² and HCD-MS² dissociation performed separately for each run. FIG. 5A shows peptide intensities as a function of observed retention times overlaid with the AcCN gradient. The unlabeled peptides eluted up to ˜30% AcCN as expected, while TMPP labeled peptide eluted adjacent to the two rapid organic ramps (0-85% AcCN) like the shorter gradient runs. The observation of TMPP labeled synthetic standard peptides with higher overall intensity distribution was indicative of increased hydrophobicity of the peptides and improved ionization of the TMPP labeled peptides. FIG. 5B shows the observed time distributions of the subset of TMPP labeled peptides that had a corresponding unlabeled peptide. Next, the observed time difference between the labeled and corresponding unlabeled peptides (Delta time) was shown as function of the observed retention time overlaid with the AcCN gradient. The TMPP labeled sequence eluted later for almost every peptide as given by a large positive value. The density of TMPP labeled peptides were seen at high retention times at the two rapid AcCN ramps of the gradient and at high delta time.

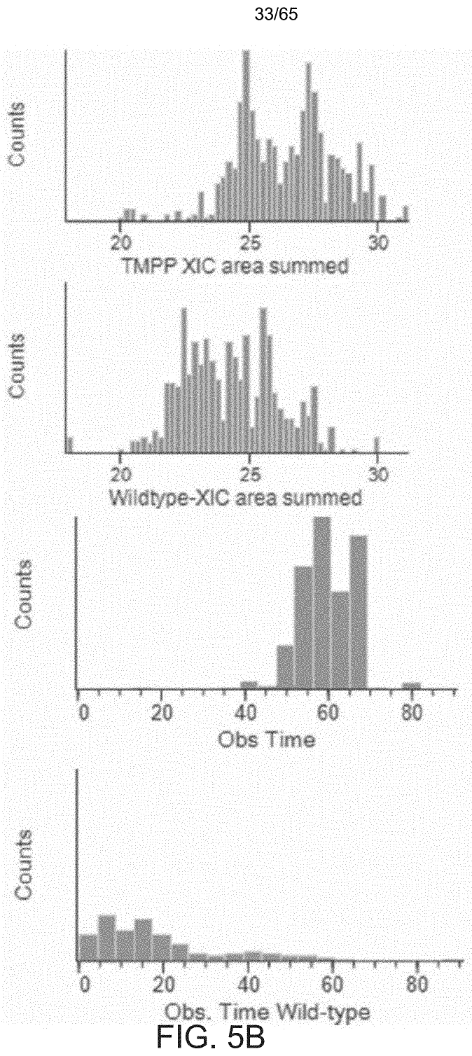

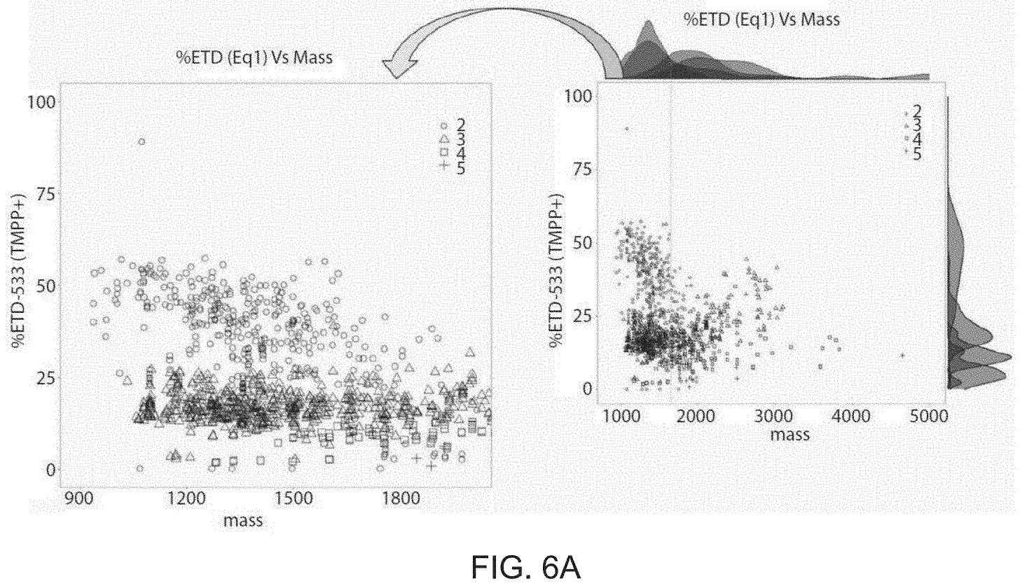

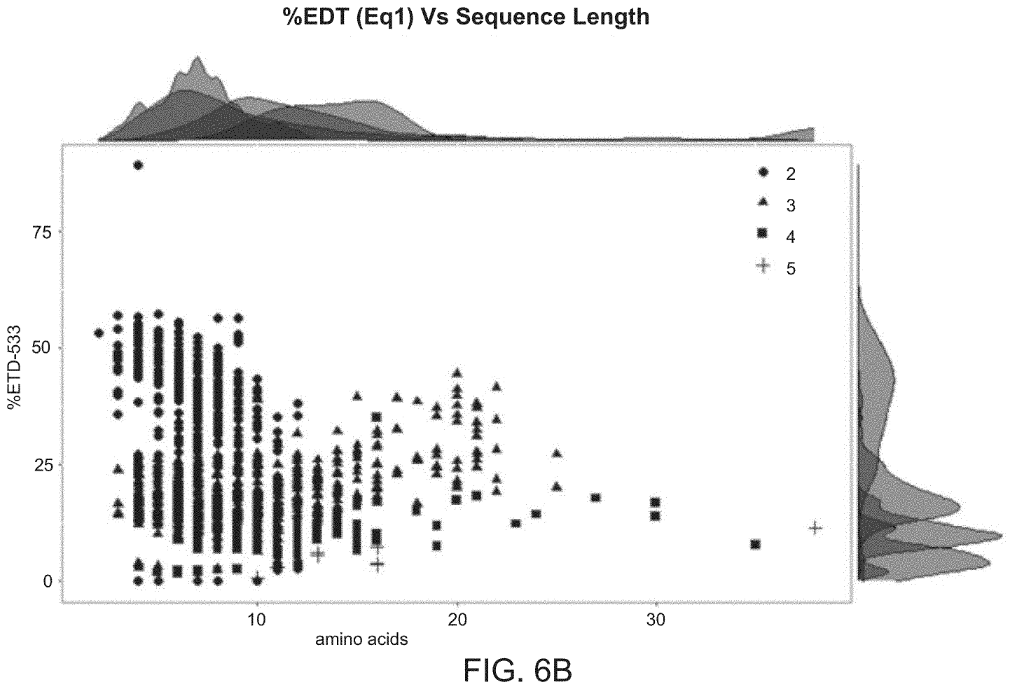

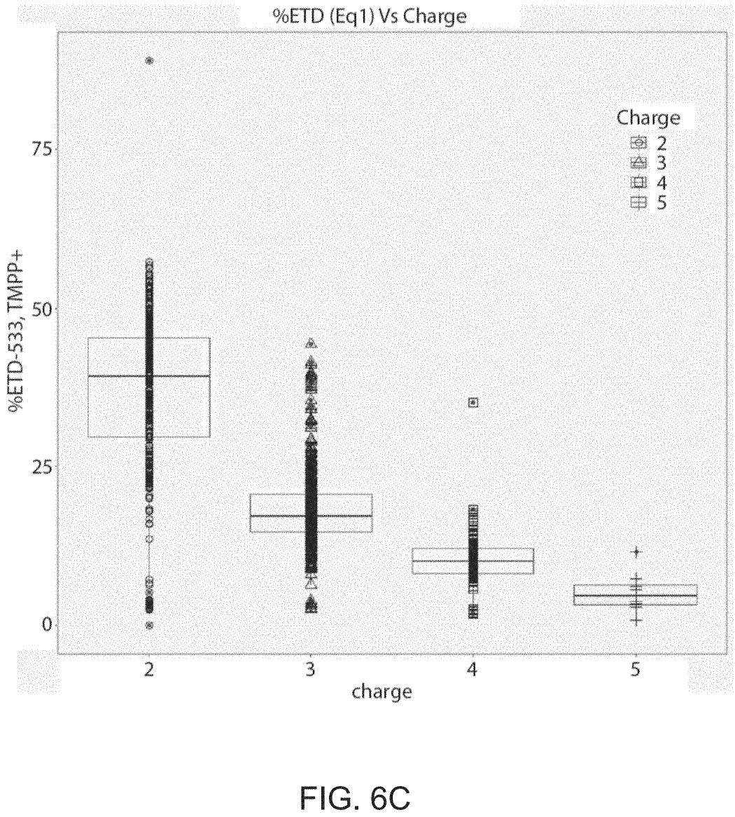

The propensity to generate diagnostic TMPP reporter ions (m/z=533 Da, and 590 Da) in the ETD spectra and TMPP reporter m/z 573 Da for HCD spectra were carefully evaluated for peptides having different lengths, amino acid composition, charge state, and sites of TMPP labeling. The efficiency of generating diagnostic ions for every TMPP labeled peptides was estimated using several different methods. The reporter ion intensity was normalized to various types of product ion intensities as shown by the relations in Eq1-Eq3 (see FIG. 2D) for both types of ETD derived reporter ions: TMPP⁺ and TMPP-AC-NH₂⁺. In almost all peptides, TMPP⁺ reporter ion abundance was predominant over TMPP-Ac-NH₂⁺ (FIG. 6A through FIG. 6C).

$$\% ETD ( TMPP + ) = \frac{{( TMPP + )}_{}}{{( All ions )}_{}} \qquad Eq 1$$

$$\% ETD ( TMPP + ) = \frac{{( TMPP + )}_{}}{{( 590 and C and Z ions only )}_{}} \qquad Eq 2$$

$$\% ETD ( TMPP + ) = \frac{{( TMPP + )}_{}}{{( All ions Except precursor or 3 + )}_{}} \qquad Eq 3$$

$$\% ETD ( TMPP - Ac - NH 2 + ) = \frac{{( TMPP - Ac - NH 2 + )}_{}}{{( All ions )}_{}} \qquad Eq 1$$

$$\% ETD ( TMPP - Ac - NH 2 + ) = \frac{{( TMPP - Ac - NH 2 + )}_{}}{{( 533 and C and Z ions only )}_{}} \qquad Eq 2$$

$$\% ETD ( TMPP - Ac - NH 2 + ) = \frac{{( TMPP - Ac - NH 2 + )}_{}}{{( All ions Except precursor or 3 + )}_{}} \qquad Eq 3$$

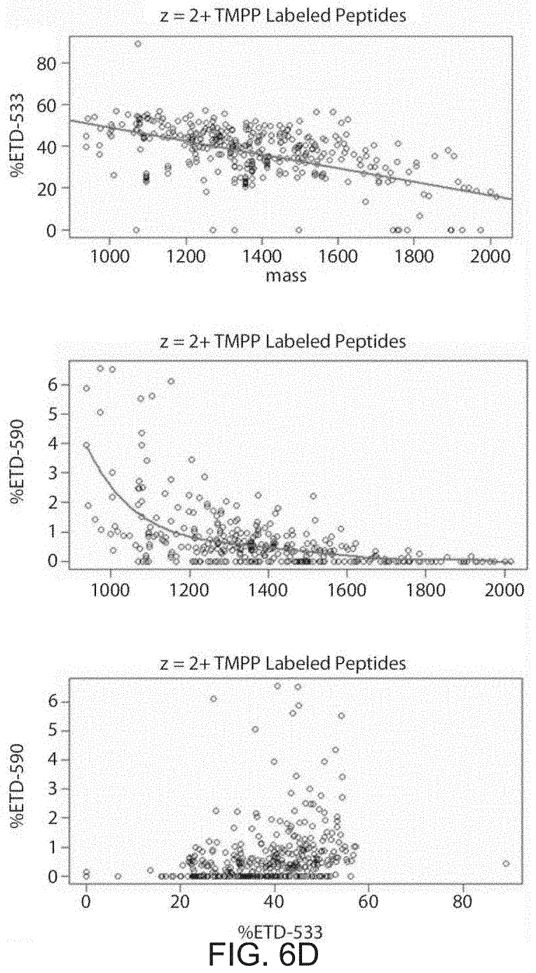

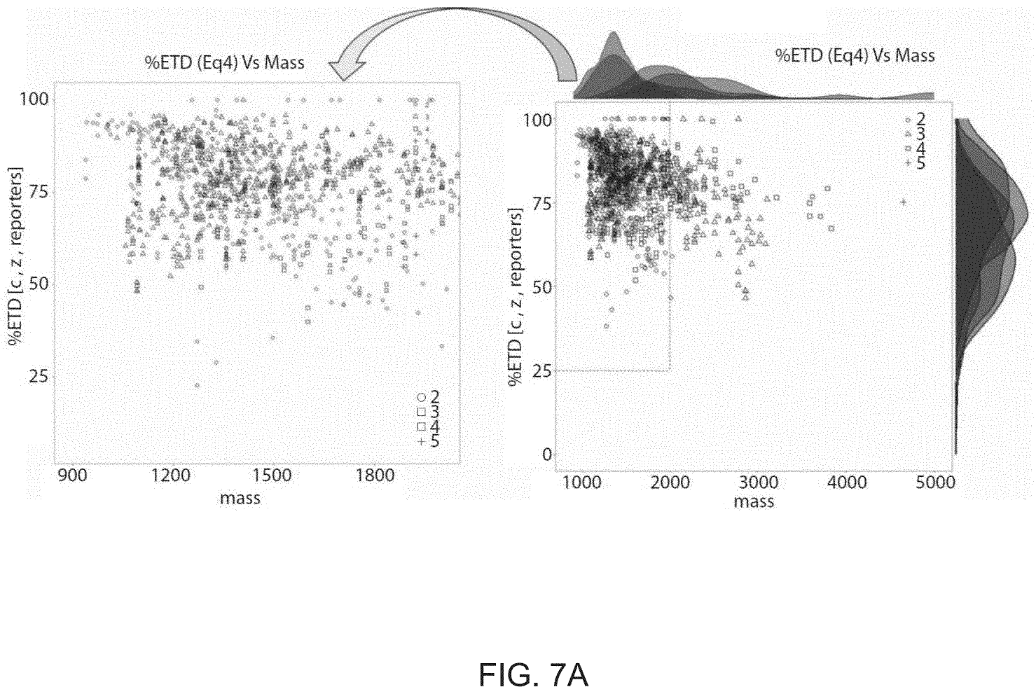

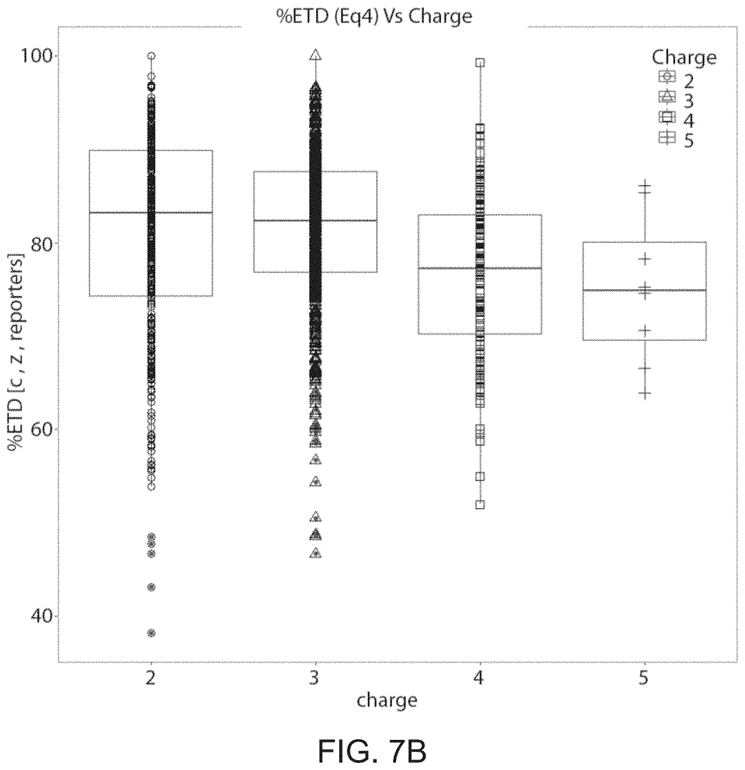

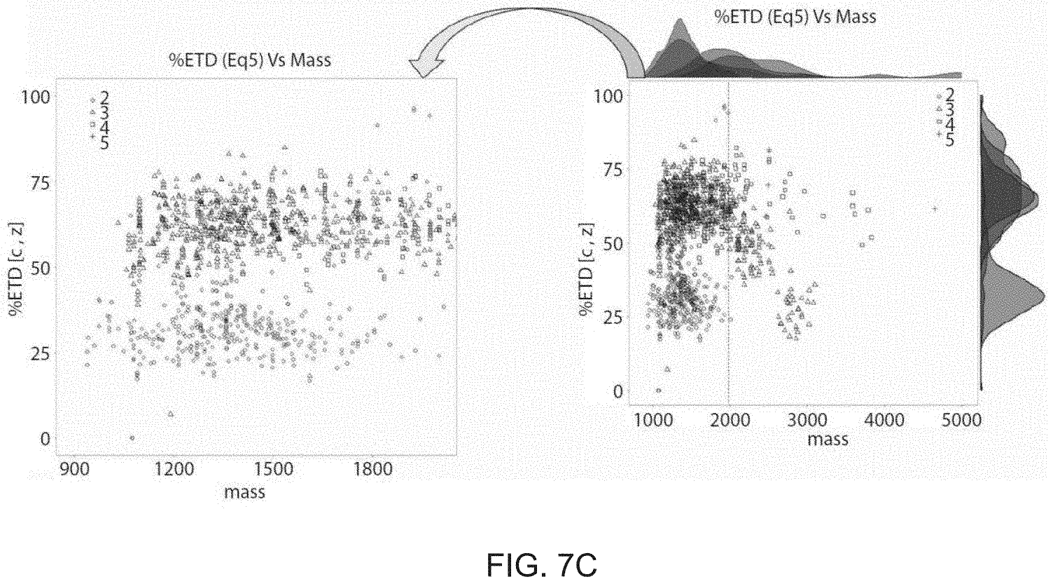

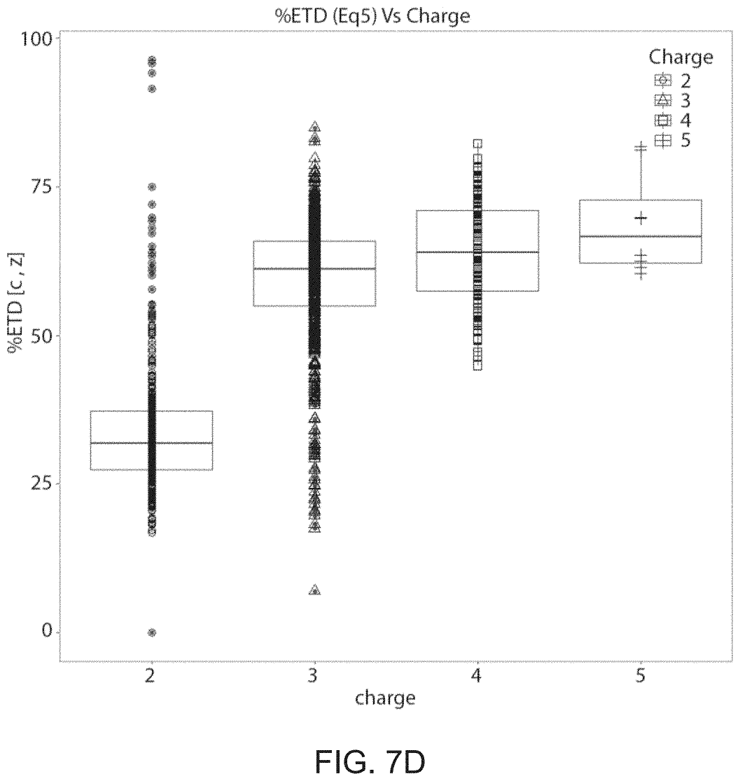

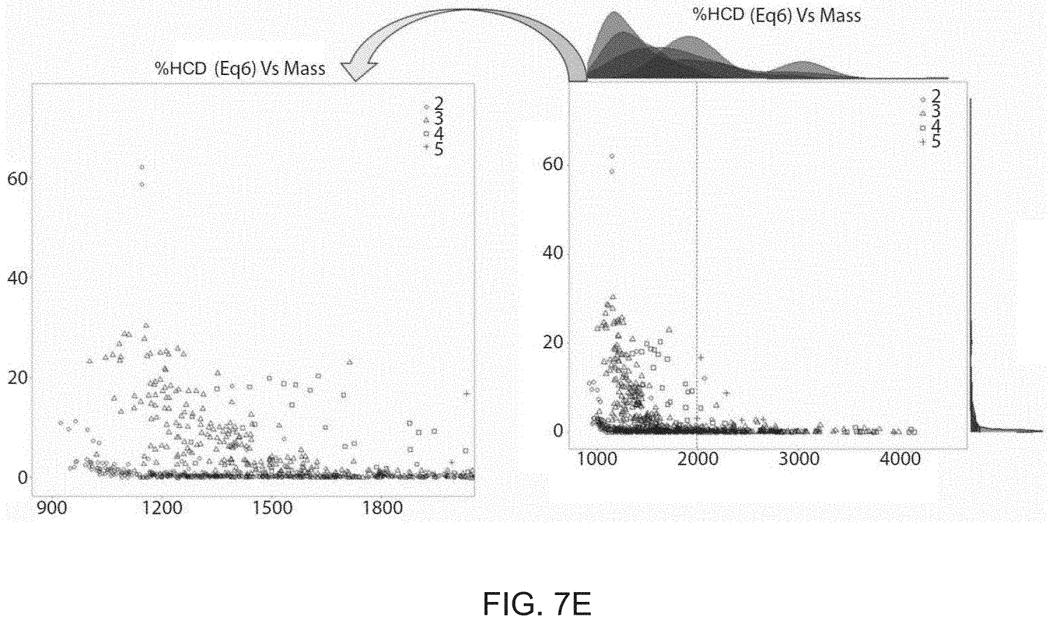

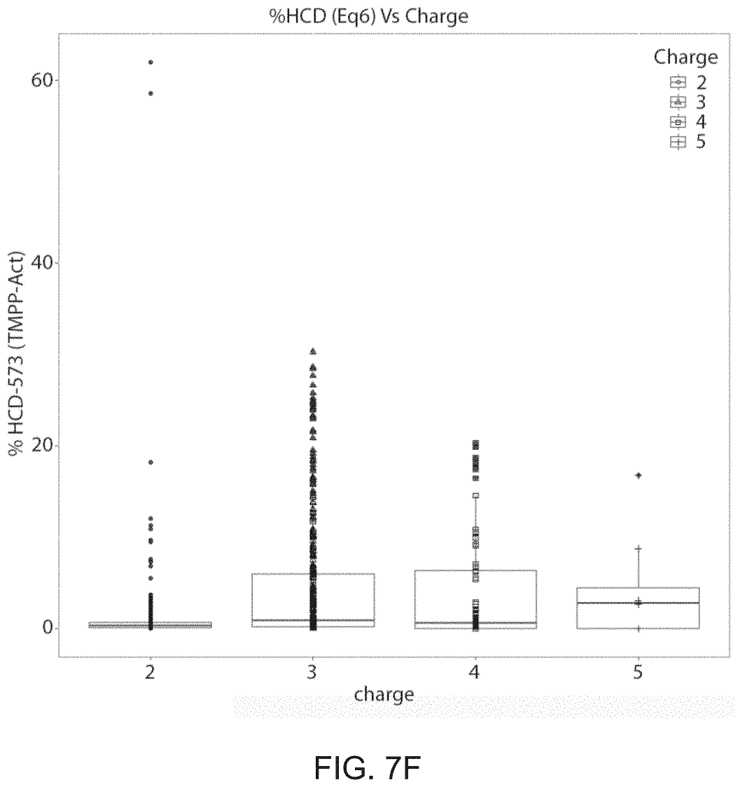

In addition, the results for the overall ETD efficiency for all backbone reporters and C, Z type product ions as shown in Eq4 (Gunawardena et al., Journal of the American Chemical Society, 2005, 127:12627-12639) and all backbone fragments, except the reporter ions as shown in Eq5, were examined. Analogous to ETD reporter ion estimation in Eq1, reporter ion derived from HCD was normalized to product ions as shown in Eq6 (see FIG. 2E). As indicated in FIGS. 5A-5B, the TMPP⁺ (533 Da) efficiency was reported as a function of peptide precursor mass or peptide length grouped by the charge state of the precursor. Furthermore, a charge state dependency on the ETD efficiency for TMPP⁺ reporter ion was important to note. It was observed that TMPP⁺ efficiency was highest in doubly charged precursor ions and decreased linearly with the mass of the peptide. Also, TMPP⁺ efficiencies were significantly higher than the TMPP-Ac-NH₂⁺ (590 Da). In general, the propensity to produce TMPP⁺ reporter ions favored doubly charged precursor over triply charged precursor ions for peptides with similar mass or same number of amino acids. This observation was interesting as backbone c and z fragment ions of these same peptides showed increased efficiency with increased charge states (e.g., FIG. 7A through FIG. 7D).

$$\% ETD = \frac{{( C , Z ions and Reporters )}_{}}{{( All ions Except precursos or 3 + )}_{}} \qquad Eq 4$$

$$\% ETD = \frac{{( C , Z ions )}_{}}{{( All ions Except precursos or 3 + )}_{}} \qquad Eq 5$$

Then the overall backbone ETD efficiency of TMPP labeled peptides considering all backbone fragment ions and all backbone fragment ions, except the reporter ions, were evaluated. As indicated in FIG. 7A, the overall ETD efficiency, considering all product ions, showed a subtle charge state dependent decrease. In contrast, FIG. 7B shows that, for overall backbone efficiency estimated by Eq5 where TMPP⁺ reporter ions were disregarded, the ETD efficiency showed a charge state dependent increase, which was generally observed for unmodified peptides as reported elsewhere. Considering all these observations, the contribution of TMPP⁺ reporter ion intensities to the overall efficiency estimates especially for doubly charged ions was significant. The diagnostic utility of ETD generated TMPP⁺ ions was perfectly suited to tryptic peptides that were mostly doubly charged. As shown in FIG. 7C, the HCD derived TMPP-Ac⁺ (573 Da) efficiency was reported as a function of peptide precursor mass grouped by the charge state of the precursor. Overall, the efficiency of the HCD generated TMPP-Ac⁺ reporter ion was significantly less compared to the ETD generated TMPP⁺ reporter ions. The efficiencies of TMPP-Ac⁺ reporter ion of most peptides was <1% with significant fraction having no diagnostic reporter ions or zero efficiency. There were a few peptides showing extreme efficiencies as high as ˜30% (the two outliers close 60% efficiency were false positive assignments where the precursor m/z was the same as reporter ion m/z). No charge state dependency on the HCD efficiency for TMPP-Ac+ reporter ion was observed.

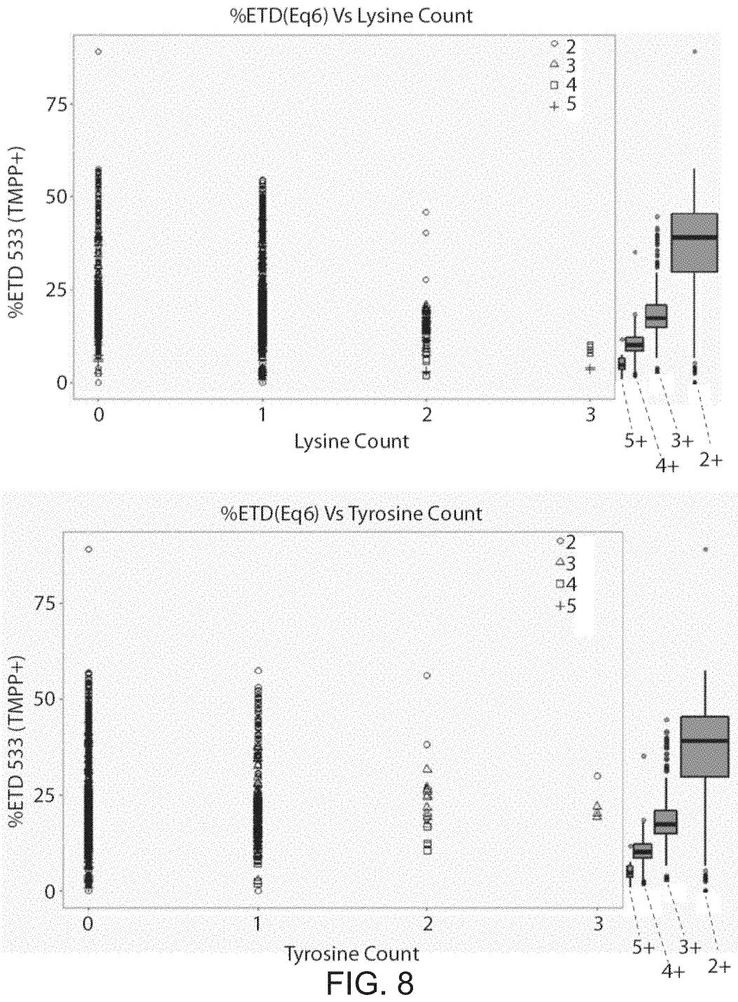

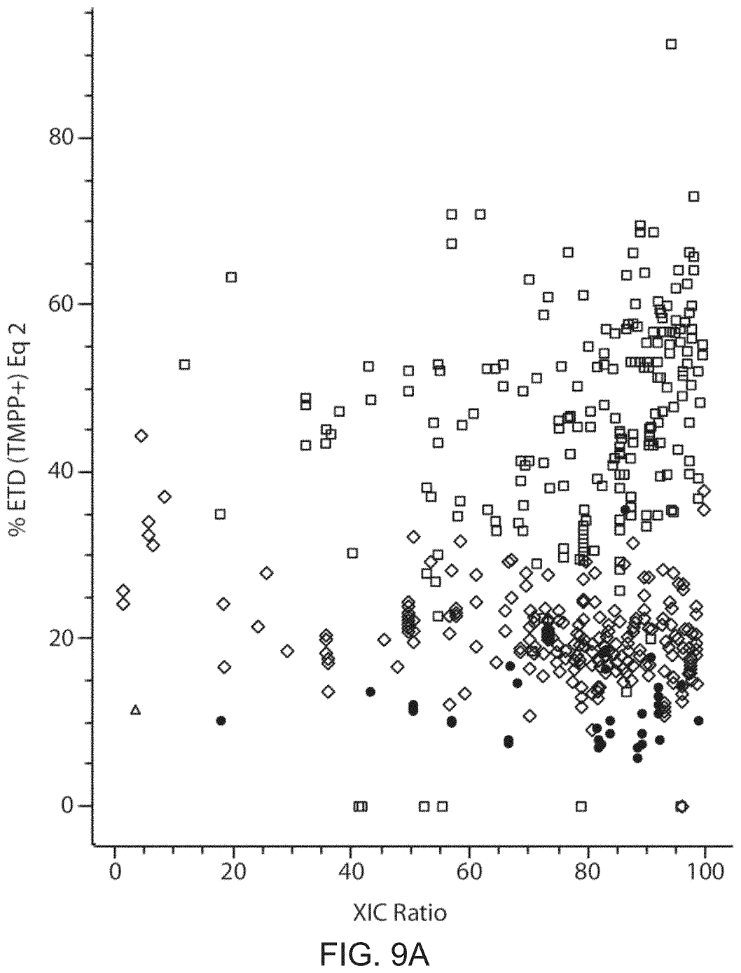

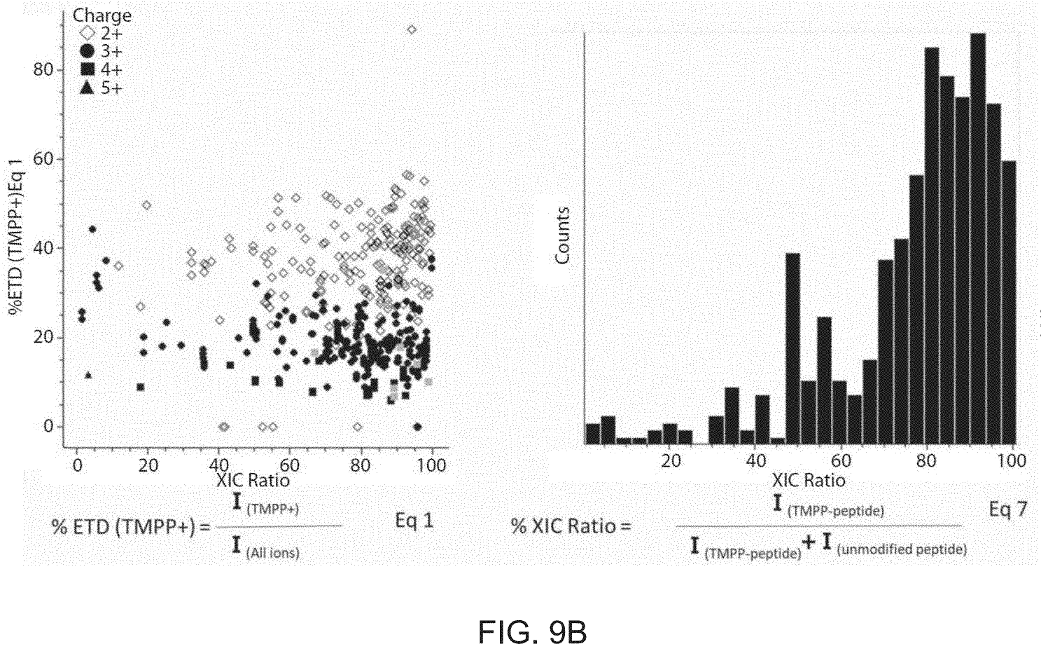

Next, the likelihood of off-labeling of TMPP to lysine and tyrosine residues was examined. 12 PSMs of TMPP labeled at tyrosine residue were identified from a total of 520 tyrosine containing peptide to sequence matches (PSM). From these 520 tyrosine containing PSMs, 262 PSMs had TMPP derivatized N-termini and the remaining 246 PSMs were unmodified. No PSMs of TMPP labeled at lysine residues were identified from a total of 1441 lysine containing PSMs. From the 1441 lysine containing PSMs, 771 PSMs had TMPP derivatized N-termini, 11 PSMs had TMPP derivatized tyrosine, and the remaining 659 PSMs were unmodified. FIG. 8 shows that ETD efficiency has no effect on the labeled peptides grouped by the number tyrosine and lysine residues per peptide. These data suggested that the TMPP labeling of peptides were mostly at N-termini under the described reaction conditions and additional unlabeled lysine or tyrosine residues had no effect on diagnostic TMPP⁺ ion. FIG. 9 shows the overall distribution of the reaction or TMPP labeling efficiency of peptides estimated by Eq7. The derivatized precursor ion subjected to ETD had no bearing on the levels of derivatization. In other words, a peptide derivatized a 100% would have the same ETD efficiency as the same peptide derivatized by 1%. This was important when considering these reactions in the context of clip site identification of proteins, derivatization efficiency at the protein had no consequence on the ETD efficiency of the surrogate peptide.

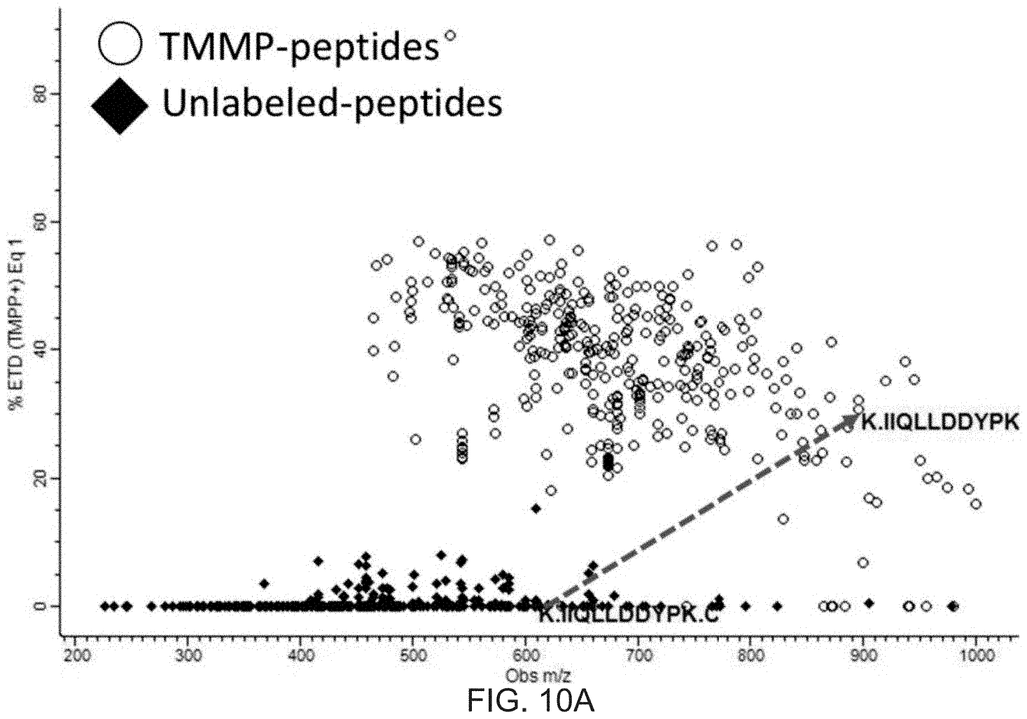

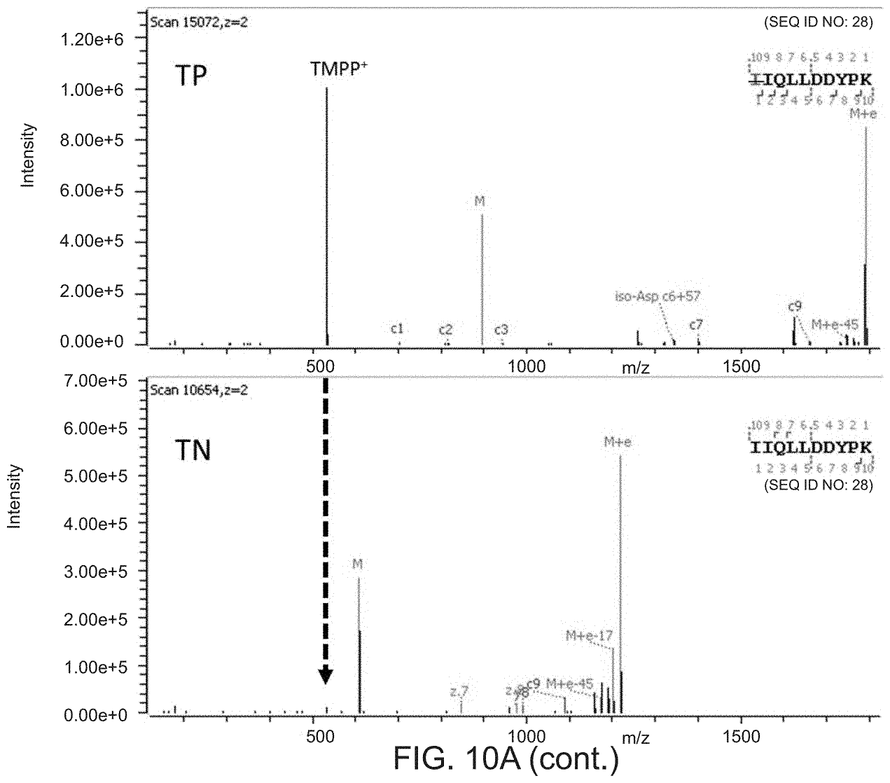

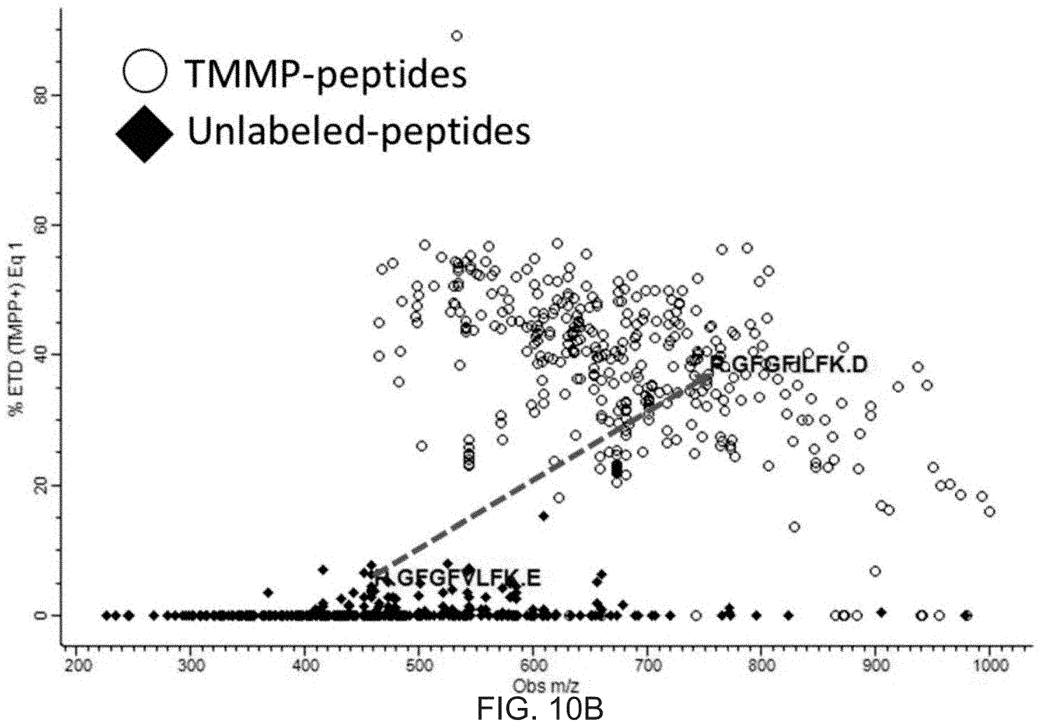

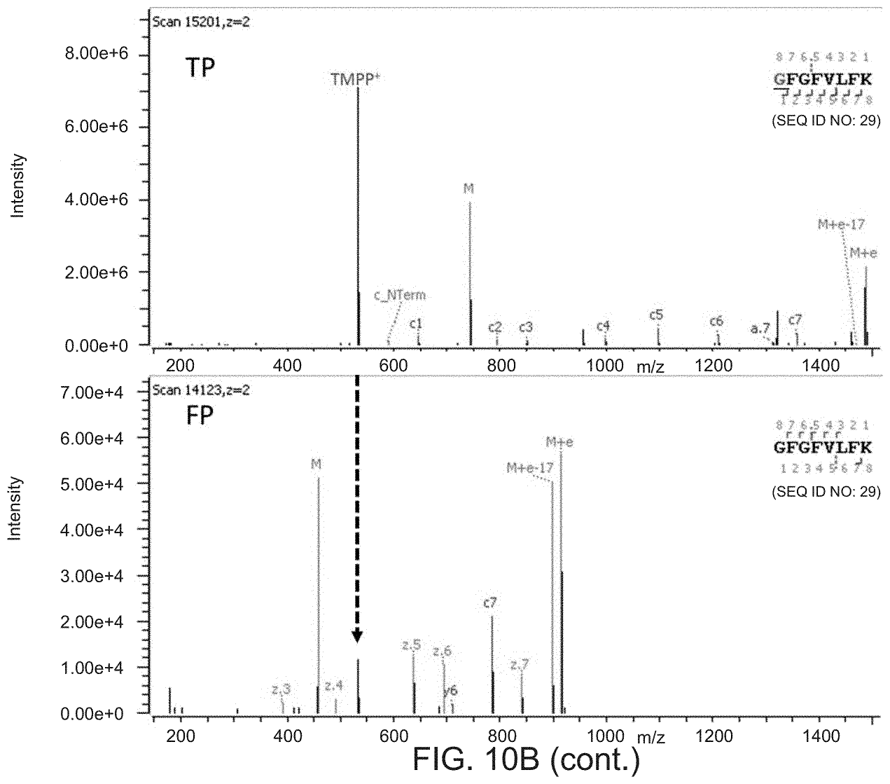

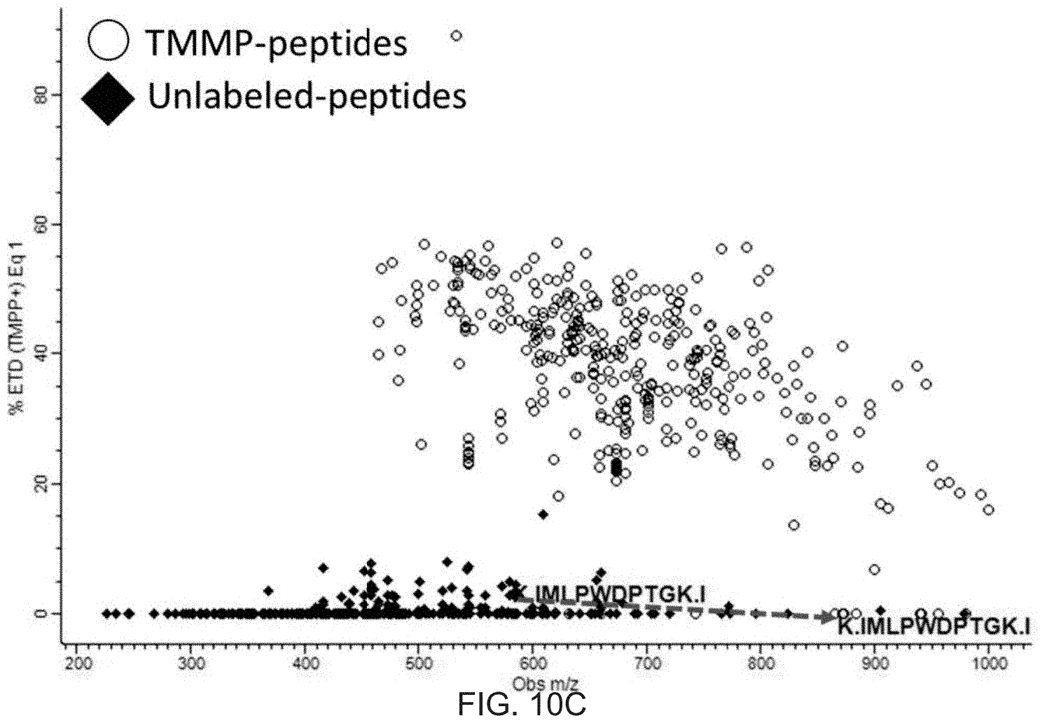

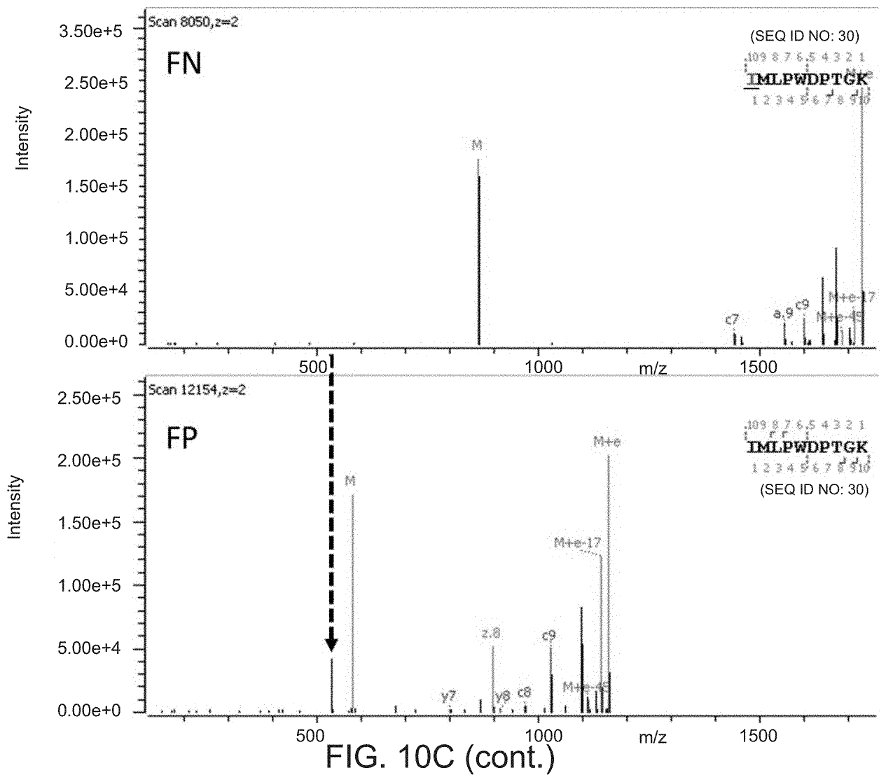

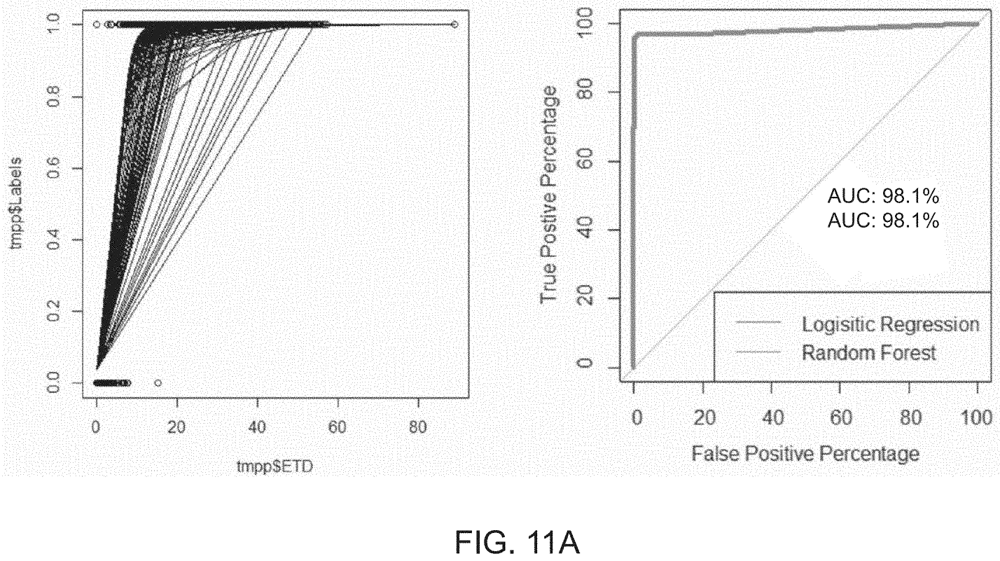

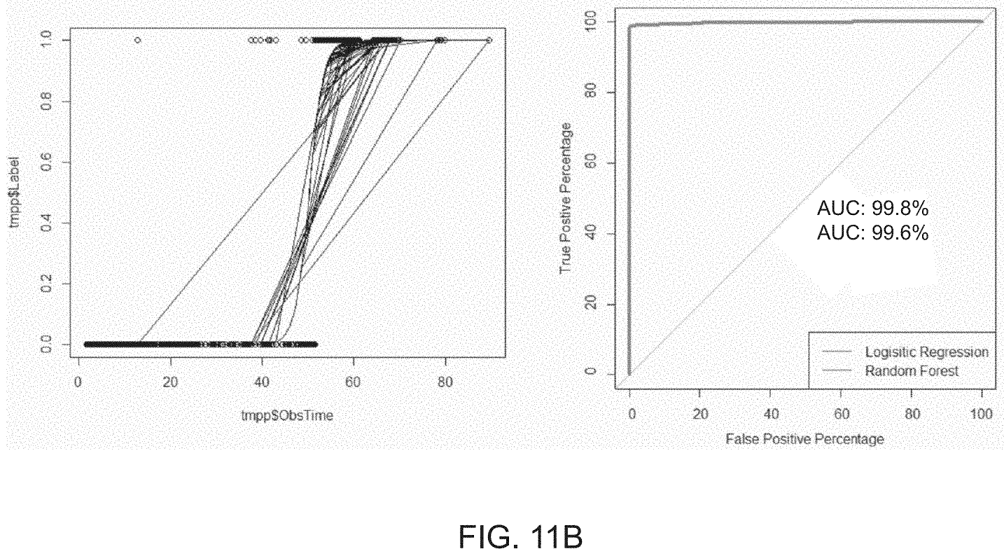

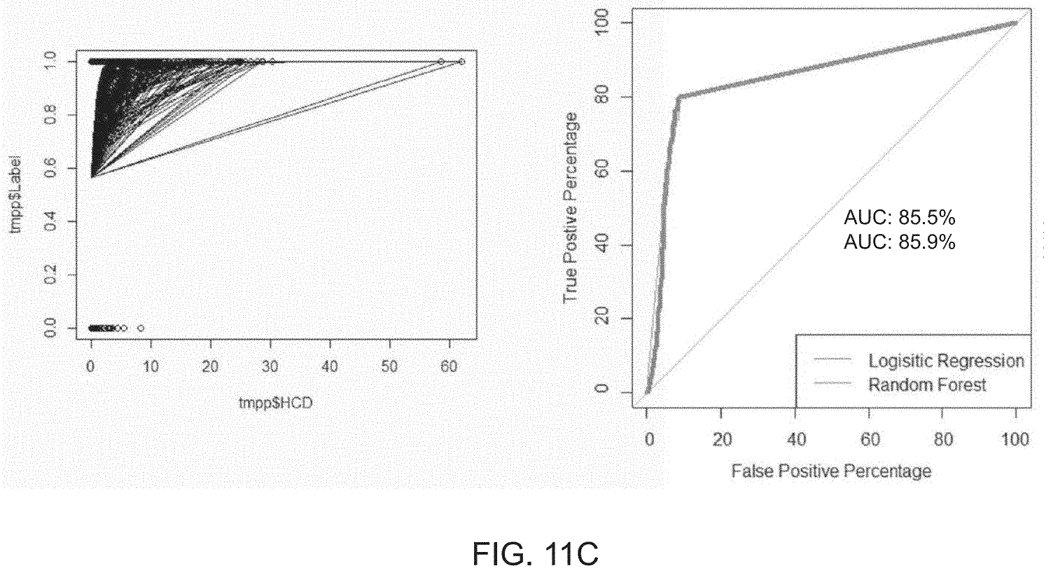

Finally, the diagnostic utility of each reporter ion generated by ETD and HCD and LC retention time was examined. TMPP derivatized peptides and their unmodified counterparts were confidently identified via searches against the human protein sequences, with sequence ions localizing the TMPP moiety with high confidence on mostly N-termini of a peptide. The search results were used to determine and separate the classes of TMPP labeled peptides from unlabeled peptides. Then the dissociation efficiency (ETD or HCD) of the diagnostic ion for each spectrum was subjected to logistic regression and random forest model to determine the sensitivity and specificity of each diagnostic ions to generate receiver operator characteristics (ROC) curves for both ETD and HCD diagnostic ions. In a similar manner, the retention time of both labeled and unlabeled peptides was used to determine the sensitivity and specificity of the elution time. It was illustrated how search results and diagnostic ion abundance is used to classify or misclassify spectra. As indicated in FIG. 10A, the labeled peptide produced a characteristic TMPP⁺ reporter ion, which was a True Positive (TP), while the unlabeled peptide counterpart did not produce a reporter, which was a True Negative (TN). As indicated in FIG. 10B, the labeled peptide produced a characteristic TMPP⁺ reporter ion, which was a True Positive (TP), while the unlabeled peptide counterpart produced an interfering ion similar in mass to the TMPP⁺ reporter ion, which was a False Positive (FP). As indicated in FIG. 10C, the modified peptide did not produce a diagnostic ion, which was a False Negative (FN), while the unmodified peptide counterpart produced an interfering ion similar in mass to the TMPP⁺ reporter ion, which was a False Positive (FP). FIGS. 11A-B show the Area Under the Curve (AUC) of the ROC curve for each diagnostic ion and elution time. Of the reporter ions, TMPP⁺ reporter ions were the most diagnostic with the highest AUC of 98% followed by TMPP-Ac-NH₂⁺ AUC of 85% and TMPP-Ac⁺ AUC of 84%, which suggested that the ETD generated TMPP⁺ diagnostic ions were most accurate and most specific compared to the other reporter ions. The ROC curve of the observed retention time AUC of 99% was most diagnostic of all measures.

### Example 5: Application of TMPP Labeling and ETD Reporter Ion Triggered CID for Detecting Therapeutic Protein Degradation

Finally, the TMPP labels were applied to investigate clipping sites of a commercially available therapeutic, GLP1 agonist, Dulaglutide known to undergo protease induced clipping of the GLP1 peptide. Dulaglutide, a GLP1-Fc fusion protein treated with cathepsin D, was used to study putative clip sites on the GLP1 peptide using ETD-MS² and diagnostic ion triggered CID-MS².

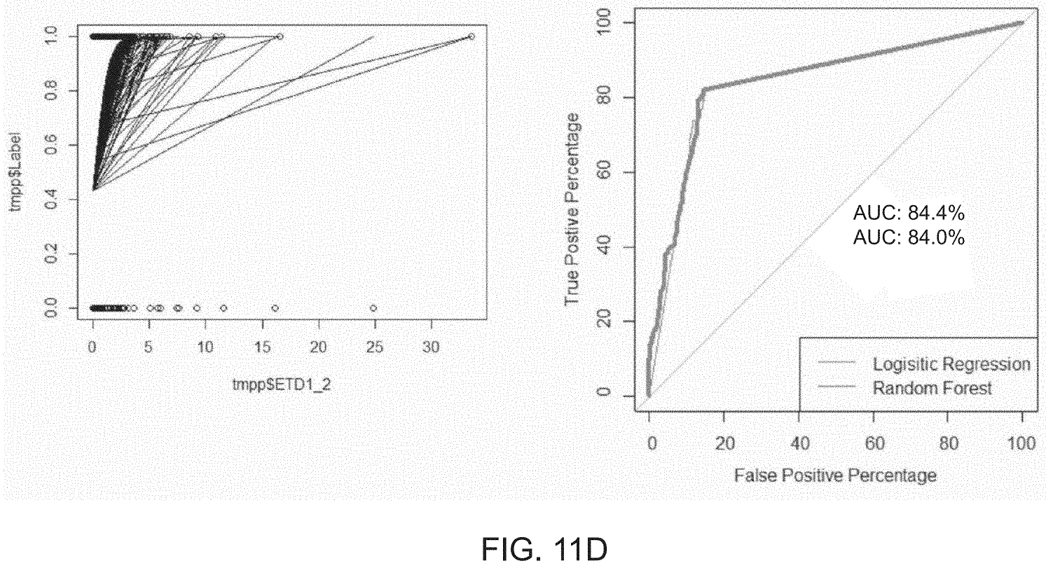

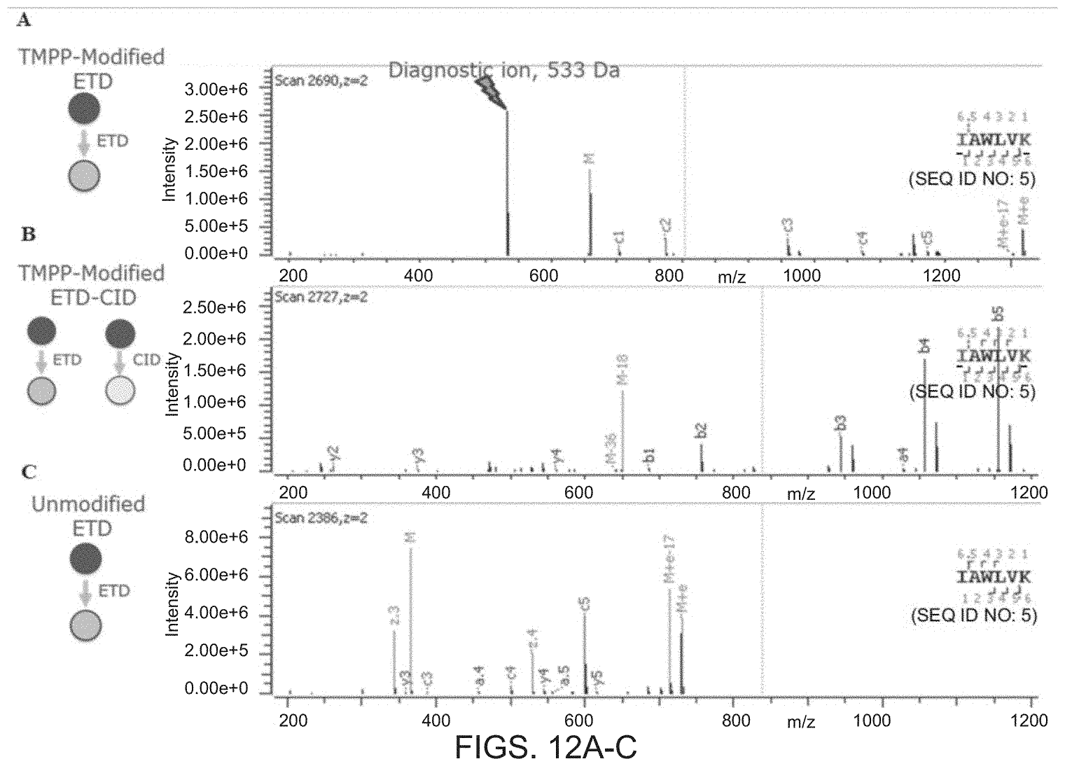

Previous studies have reported cathepsin D induced cleavage of GLP1 at W25/L26 (Dorai et al., Biotechnology Progress, 2011, 27:220-231; Deacon et al., Diabetes, 2004, 53:2181-2189; Manandhar et al., Journal of Medicinal Chemistry, 2015, 58:1020-1037). FIGS. 12A-C demonstrate that the MS² spectra showed evidence for neo-N termini generated due to protease activity. The product ion spectrum (FIG. 12A) resulting from ETD-MS² of the doubly charged ion produced a characteristic diagnostic ion TMPP⁺ (m/z=533 Da). In addition, c-type ions were predominantly observed in the TMPP site-localization. Site localization of TMPP on the N-termini was indicative of a neo N-termini due to F-I clip while a second TMPP on the C-terminal lysine residue can be inferred by a K-G clip since a TMPP conjugated to a lysine residue was resistant to trypsinization of the sample post-conjugation.

It was important to note that TMPP labeling also occurred on lysine and tyrosine residues and when peptides carrying TMPP modifications were subjected to ETD also generates diagnostic reporter ions and hence triggers CID-MS² events. These CID spectra were false positive identifications of reporters. Nevertheless, careful examination of the sequence ions in ETD and diagnostic ion triggered CID spectra site-specifically localized TMPP on the sequence and helped in the elimination of false-positives. FIG. 12B shows the diagnostic reporter ion (m/z=533) triggered CID-MS² spectrum of the doubly charged ions. The b- and y-type ions assisted in TMPP site-localization of both the N-terminal and C-terminal lysine. It was important to note that the peptide sequence did not generate characteristic CID induced reporter ions (m/z=573).

The ETD spectrum of the unconjugated peptide (FIG. 12C), where diagnostic ions were absent, was also generated. The product ion distribution of the unconjugated peptide gave a mixture of both c-type and z-type ions.

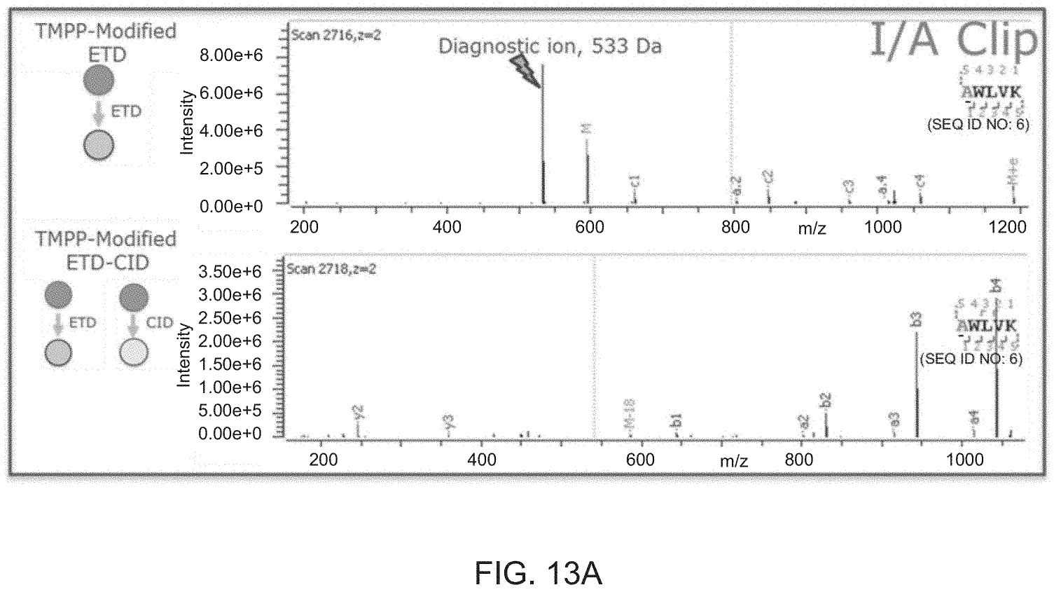

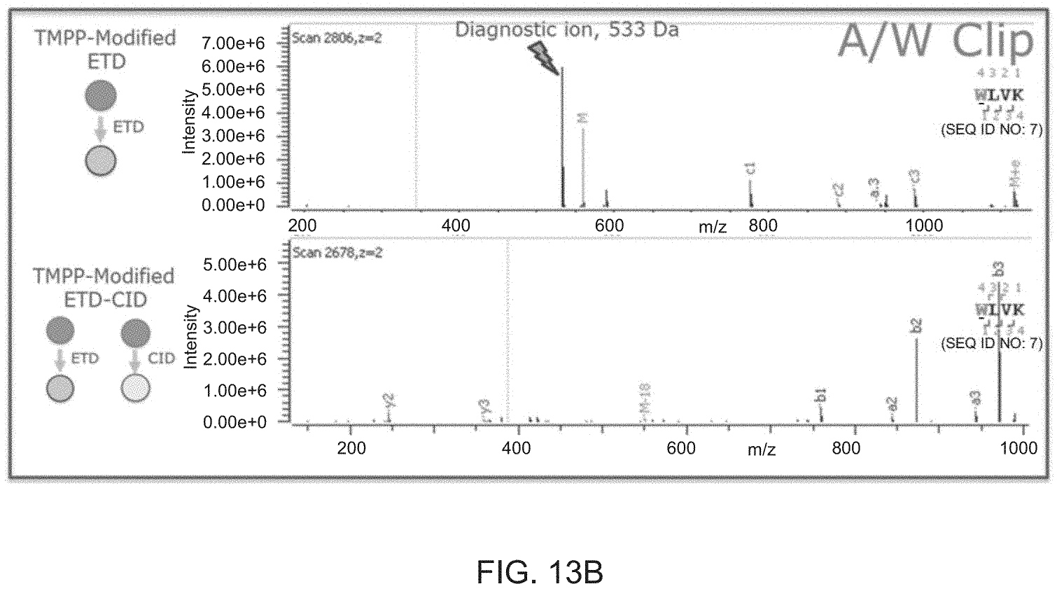

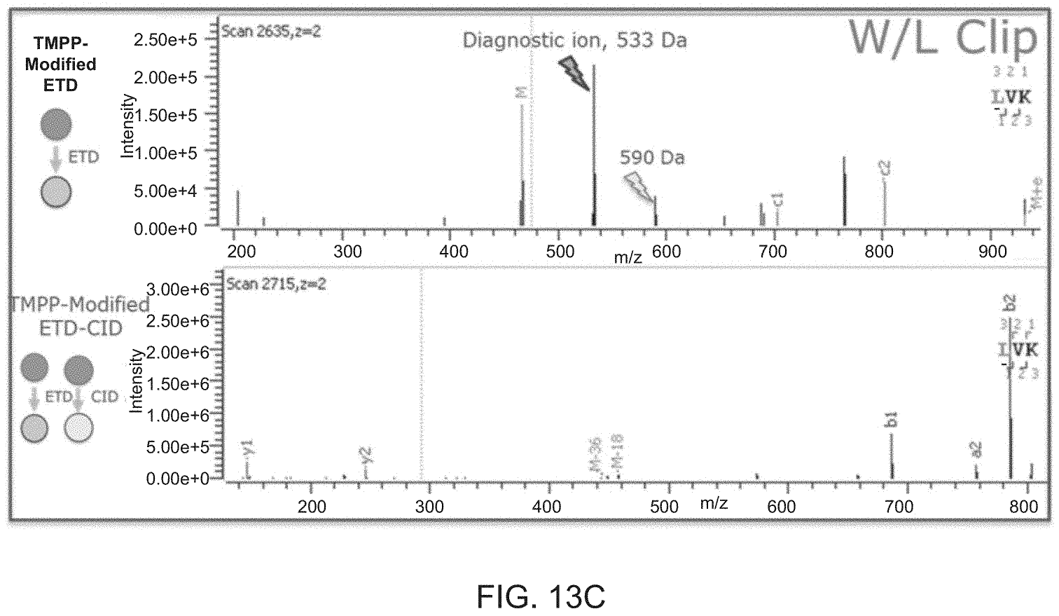

The complete analysis of dulaglutide peptides, resulted in identifying additional clipping of GLP1. FIGS. 13A-C show evidence of ETD-MS² and diagnostic ion triggered CID-MS² product ion spectra of surrogate peptides corresponding to the sequential clipping of GLP1 sequence. The surrogate peptides resulting from I/A clip generated exclusively a 533 Da diagnostic ion while those resulting from a A/W and W/L clip produced diagnostic ions at 533 Da and 590 Da during ETD. The predominant diagnostic ions at 533 Da triggered a CID-MS² event for each peptide, which generated a CID product ion spectrum. The CID-MS² spectra complemented the ETD identifications and the triggered MS² scans confirmed the presence of reporter ions generated from ETD-MS/MS in a seamless fashion to unambiguously identify neo N-termini for the entire data set.

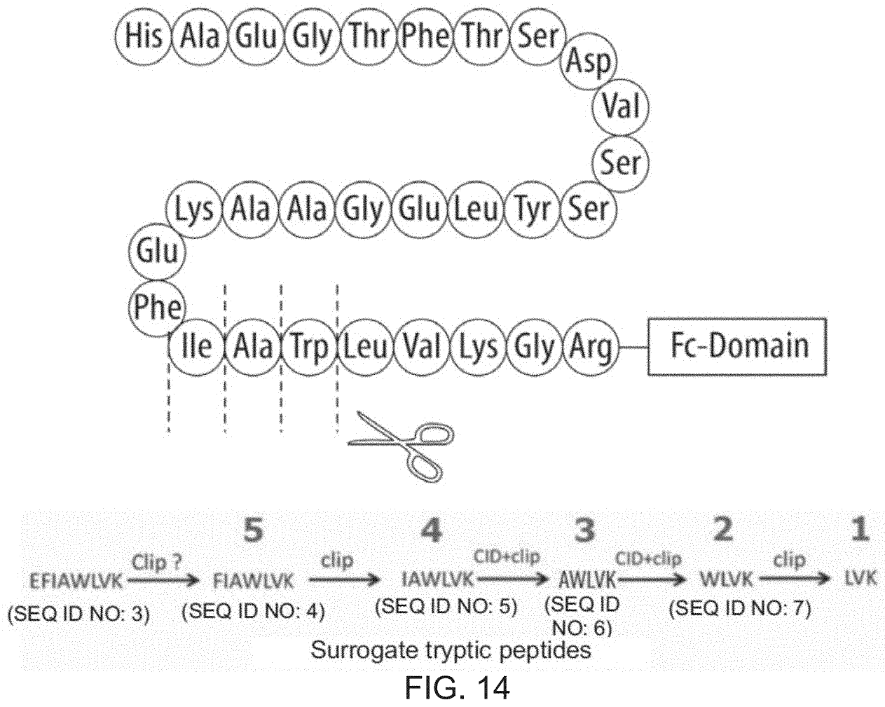

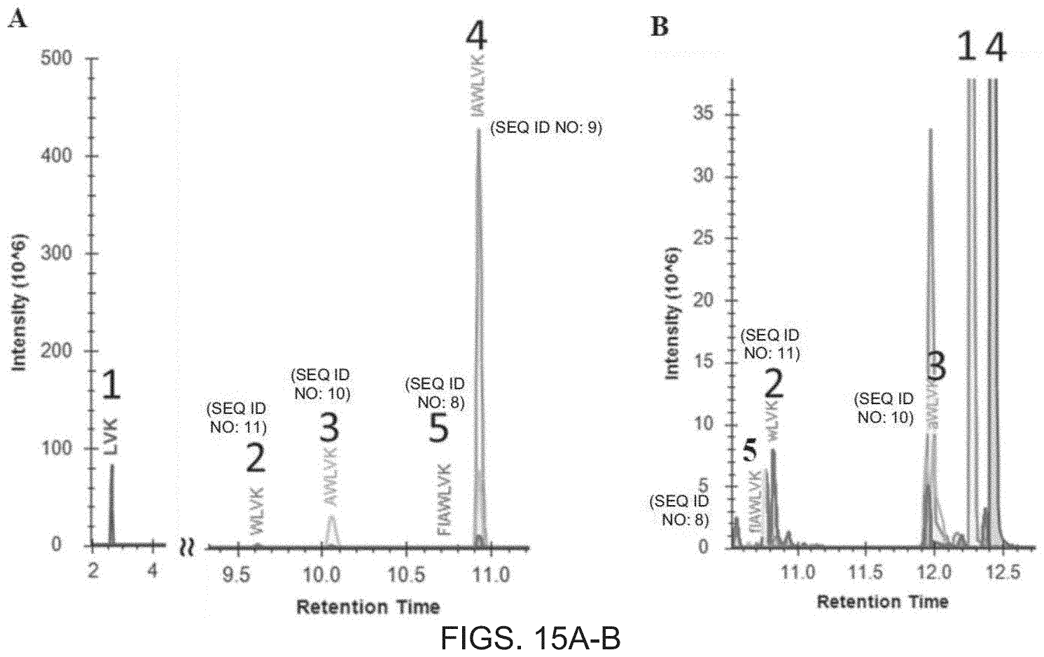

FIG. 14 shows clipping sites of dulaglutide to generate surrogate peptides from the neo N-termini. FIG. 15A shows the extracted ion chromatograms (XIC) of the surrogate peptides without TMPP labelling, and FIG. 15B shows chromatograms of the labeled surrogate peptides.

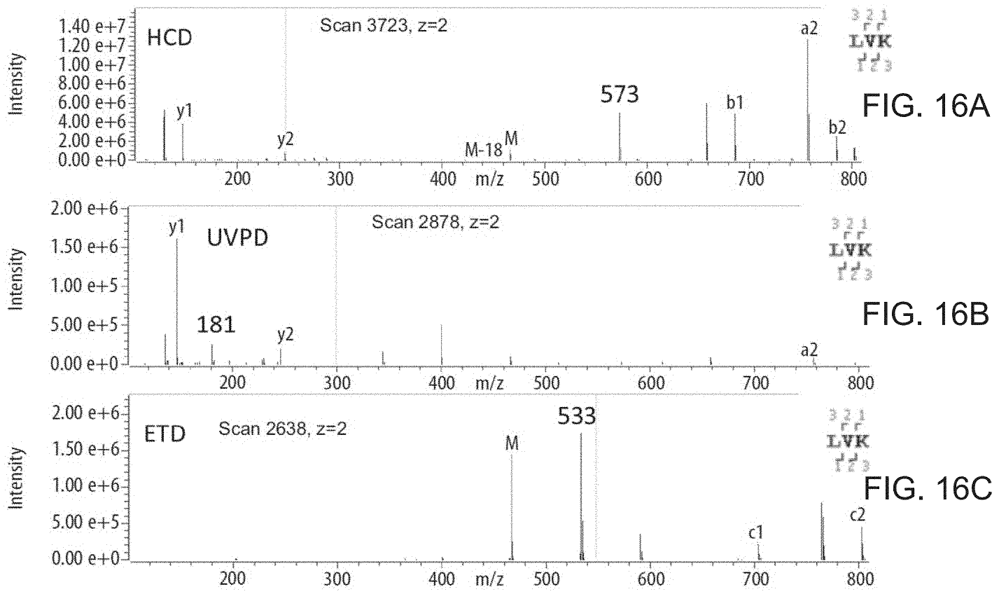

The utility of other dissociation modes, such as HCD, known to create more internal fragments (Michalski et al. Journal of Proteome Research, 2012, 11:5479-5491), and UVPD in addition to CID for generating reporter ions for TMPP labeled neo N-terminal peptides formed by clipping was further examined. Table 2 summarizes the results obtained for the series of clipped sites for surrogate peptides IAWLVK (SEQ ID NO: 5), AWLVK (SEQ ID NO: 6), WLVK (SEQ ID NO: 7) and LVK of the GLP1 sequence. While all these peptides generated the characteristic 533 Da diagnostic ions via ETD, only the LVK peptide sequences showed diagnostic ions for HCD and UVPD dissociation modes. As shown in FIGS. 16A and 16B, HCD produced a characteristic diagnostic ion at 573 Da due to amide bond dissociation (Sadagopan et al., Journal of the American Society for Mass Spectrometry, 2000, 11:107-119; He et al., Journal of the American Society for Mass Spectrometry, 2012, 23:1182-1190), and UVPD produced a diagnostic ion at 181 Da presumably due to further dissociation and rearrangement (Huang et al., Analytical Chemistry, 1997, 69:137-144). In addition to no detecting diagnostic ions in every peptide, significantly lower relative peak intensities were observed in contrast to the ETD generated diagnostic ions (FIG. 16C), which made triggering of these ions less informative.

TABLE 2
Dissociation Methods and Identified Clip sites of GLP1 based on reporter ions
TMPP-specific sequence ions (HCD, CID, and UVPD
diagnostic ion at 573 Da, 181 Da; ETD diagnostic ion at 533 Da).

| | Clipping Site by Diagnostic Ions | | | | Clipping Site by Sequence Ions | | | |
|---|---|---|---|---|---|---|---|---|
| | | | WLVK | | | | WLVK | |
| | IAWLVK | AWLVK | (SEQ | | IAWLVK | AWLVK | (SEQ | |
| Dissociation | (SEQ ID | (SEQ ID | ID NO: | | (SEQ ID | (SEQ ID | ID NO: | |
| Mode | NO: 5) | NO: 6) | 7) | LVK | NO: 5) | NO: 6) | 7) | LVK |
| HCD | No | No | No | Yes | Yes | Yes | Yes | Yes |
| CID | No | No | No | No | Yes | Yes | Yes | Yes |
| UVPD | No | No | No | Yes | Yes | No | No | Yes |
| ETD | Yes | Yes | Yes | Yes | Yes | Yes | Yes | Yes |
| Triggered | Yes | Yes | Yes | Yes | Yes | Yes | Yes | Yes |
| ETDCID | | | | | | | | |

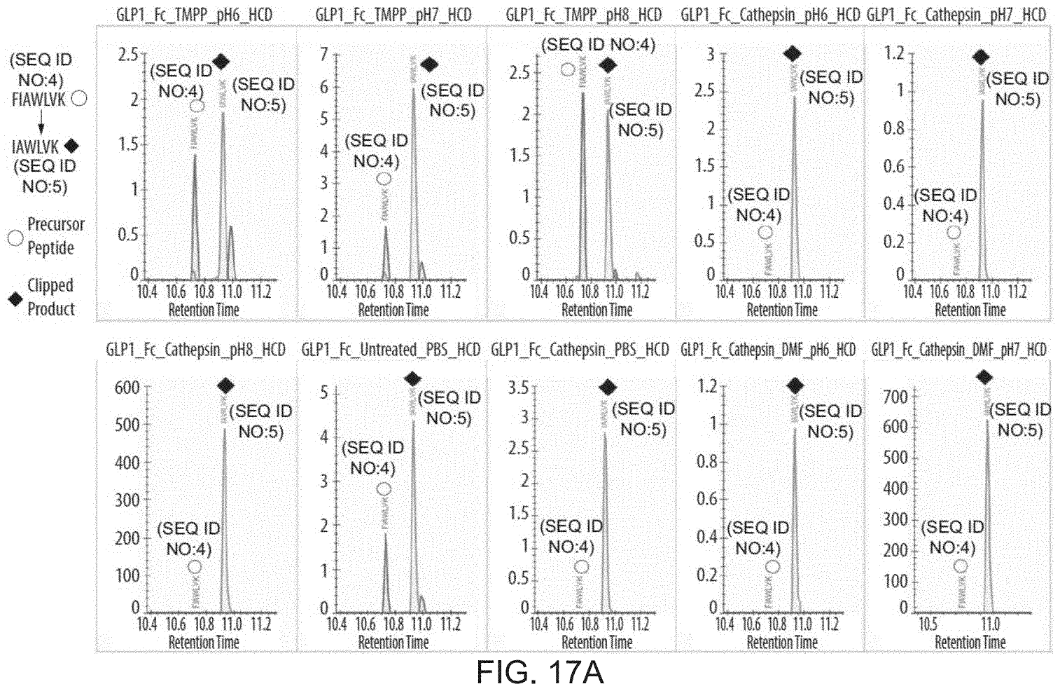

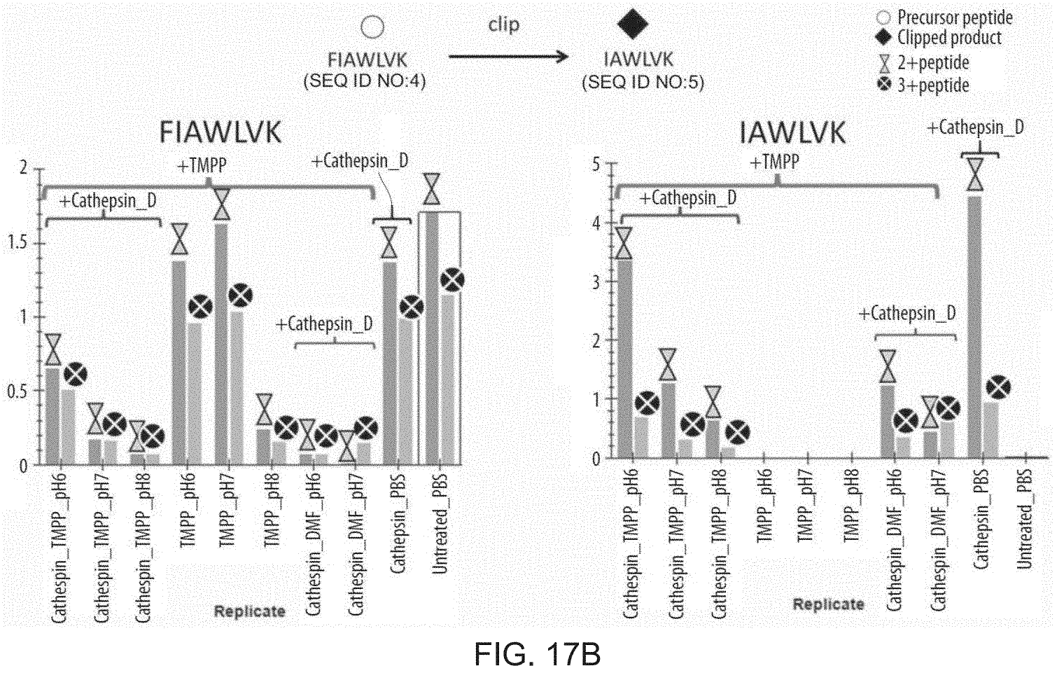

The XIC demonstrated that each TMPP labeled peptides eluted during the two rapid ramps between 10-13 min. The short LVK peptide predominantly observed in endogenous samples was less retentive and presumably elusive in peptide mapping experiments. However, the same peptide post TMPP labeling resulted in a significant column retention during reversed phase chromatography due to the overall hydrophobicity enhancement of the peptide. The fact that unlabeled peptides were also observed suggested that the TMPP labeling was not complete. The degree to which these TMPP derivatization reactions can be accomplished was investigated and optimum conditions were used for TMPP labeling (FIGS. 17A-17B). Despite the presence of the unlabeled counterpart that had different retention times from its precursor, it further validated the identities of the TMPP labeling of neo-N termini that resulted from clipping.

In conclusion, the use of TMPP labeling in conjunction with electron transfer dissociation mass spectrometry as a means of generating a facile diagnostic ion TMPP⁺ and TMPP-Ac-NH₂⁺ was reported. Among the reporter ions, ETD generated a facile TMPP⁺ reporter ion that was most intense for small tryptic peptides. This observation was unusual to the typical backbone dissociation efficiencies that usually increased with precursor ion charge for peptides with similar lengths. ETD efficiency of doubly charged ion was lower than triply or quadruply charged ions due to the creation of neutral product ions as a result of a single cleavage (Xia et al., Journal of the American Chemical Society, 2007, 129:12232-12243; Gunawardena et al., Journal of the American Chemical Society, 2005, 127:12627-12639). Thus, the data indicated that the fixed charge group on the TMPP moiety facilitated efficient electron recombination to produce a favorable fragment that retained the charge (Gunawardena et al., Molecular & Cellular Proteomics, 2016, 15:740-751). It was demonstrated that the various factors that affected the production of TMPP⁺ reporter ions using synthetic standard peptides of NIST monoclonal antibody as well by generating a large pool of peptides from K562 cell lysate with various lengths, charge states, and sequence compositions. TMPP⁺ reporter ion efficiency was highest for small doubly charged peptides. In contrast, HCD generated TMPP-Ac⁺ reporter ions that showed no charge state dependence. The diagnostic utility of ETD generated TMPP⁺ ions was determined by the AUC of 98% compared to AUC of 85% for HCD generated TMPP-AC+ ions of a ROC analysis. The ability to generate TMPP⁺ ions for triggered scans presented the complete interrogation of the sequence for accurate localization of the TMPP moiety or confirmation of the sequence with high confidence when ETD failed to generate enough backbone fragments of doubly charged ions. The high fidelity of triggered MS² was demonstrated for a panel of TMPP derivatized NIST synthetic peptides and tryptic peptides generated from GLP1-Fc fusion protein derivatized with TMPP. The labeling of the N-termini established the clipped site prior to digestion and mass spectrometry analysis, both of which were known to produce spurious fragments that can be mistaken for clipped sites.

Finally, the utility of TMPP⁺ diagnostic reporter ion-triggered MS² to examine Cathepsin-induced clipping-sites of the GLP1 was demonstrated. Evidence of ETD-MS² and diagnostic ion triggered MS2-CID product ion spectra of surrogate peptides corresponding to the sequential clipping of GLP1 sequence was obtained. The sequential clips were each confirmed with high confidence via TMPP⁺ diagnostic ions and subsequent reporter ion-triggered CID-MS². The CID-MS² spectra complimented the ETD identifications and triggered MS² scans provided a real time in silco filtering mechanism where a CID scan was only performed when the reporter ion was observed. The reporter ion triggering made for high confidence identification and seamless assembly of down neo N-termini for the entire data set. This mode of analysis reduced the ambiguity of clipped site detection where labeling was not performed and obviated the need to evaluate spurious artifacts created by sample digestion and mass spectrometry conditions.

In summary, protein therapeutics were susceptible to clipping via enzymatic and non-enzymatic mechanisms to render a neo N-termini of a degraded protein. The determination of neo N-termini of the therapeutic was typically performed via chemical derivatization of the N-terminal amine group by TMPP followed by proteolysis and mass spectrometric analysis.

The identification of TMPP labeled peptides were possible by mapping the peptide sequence with TMPP modification to the product ion spectrum derived from collisional activation. The site-specific localization of the TMPP tag allowed for unambiguous determination of the mature N-termini or neo N-termini. In addition to backbone product ions, TMPP reporter ions at 273 Da, formed via CID were diagnostic for the presence of a processed N-termini. However, reporter ions generated through CID were less informative due to their lower abundance. Herein it was demonstrated that a novel high-throughput LC-MS method for the facile generation of TMPP reporter ion at m/z 533 Da and in some instances 590 Da upon ETD. The abundant generation of these reporters allowed for subsequent MS/MS event using complementary ion activation modes, such as CID, HCD or UVPD, via intensity and m/z dependent triggering events to further sequence peptides.

More specifically, the utility of TMPP derived reporter-ions to identify clipped peptides via ETD-MS2 and diagnostic ion triggered MS2 events to autonomously filter clipped peptides was demonstrated. It was demonstrated that the herein described approach for efficient generation of reporter ions of TMPP labeled standard peptides that represented both N-terminal clipped species and undesirable TMPP labeling at lysine and tyrosine residues. The diagnostic utility of reporter ions, generated via ETD-MS2 over HCD-MS2 using large pool of TMPP labeled peptides with varying sequence compositions, lengths, and charge states, was also demonstrated. Finally, the herein described approach was applied to examine the sequential clipping of the GLP1 peptide for the commercially available GLP1 agonist Dulaglutide treated with Cathepsin D. Comparing the utility of TMPP reporter ions for complimentary dissociation modes: ETD, HCD, CID, and UVPD suggested that the facile charge loss peak at m/z=533 Da of TMPP⁺ ion generated via ETD was the most diagnostic for TMPP labeled peptide having neo N-termini. The rapid separation method allowed for TMPP labeled peptides to be separated efficiently from unlabeled peptides in complex samples and enhanced the retention time predictability of the TMPP labeled peptides to further improve the specificity and reduced false-positive identification of clipped peptides.

The materials and methods used in the above-mentioned experimental examples are now described.

### Chemicals and Reagents

(N-Succinimidyloxycarbonyl)tris(2,4,6-trimethoxyphenyl)phosphonium bromide (TMPP), 4-Morpholineethanesulfonic acid monohydrate (MES), N-(2-Hydroxyethyl)piperazine-N′-(2-ethanesulfonic acid) (HEPES), Trimethyl ammonium bicarbonate (TMAB), Sodium phosphate dibasic (Na2HPO4), sodium phosphate monobasic (NaH2PO4), Dimethylformamide (DMF), Cathepsin D from bovine spleen, 1,4 dithiothreitol (DTT), Iodoacetamide (IAA) and NIST-IgG1-KI monoclonal antibody were all purchased from Sigma (St Louis, Mo.).

Peptide standards from NIST monoclonal antibody was synthesized to 99.99% purity by Biomatik Corporation (Ontario, Canada). Human K562 predigest cell extract, and sequencing grade Trypsin and endoproteinase Lys-C were purchased from Promega (Madison, Wis.). GLP1-Fc fusion protein was purchased from Myoderm (Norristown, Pa.). Optima LC-MS grade acetonitrile, water, as well as formic acid, hydroxyl amine, Gybco PBS buffer solution were all purchased from Thermofisher Scientific (Waltham, Mass.).

### N-Terminal Labeling and Protein Digestion

Synthetic peptides, peptides from K562 predigest, NIST monoclonal antibody and GLP1-Fc fusion protein were all derivatized with TMPP. Derivatization was performed in the following buffers: 100 mM each of MES pH 6, HEPES pH 7, and sodium phosphate pH 8. A fresh 100 mM TMPP solution was prepared by dissolving 100 mg in 1.3 mL of DMF. TMPP labeling was performed by adapting a derivatization protocol published elsewhere (Deng et al., Methods in Molecular Biology, 2015, 1295:249-258).

In brief, 10 μl TMPP solution was added to 50 ug of peptides and proteins and mixed briefly followed by addition of 40 uL of the buffer, and then the resulting mixture was incubated for 1 hour. The reaction was quenched with 1 uL of hydroxyl amine and then lyophilized to dryness. The dried peptides were reconstituted with 0.1 FA water for MS and the dried proteins were reconstituted with TMAB for trypsinization.

Proteins were digested using a protocol described elsewhere (Gunawardena et al., Molecular & Cellular Proteomics, 2016, 15:740-751). In brief, proteins were reduced with DTT and subsequently alkylated with iodoacetamide. The proteins were then subjected to proteolysis with endoproteinase Lys-C for 1 h at 37° C., followed by 4-fold dilution 25 mM TMAB, pH 8.0, 1 mM CaCl₂) and further digested with trypsin for 4 h at 37° C. Digestion was stopped by the addition of formic acid to 0.10%. The peptide solutions were desalted on Sep-Pak Light C18 cartridges (Waters, Milford, Mass.) and collected for mass spectrometry.

### Instrumentation

All analysis was performed using an Agilent 1200 HPLC (Agilent Technologies, Santa Clara, Calif.) coupled to an Orbitrap Lumos (Thermo Scientific, San Jose, Calif.) Tribrid mass spectrometer equipped with an electrospray ion source using tune application software 2.1.1565.18 and Xcalibur 4.0.27.13.

### LC-MS/MS Analysis

All samples subjected to LC-MS/MS analysis were separated on an Agilent Infinity 1290 UHPLC (Agilent Technologies, Santa Clara, Calif.) using an AdvanceBio Peptide Map Micro Bore Rapid Resolution Column (1×150 mm, 2.7 μm) column at 65° C. The following 20 min rapid LC gradient program utilizing water with 0.1% formic acid as mobile phase A and acetonitrile as mobile phase B was employed: 0 min, 2% B; 10 min, 30% B; 10.5 min, 2% B; 11.5 min, 85% B; 12 min, 2% B; 13 min, 85% B; 13.5 min, 2% B; followed by wash step from 14-18 min, 85% B; and a subsequent re-equilibration for 2 min at 2% B. The 120 min LC gradient method utilized water with 0.1% formic acid as mobile phase A and acetonitrile as mobile phase B was employed: 0 min, 2% B; 60 min, 30% B; 60.5 min, 2% B; 61.5 min, 85% B; 62 min, 2% B; 63 min, 85% B; 63.5 min, 2% B; followed by wash step from 64.5-80 min, 85% B; and a subsequent re-equilibration for 20 min at 2% B. The flow rate in all gradients was set to 0.2 mL/min and the injection volume chosen was 2 μL. The mass spectrometer was operated in positive ionization mode with a data dependent MS² ETD, CID, HCD, UVPD methods. The interface conditions were as follows: emitter voltage, −2600 V; vaporizer temperature, 325° C.; ion transfer tube, 325° C.; sheath gas, 55 (arb); aux gas, 10 (arb); and sweep gas, 1 (arb).

### Method Settings

Internal mass spectrometer settings utilized for MS scans unless stated otherwise were as follows: RF lens, 60%; AGC (auto gain control) target, 4e5; maximum injection time, 50 ms; and 1μ scan in profile mode at 50K resolution on the Orbitrap mass analyzer. The method then sequentially included a series of filters prior to any HCD MS² events. A monoisotopic peak selection filter was included and set as peptide for all methods. An intensity filter of 1e5 was utilized for all methods unless stated otherwise. An optional charge state filter was included for some methods to select precursor charge states 2-6. An optional dynamic exclusion (DE) filter was included for some methods with either a 12 s or 3 s exclusion window and had common parameters of: exclude n=1 times; +/−3 ppm; exclude isotopes; and single charge state per precursor. Apex detection was included for one method and was set to: expected peak width, 6 s; desired apex window, 30%. There were five ddMS² OT-ETD scans with the following settings unless stated otherwise: quadrupole isolation, 2 m z isolation window; Reaction time of 50 ms detector type, Orbitrap, auto m z normal scan range, 15K resolution, 100 m/z first mass; AGC Target, 2e5, inject ions for all available parallelizable time, 50 ms maximum injection time; 1 μscan, profile. A targeted mass trigger (TMT) followed ddMS² IT-CID and included ions 533.193, 690.214; +/−5 ppm error tolerance; with the detection of either 2 or 1 ions from the list as explicitly stated; only ions within the top 10 most intense for all mass triggers. Subsequent ddMS² OT-CID conditions were as follows unless stated otherwise: MSⁿ Level, 2; quadrupole isolation, 1.6 m z isolation window; CID collision energy, 30; activation Q, 0.25; detector type, Orbitrap, auto m z normal scan range, 15K resolution; AGC Target, 5e4, inject ions for all available parallelizable time, 22 ms maximum injection time; 1 μscan, profile. The number of dependent scans between ddMS² OT-ETD and ddMS² IT-CID was set to 1. There were five ddMS² OT-HCD scans with the following settings unless stated otherwise: quadrupole isolation, 1.6 m/z isolation window; HCD collision energy, 40%, stepped 5%; detector type, Orbitrap, auto m/z normal scan range, 15K resolution, 100 m/z first mass; AGC Target, 5e4, inject ions for all available parallelizable time, 35 ms maximum injection time; 1 μscan, profile.

### Data Analysis

Data analysis was performed with Xcalibur visualization software from Thermo Scientific (San Jose, Calif.), Byos 3.9 chromatography and mass spectrometry data analysis software from Protein Metrics (Cupertino, Calif.), and R 3.6 statistical programming software (Vienna, Austria).

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of characterizing a N-tris(2,4,6-trimethoxyphenyl)phosphonium acetyl (TMPP) labeled peptide in a sample, comprising:
(i) subjecting the sample to an electron-induced dissociation tandem mass spectrometry to obtain a first mass spectrum of the TMPP labeled peptide;
(ii) identifying the TMPP labeled peptide by detecting or separating a TMPP reporter ion in the first mass spectrum of the TMPP labeled peptide;
(iii) subjecting the identified TMPP labeled peptide to a second mass spectrometry to thereby generate a second mass spectrum of the TMPP labeled peptide; and
(iv) characterizing the TMPP labeled peptide by analyzing the first mass spectrum and the second mass spectrum.

2. A method of characterizing a polypeptide, comprising:
(i) labeling the polypeptide with at least one reporter ion of claim 48 to obtain a TMPP labeled polypeptide;
(ii) digesting the TMPP labeled polypeptide to generate a mixture comprising one or more unlabeled peptides and one or more TMPP labeled peptides;
(iii) subjecting the mixture to liquid chromatography (LC) to generate elutes of the LC;
(iv) subjecting the elutes to an electron-induced dissociation tandem mass spectrometry to obtain a first mass spectrum of each of the one or more TMPP labeled peptides;
(v) identifying the one or more TMPP labeled peptides by detecting or separating a TMPP reporter ion in the first mass spectrum of each of the one or more TMPP labeled peptides;
(vi) subjecting the identified one or more TMPP labeled peptides to a second mass spectrometry to thereby generate a second mass spectrum of the each of the one or more TMPP labeled peptides; and
(vii) characterizing the polypeptide by analyzing the first mass spectrum and the second mass spectrum for each of the one or more TMPP labeled peptides.

3. A method of identifying a clipping site on a protein, comprising:
(i) obtaining a sample containing one or more clipped polypeptides of the protein;
(ii) labeling the one or more clipped polypeptides with at least one reporter ion of claim 48 to thereby obtain one or more TMPP labeled clipped polypeptides;
(iii) digesting the one or more TMPP labeled clipped polypeptides to generate a mixture comprising unlabeled peptides and TMPP labeled peptides;
(iv) subjecting the mixture to liquid chromatography (LC) to generate elutes of the LC;
(v) subjecting the elutes to an electron-induced dissociation tandem mass spectrometry to obtain a first mass spectrum of each of the TMPP labeled peptides;
(vi) identifying each of the TMPP labeled peptides by detecting or separating a TMPP reporter ion in the first mass spectrum for each of the TMPP labeled peptides;
(vii) subjecting each of the identified TMPP labeled peptides to a second mass spectrometry to thereby generate a second mass spectrum for each of the TMPP labeled peptides; and
(viii) identifying the clipping site on the protein by analyzing the first mass spectrum and the second mass spectrum for each of the TMPP labeled peptides.

4. The method of claim 1, wherein the electron-induced dissociation is electron transfer dissociation (ETD) or electron capture dissociation (ECD).

5.-6. (canceled)

7. The method of claim 1, wherein the TMPP reporter ion triggers the second mass spectrometry.

8. The method of claim 1, wherein the second mass spectrometry comprises collision-induced dissociation (CID), higher-energy collisional dissociation (HCD), or ultraviolet photodissociation (UVPD).

9-17. (canceled)

18. A method of identifying a clipping site on a protein, comprising:
(i) obtaining a sample containing one or more clipped polypeptides of the protein;
(ii) labeling the one or more clipped polypeptides with at least one reporter ion of claim 48 to thereby obtain one or more TMPP labeled clipped polypeptides;
(iii) digesting the one or more TMPP labeled clipped polypeptides to generate a mixture comprising unlabeled peptides and TMPP labeled peptides;
(iv) subjecting the mixture to liquid chromatography (LC) to generate elutes of the LC;
(v) subjecting the elutes to tandem mass spectrometry to thereby generate a first electron transfer dissociation (ETD) mass spectrum for each of the TMPP labeled peptides;
(vi) detecting or separating a TMPP reporter ion in the ETD mass spectrum for each of the TMPP labeled peptides;
(vii) upon detection or separation of the TMPP reporter ion, subjecting each of the TMPP labeled peptides to a second mass spectrometry, comprising a collision-induced dissociation (CID), higher-energy collisional dissociation (HCD), or ultraviolet photodissociation (UVPD), to thereby generate a CID, HCD, or UVPD mass spectrum for each of the TMPP labeled peptides, respectively; and
(viii) identifying the clipping site on the protein by analyzing the ETD mass spectrum and the CID, HCD, or UVPD mass spectrum for each of the TMPP labeled peptides.

19.-26. (canceled)

27. A system for identifying a clipping site on a polypeptide or characterizing a polypeptide in a sample, the system comprising a liquid chromatography (LC) device and a tandem mass spectrometer, wherein the tandem mass spectrometer comprises:
(i) a first ionization device;
(ii) a first mass to charge ratio filter or mass to charge ratio mass analyzer arranged and adapted in a first mode of operation to transmit ions having a mass to charge ratio within a first range;
(iii) a first ion mobility spectrometer, detector, or separator;
(iv) attenuation means for attenuating ions in a mode of operation;
(v) a control device configured to control the operation of the attenuation means so that ions having mass to charge ratios within the first range but having one or more undesired first charge states are substantially attenuated;
(vi) a second ionization device;
(vii) a second ion mobility spectrometer, detector, or separator; and
(viii) a data system configured to acquire non-mixed signals of fragment ions and to non-redundantly encode triggering ions, the non-redundant encoding being arranged to avoid or minimize repetitive overlapping of any two ion signals from different parent species at multiple repetitions of any individual gate time.

28. The system of claim 27, wherein the first ionization device is an electron-induced dissociation device.

29. (canceled)

30. The system of claim 27, wherein the second ionization device is a collision-induced dissociation (CID) device, higher-energy collisional dissociation (HCD) device, or ultraviolet photodissociation (UVPD) device.

31. The system of claim 27, wherein the mass spectrometer further comprises a collision device, fragmentation device, or reaction device.

32. The system of claim 27, wherein
a) the attenuation means comprises an ion gate or ion barrier;
b) the attenuation means is arranged downstream of the ion mobility spectrometer or separator;
or a combination thereof.

33. (canceled)

34. The system of claim 27, wherein
a) the first mass to charge ratio filter or mass to charge ratio mass analyzer is arranged and adapted in the first mode of operation to attenuate ions having mass to charge ratios outside of the first range;
b) the first mass to charge ratio filter or mass to charge ratio mass analyzer is arranged upstream or downstream of said ion mobility spectrometer or separator;
or a combination thereof.

35-36. (canceled)

37. The system of claim 27 further comprising an ion guide, ion trap or ion trapping region arranged upstream of said ion mobility spectrometer or separator,
wherein said ion guide, ion trap or ion trapping region is arranged to trap, store or accumulate ions and then to periodically pulse ions into or towards said ion mobility spectrometer or separator.

38. The system of claim 27, wherein the sample is subject to the LC device to generate elutes.

39.-41. (canceled)

42. The system of claim 27, wherein the clipping site on the polypeptide or the polypeptide is labeled with N-tris(2,4,6-trimethoxyphenyl)phosphonium acetyl (TMPP).

43.-47. (canceled)

48. A reporter ion for identifying a clipping site on a polypeptide or characterizing a polypeptide,
wherein the clipping site or the polypeptide is labeled with N-tris(2,4,6-trimethoxyphenyl)phosphonium acetyl (TMPP), and
wherein the TMPP is ionized to generate the reporter ion.

49. (canceled)

50. The reporter ion of claim 48, wherein the TMPP is ionized by a mass spectrometer to generate the reporter ion.

51.-54. (canceled)

55. A composition for identifying a clipping site on a polypeptide or characterizing a polypeptide, wherein the composition comprises at least one reporter ion of claim 48 and a polypeptide.

56. (canceled)

57. A kit for identifying a clipping site on a polypeptide or characterizing a polypeptide in a sample, the kit comprising:
(i) at least one reported ion claim 48 for labeling the clipping site on the polypeptide or for labeling the polypeptide; and
(ii) an instructional material.


SEQUENCE LISTING (31 sequences)

SEQ ID NO: 1 - protein, 11 aa, Artificial Sequence
  TMPP tagged NIST antibody
  MISC_FEATURE (1)..(1): TMPP labeled at Asp
DIQMTQSPSTL

SEQ ID NO: 2 - protein, 275 aa, Homo sapiens
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 8 aa, Homo sapiens
EFIAWLVK

SEQ ID NO: 4 - protein, 7 aa, Homo sapiens
FIAWLVK

SEQ ID NO: 5 - protein, 6 aa, Homo sapiens
IAWLVK

SEQ ID NO: 6 - protein, 5 aa, Homo sapiens
AWLVK

SEQ ID NO: 7 - protein, 4 aa, Homo sapiens
WLVK

SEQ ID NO: 8 - protein, 7 aa, Artificial Sequence
  TMPP tagged
  MISC_FEATURE (1)..(1): TMPP labeled at Phe
FIAWLVK

SEQ ID NO: 9 - protein, 6 aa, Artificial Sequence
  TMPP tagged
  MISC_FEATURE (1)..(1): TMPP labeled at Ile
IAWLVK

SEQ ID NO: 10 - protein, 5 aa, Artificial Sequence
  TMPP tagged
  MISC_FEATURE (1)..(1): TMPP labeled at Ala
AWLVK

SEQ ID NO: 11 - protein, 4 aa, Artificial Sequence
  TMPP tagged
  MISC_FEATURE (1)..(1): TMPP labelled at Trp
WLVK

SEQ ID NO: 12 - protein, 5 aa, Artificial Sequence
  Synthetic Peptide
  MISC_FEATURE (1)..(1): TMPP labeled at Ala
ADYEK

SEQ ID NO: 13 - protein, 17 aa, Artificial Sequence
  Synthetic Peptide
  MISC_FEATURE (1)..(1): TMPP labeled at Val
VYACEVTHQGLSSPVTK

SEQ ID NO: 14 - protein, 4 aa, Artificial Sequence
  Synthetic Peptide
  MISC_FEATURE (1)..(1): TMPP labeled at Ser
SFNR

SEQ ID NO: 15 - protein, 4 aa, Artificial Sequence
  Synthetic Peptide
  MISC_FEATURE (1)..(1): TMPP labeled at Val
VQWK

SEQ ID NO: 16 - protein, 7 aa, Artificial Sequence
  Synthetic Peptide
  MISC_FEATURE (1)..(1): TMPP labeled at Asp
DTLMISR

SEQ ID NO: 17 - protein, 14 aa, Artificial Sequence
  Synthetic Peptide
  MISC_FEATURE (1)..(1): TMPP labeled at Phe
FNWYVDGVEVHNAK

SEQ ID NO: 18 - protein, 4 aa, Artificial Sequence
  Synthetic Peptide
  MISC_FEATURE (1)..(1): TMPP labeled at Thr
TKPR

SEQ ID NO: 19 - protein, 9 aa, Artificial Sequence
  Synthetic Peptide
  MISC_FEATURE (1)..(1): TMPP labeled at Glu
EEQYNSTYR

SEQ ID NO: 20 - protein, 16 aa, Artificial Sequence
  Synthetic Peptide
  MISC_FEATURE (1)..(1): TMPP labeled at Val
VVSVLTVLHQDWLNGK

SEQ ID NO: 21 - protein, 4 aa, Artificial Sequence
  Synthetic Peptide
  MISC_FEATURE (1)..(1): TMPP labeled at Gly
GQPR

SEQ ID NO: 22 - protein, 11 aa, Artificial Sequence
  Synthetic Peptide
  MISC_FEATURE (1)..(1): TMPP labeled at Glu
EPQVYTLPPSR

SEQ ID NO: 23 - protein, 15 aa, Artificial Sequence
  Synthetic Peptide
  MISC_FEATURE (1)..(1): TMPP labeled at Ser
STSGGTAALGCLVKD

SEQ ID NO: 24 - protein, 26 aa, Artificial Sequence
  Synthetic Peptide
  MISC_FEATURE (1)..(1): TMPP labeled at Ser
STSGGTAALGCLVKDYFPEPVTVSWN

SEQ ID NO: 25 - protein, 16 aa, Artificial Sequence
  Synthetic Peptide
  MISC_FEATURE (1)..(1): TMPP labeled at Val
VVSLTVLHQDWLNGKE

SEQ ID NO: 26 - protein, 15 aa, Artificial Sequence
  Synthetic Peptide
  MISC_FEATURE (1)..(1): TMPP labeled at Val
VVSLTVLHQDWLNGK

SEQ ID NO: 27 - protein, 14 aa, Artificial Sequence
  Synthetic Peptide
  MISC_FEATURE (1)..(1): TMPP labeled at Val
VSLTVLHQDWLNGK

SEQ ID NO: 28 - protein, 10 aa, Artificial Sequence
  Synthetic Peptide
IIQLLDDYPK

SEQ ID NO: 29 - protein, 8 aa, Artificial Sequence
  Synthetic Peptide
GFGFVLFK

SEQ ID NO: 30 - protein, 10 aa, Artificial Sequence
  Synthetic Peptide
IMLPWDPTGK

SEQ ID NO: 31 - protein, 5 aa, Artificial Sequence
  Synthetic Peptide
SFNIR